(19) United States
(12) Patent Application Publication
(10) Pub. No.: US 2024/0093296 A1
(43) Pub. Date: Mar. 21, 2024

(54) ANTIGEN-BINDING PROTEIN, MUTANT PEPTIDE COMPLEMENTARITY SCORING AND USES THEREOF

(71) Applicant: University of South Florida, Tampa, FL (US)

(72) Inventors: George Blanck, Tampa, FL (US); Boris Il'ich Chobrutskiy, Tampa, FL (US); Michelle Yeagley, Tampa, FL (US); Andrea Diviney, Tampa, FL (US); Juan Arturo, Tampa, FL (US)

(21) Appl. No.: 18/502,509

(22) Filed: Nov. 6, 2023

Related U.S. Application Data
(63) Continuation of application No. 16/702,467, filed on Dec. 3, 2019.
(60) Provisional application No. 62/927,482, filed on Oct. 29, 2019.
(60) Provisional application No. 62/927,476, filed on Oct. 29, 2019.
(60) Provisional application No. 62/790,719, filed on Jan. 10, 2019.
(60) Provisional application No. 62/774,583, filed on Dec. 3, 2018.

(51) Int. Cl. C12Q 1/6876; C12Q 1/6886; G01N 33/68; G16B 20/00

(52) U.S. Cl. CPC C12Q 1/6876; C12Q 1/6886; G01N 33/6854; G16B 20/00; C12Q 2600/106; C12Q 2600/118; G01N 2500/00

(57) ABSTRACT

The present disclosure relates to methods of determining complementarity scores of antigen-binding proteins and proteins associated with cancers and uses thereof for diagnosing and treating cancers and for screening antigens and antigen-binding proteins.

### CROSS REFERENCE TO RELATED APPLICATIONS

This application claims the benefit of U.S. Provisional Patent Application Ser. No. 62/774,583 filed on Dec. 3, 2018; U.S. Provisional Patent Application Ser. No. 62/790,719 filed on Jan. 10, 2019; U.S. Provisional Patent Application Ser. No. 62/927,482 filed on Oct. 29, 2019; and U.S. Provisional Patent Application Ser. No. 62/927,476 filed on Oct. 29, 2019, the disclosure of which is expressly incorporated herein by reference.

### FIELD

The present disclosure relates to methods of determining complementarity scores of antigen-binding proteins and proteins associated with cancers and uses thereof for diagnosing and treating cancers and for screening antigens and antigen-binding proteins.

### BACKGROUND

T-lymphocytes, B cells, and antibodies are key components in the immunological defense against cancers. While it is known that mutant tumor peptides are at the heart of attracting T-infiltrating lymphocytes (TILs) and are thereby collectively stimulating T cell killing, effective approaches for improving adaptive immune cell-dependent immunotherapy are still inadequate. It is unknown whether large numbers of mutant peptides in tumors provide for a versatile and thereby effective adaptive immune responses. Therefore, what is needed are new methods for diagnosing and treating cancer patients. The methods disclosed herein address these and other needs.

### SUMMARY

Disclosed herein are methods for determining complementarity between antigen-binding proteins and proteins associated with cancers and uses thereof in treating, diagnosing, and/or prognosing cancers.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and a second polynucleotide encoding a protein associated with the cancer;
determining a complementarity score of the CDR domain and the protein associated with the cancer, comprising multiplying a net charge per residue (NCPR) of the CDR domain by a value of change in charge due to an amino acid substitution in the protein associated with the cancer and further by “−1”;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a complementarity score of the CDR domain and the protein associated with the cancer, comprising multiplying a Uversky hydropathy score of the CDR domain by a value of change in Uversky hydropathy score due to an amino acid substitution in the protein associated with the cancer;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer;
determining a mean z-score by averaging a first z-score of the first complementarity score and a second z-score of the second complementarity score, wherein the first z-score and the second z-score are relative to a reference database;
and
predicting one of:
   a. the subject as having a shorter overall survival if the mean z-score is lower in the biological sample derived from the subject compared to a reference control, or
   b. the subject as having a longer overall survival if the mean z-score is higher in the biological sample derived from the subject compared to a reference control.

In some embodiments, the antigen-binding protein comprises a T cell receptor (TCR) alpha chain or a TCR beta chain. In some embodiments, the antigen-binding protein comprises a B cell receptor (BCR) or an antibody. In some embodiments, the antigen-binding protein comprises a heavy chain of a B cell receptor or an antibody. In some embodiments, the CDR domain is a CDR3 domain.

In some embodiments, the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some embodiments, the protein associated with the cancer is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

In some embodiments, the method of any preceding aspect further comprises administering to the subject an anti-cancer agent. In some embodiments, the anti-cancer agent is selected from the group consisting of cordycepin, fenretinide, Zyclara, vemurafenib (Zelboraf®), dabrafenib (Tafinlar®), encorafenib (Braftovi®), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex®), irinotecan (Camptosar®), mitomycin, doxorubicin (Adriamycin), methotrexate, vinblastine (CMV), durvalumab (Imfinzi®), avelumab (Bavencio®), Erdafitinib (Balversa), dacarbazine (DTIC), epirubicin, temozolomide (Temodar®), gemcitabine (Gemzar®), trabectedin (Yondelis®), and Pazopanib (Votrient).

In some aspects, disclosed herein is a method for screening an antigen-binding protein for treating a cancer, comprising:
obtaining a first sequence of a CDR domain of the antigen-binding protein and a second sequence of a protein associated with the cancer;
determining a complementarity score of the CDR domain and the protein associated with the cancer; and
determining the antigen-binding protein for treating the cancer if the complementarity score thereof is higher compared to a reference control.

In some embodiments, the complementarity score of the CDR domain of the antigen-binding protein and the protein associated with the cancer is determined by multiplying a net charge per residue (NCPR) of the CDR domain by a value of change in charge due to an amino acid substitution in the protein associated with the cancer and further by “−1”.

In some embodiments, the complementarity score of the CDR domain of the antigen-binding protein and the protein associated with the cancer is determined by multiplying a Uversky hydropathy score of the CDR domain by a value of change in Uversky hydropathy score due to an amino acid substitution in the protein associated with the cancer.

In some embodiments, the complementarity score of the CDR domain of the antigen-binding protein and the protein associated with the cancer is determined by
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer; and
determining a mean z-score by averaging a first z-score of the first complementarity score and a second z-score of the second complementarity score, wherein the first z-score and the second z-score are relative to a reference database; and wherein the complementarity score is equal to the mean z-score.

### BRIEF DESCRIPTION OF THE DRAWINGS

The accompanying figures, which are incorporated in and constitute a part of this specification, illustrate several aspects described below.

FIGS. 1A and 1B show survival rates associated with TRA CDR3 isoelectric points. FIG. 1A shows Kaplan-Meyer (KM) analysis of OS represented by TCGA BLCA barcodes representing the highest 50% (grey) and lowest 50% (black) TRA CDR3 isoelectric points (left panel); and representing the highest 20% (grey) and lowest 20% (black) TRA CDR3 isoelectric points (right panel). Median OS for highest 50% isoelectric point group, 28.22 months. Median OS for lowest 50% isoelectric point group, 59.26 months. Log rank comparison p-value 0.0278. Median OS for highest 20% isoelectric point group, 20.47 months. Median OS for lowest 20% isoelectric point group, 97.04 months. Log rank comparison p-value 0.0013. FIG. 1B shows KM analysis of DFS for BLCA barcodes representing the highest 50% (grey) compared to lowest 50% (black) of TRA CDR3 isoelectric points (left panel); and representing the highest 20% (grey) compared to lowest 20% (black) of TRA CDR3 isoelectric points (right panel). Median DFS for highest 50% isoelectric point group, 24.64 months. Median DFS for lowest 50% isoelectric point group, 72.34 months. Log rank comparison p-value 0.0125. Median DFS for highest 20% isoelectric point group, 28.06 months. Median DFS for lowest 20% isoelectric point group, 82.42 months. Log rank comparison p-value 0.0930.

FIGS. 2A-2D show survival distinctions within mutated gene groups, based on TRA CDR3 isoelectric point values. FIG. 2A shows KM analysis of OS for BLCA barcodes representing the highest 50% (grey) compared to the lowest 50% (black) of TRA CDR3 isoelectric points, among barcodes representing PIK3CA gene mutations (left panel); and representing the highest 50% (grey) compared to lowest 50% (black) TRA CDR3 isoelectric points among barcodes that did not have any PIK3CA gene mutations (right panel). Median OS for the highest 50% isoelectric point group, in the PIK3CA mutated barcode set, 22.14 months. Median OS for lowest 50% isoelectric point group, in the PIK3CA mutated barcode set, 104.6 months. Log rank comparison p-value 0.0287. Median OS for the highest 50% isoelectric point group in the barcodes set lacking PIK3CA mutations, 28.22 months. Median OS for the lowest 50% isoelectric point group in the barcode set lacking PIK3CA mutations, 44.28 months. Log rank comparison p-value 0.0953. FIG. 2B shows KM analysis of DFS for BLCA barcodes representing the highest 50% (grey) compared to the lowest 50% (black) TRA CDR3 isoelectric points among barcodes representing PIK3CA gene mutations (left panel); and representing the highest 50% (grey) compared to the lowest 50% (black) TRA CDR3 isoelectric points among barcodes that did not have any PIK3CA gene mutations (right panel). Median DFS for the highest 50% isoelectric point group in the PIK3CA mutated barcode set, 20.37 months. Median DFS for lowest 50% isoelectric point group in the PIK3CA mutated barcode set, 101.4 months. Log rank comparison p-value 0.0036. Median DFS for highest 50% isoelectric point group in the barcode set lacking PIK3CA mutations, 28.06 months. Median DFS for lowest 50% isoelectric point in the barcode set lacking PIK3CA mutations, 44.15 months. Log rank comparison p-value 0.6423. FIG. 2C shows KM analysis of OS for BLCA barcodes representing the highest 50% (grey) compared to the lowest 50% (black) TRA CDR3 isoelectric points among the barcodes representing ITPR2 gene mutations (left panel); and representing the highest 50% (grey) compared to the lowest 50% (black) TRA CDR3 isoelectric points among barcodes that did not have any ITPR2 gene mutations (right panel). Median OS for highest 50% isoelectric point group in the ITPR2 mutated barcode set, 15.11 months. Median OS for lowest 50% isoelectric point group in the ITPR2 mutated barcode set, undefined. Log rank comparison p-value 0.0052. Median OS for highest 50% isoelectric point group in the barcode set lacking ITPR2 mutations, 31.18 months. Median OS for lowest 50% isoelectric point group in the barcode set lacking ITPR2 mutations, 44.91 months. Log rank comparison p-value 0.1961. FIG. 2D shows KM analysis of DFS for BLCA barcodes representing the highest 50% (grey) compared to lowest 50% (black) TRA CDR3 isoelectric points among the barcodes representing ITPR2 gene mutations (left panel); and representing the highest 50% (grey) compared to the lowest 50% (black) TRA CDR3 isoelectric points among barcodes that did not have any ITPR2 gene mutations (right panel). Median DFS for highest 50% isoelectric point group in the ITPR2 mutated barcode set, 20.37 months. Median DFS for lowest 50% isoelectric point in the ITPR2 mutated barcode set, undefined. Log rank comparison p-value 0.0006. Median DFS for highest 50% isoelectric point group in the barcode set lacking ITPR2 mutations, 24.9 months. Median DFS for lowest 50% isoelectric point group in the barcode set lacking ITPR2 mutations, 51.41 months. Log rank comparison p-value 0.1812.

FIGS. 3A-3C show additional TRA CDR3 physico-chemical parameters distinguishing BLCA survival rates. FIG. 3A shows KM analysis of OS for BLCA barcodes representing the highest 50% (grey) compared to the lowest 50% (black) TRA CDR3 net charge per residue (NCPR)(left panel); and representing the highest 20% (grey) compared to lowest 20% (black) TRA CDR3 NCPR (right panel). Median OS for the highest 50% NCPR group, 28.22 months. Median OS for the lowest 50% NCPR group, 59.26 months. Log rank comparison p-value, 0.0330. Median OS for the highest 20% NCPR group, 22.14 months. Median OS for the lowest 20% NCPR group, 46.65 months. Log rank comparison p-value, 0.0111. FIG. 3B shows KM analysis of OS for BLCA barcodes representing the highest 50% (grey) compared to the lowest 50% (black) for the TRA CDR3 fraction of positive residues (left panel); and representing the highest 20% (grey) compared to the lowest 20% (black) [right] TRA CDR3 fraction of positive residues. Median OS for highest 50% fraction of positive residues group, 29.7 months. Median OS for lowest 50% fraction of positive residues group, 66.36 months. Log rank comparison p-value, 0.0381. Median OS for highest 20% fraction of positive residues group, 16.69 months. Median OS for lowest 20% fraction of positive residues group, 44.28 months. Log rank comparison p-value, 0.0014. FIG. 3C shows KM overall survival curves for BLCA barcodes representing the highest 50% (grey) compared to the lowest 50% (black) of TRA CDR3 fraction of tiny residues (left panel); and representing the highest 20% (grey) compared to lowest 20% (black) of TRA CDR3 fraction of tiny residues (right panel). Median OS for highest 50% fraction of tiny residues group, 33.11 months. Median OS for lowest 50% fraction of tiny residues group, 41.21 months. Log rank comparison p-value, 0.4523. Median OS for highest 20% fraction of tiny residues group, 104.6 months. Median OS for lowest 20% fraction of tiny residues group, 17.71 months. Log rank comparison p-value, 0.0079.

FIGS. 4A-4C show TRA CDR3 physico-chemical properties distinguishing survival rates for additional cancer. FIG. 4A shows KM analysis of OS for ESCA barcodes representing the highest 50% (grey) compared to the lowest 50% (black) TRA CDR3 isoelectric points (left panel); and KM analysis of DFS for ESCA barcodes representing the highest 50% (grey) compared to the lowest 50% (black) of TRA CDR3 isoelectric points (right panel). Median OS for the highest 50% isoelectric point group, 14.29 months. Median OS for the lowest 50% isoelectric point group, 52.53 months. Log rank comparison p-value, 0.0410. Median DFS for the highest 50% isoelectric point group, 8.87 months. Median DFS for the lowest 50% isoelectric point group, 37.42 months. Log rank comparison p value, 0.0043. FIG. 4B shows KM analysis of OS for STAD barcodes representing the highest 50% (grey) compared to the lowest 50% (black) of TRA CDR3 fraction of positive residues. Median OS for highest 50% fraction of positive residues group, 25.03 months. Median OS for lowest 50% fraction of positive residues, 68.99 months group. Log rank comparison p-value, 0.0154. FIG. 4C shows KM analysis of OS for OVCA barcodes representing the highest 50% (grey) compared to the lowest 50% (black) TRA CDR3 fraction of positive residues. Median OS for the highest 50% fraction of positive residues group, 55.12 months. Median OS for lowest 50% fraction of positive residues group, 34.79 months. Log rank comparison p-value 0.0147.

FIGS. 5A-5C show stratification of STS Patients by TCR-α CDR3 and Tumor Antigen Complementarity. The indicated calculations of complementarity, or lack of complementarity, represent the recovered TCR-α CDR3s and the entire mutanome for each STS patient. Patient groups are indicated in the figure.

FIG. 6 shows pro-apoptosis Gene Expression Increased in STS Patients with TCR-α CDR3 and Tumor Antigen Complementarity. The average GZMB RNASeq values for tumors with a complementary CDR3-mutanome (left box), and with a non-complementary CDR3-mutanome (right box), were 264.75 and 124.11, respectively.

FIG. 7 shows pro-apoptotic gene RNA expression distinctions between tumors with and without TRA recombination read recoveries. The average CASP4 RNASeq values for tumors with TRA recoveries (left box) and without TRA recoveries (right box) were 1399.3 and 865.1, respectively, p<0.001. For CASP5, average RNASeq values were 13.13 and 5.92, p=0.0014. For GZMA, average RNASeq values were 573.68 and 174.06, p<0.001. For GZMB, average RNASeq values were 162.03 and 61.69, p<0.001.

FIGS. 8A and 8B show survival advantage associated with NCPR complementarity between TRA CDR3s and the corresponding tumor mutanome. FIG. 8A shows KM overall survival (OS) analysis for TCGA-SARC case IDs representing a complementary CDR3-mutanome (n=16, black), compared to case IDs representing a non-complementary CDR3-mutanome (n=37, grey). Median OS for the SARC case IDs representing a complementary CDR3-mutanome, undefined; for case IDs representing a non-complementary CDR3-mutanome, 84.59 months. Log rank p-value=0.0174. FIG. 8B shows the average GZMB RNASeq values for tumors with a complementary CDR3-mutanome (left box), and with a non-complementary CDR3-mutanome (right box), were 264.75 and 124.11, respectively, p=0.0347.

FIGS. 9A and 9B show distinct OS rates represented by mutant and wild-type TP53 when differentiated by tumor TRA recovery. FIG. 9A shows KM OS analysis for SARC case IDs represented by a mutant TP53, with tumor TRA recoveries (n=17, black) and without tumor TRA recoveries (n=59, grey). Median OS for SARC case IDs represented by a mutant TP53 with TRA recoveries, undefined; without TRA recoveries, 48.55 months. Log rank comparison p-value=0.0421. FIG. 9B shows KM OS analysis for SARC case IDs represented by wild-type TP53, with TRA recoveries (n=40, black) and without TRA recoveries (n=145, grey). Median OS for SARC case IDs with wild-type TP53 and with TRA recoveries, 84.59 months; without TRA recoveries, 65.41 months. Log rank comparison p-value=0.3014.

FIGS. 10A-10D show survival distinctions for case IDs representing CDR3-mutant TP53 complementarity using RNASeq derived TRA recombination reads. FIG. 10A shows KM OS analysis for SARC case IDs representing (RNASeq-based) TRA recombination read recoveries from their tumors (black, n=189), compared with no TRA recombination (grey, n=72). Median OS for SARC case IDs representing TRA recombination reads, 80.42 months; representing no TRA recombination reads, 46.78 months. Log rank comparison p-value=0.0389. FIG. 10B shows KM OS analysis for SARC case IDs representing tumors having a mutant TP53, with (n=56, black) and without (n=20, grey) TRA recombination reads. Median OS for SARC case IDs with TRA recombination reads, 85.38 months; representing no TRA recombination reads, 34.86 months. Log rank p-value=0.0065. FIG. 10C shows KM OS analysis for SARC case IDs representing tumors having wild-type TP53, with (n=133, black) and without (n=52, grey) TRA recombination reads. Median OS for SARC case IDs TRA recombination reads, 76.35 months; representing no TRA recombination reads, 63.76 months. Log rank p-value=0.4028. FIG. 10D shows KM OS analysis for SARC case IDs representing RNASeq-based, complementary TRA CDR3-mutant TP53 (black, n=11), compared with non-complementary TRA CDR3-mutant TP53 (grey, n=45). Median OS for SARC case IDs representing complementary TRA CDR3-mutant TP53, undefined; for non-complementary TRA CDR3-mutant TP53 AAs, 64.16 months. Log rank comparison p-value=0.1109.

FIG. 11 shows survival rates represented by case IDs with CDR3-mutant AA complementarity when considering the three most frequently mutated genes in the TCGA-SARC database. KM OS analysis for SARC case IDs representing RNASeq-based complementary CDR3-mutant TP53, ATRX, or RB1 (black, n=19), compared to patients with non-complementary CDR3-mutant TP53, ATRX, and RB1 (grey, n=54). Median OS for SARC case IDs representing complementary CDR3-mutant TP53, ATRX, or RB1, undefined; representing non-complementary CDR3-mutant TP53, ATRX, and RB1, 48.55 months. Log rank comparison p=0.0268.

FIG. 12 shows Kaplan-Meier analysis comparing patients with tumor TRA recoveries vs all remaining.

FIGS. 13A-13D show analysis of clinical data gathered from the TCGA provisional database that were first analyzed for OS distinctions using KM curves. Case IDs distinguished by primary pathologic length (FIG. 13A, P value=0.0011), multifocal disease (FIG. 13B, P value=0.0002), fraction of genome altered (FIG. 13C, P value=0.0014), or surgical margin (FIG. 13D, P value=0.0002), resulted in an OS distinction on univariate analysis.

FIGS. 14A and 14B show survival rates associated with complementarity scores based on TCR CDR3s recovered from SKCM tumor specimen exome files and mutant amino acids (AA). FIG. 14A shows that there is an increased rate of survival associated with the TCR CDR3-mutant AA, electrostatic charge complementarity, based on an assessment using the entire SKCM mutanome (Cox regression: p value=0.00091). For the KM analysis indicated in the figure, upper 50^{th}-percentile of TCR CDR3-mutant AA complementarity, black line. Lower 50^{th}-percentile of TCR CDR3-mutant AA complementarity, grey line (KM log rank p-value=0.0093). FIG. 14B shows that the indicated panels represent two of three genes that represent higher survival rates associated with TCR CDR3-mutant AA complementarity. The panels make survival rate comparisons with various SKCM subsets: case ID subset with BRAF mutations and TCR recombination read recovery, where the complementarity score was below zero, i.e., the most electrostatically complementary combinations (black line), median survival, 167.58 months; subset with BRAF mutations and TCR recombination read recovery, where the complementarity score was above or equal to zero, i.e., least electrostatically complementary combinations (dark grey line) median survival, 48.85 months (p value=0.0137, for first two comparisons); subset with no BRAF mutations (medium grey line), median survival: 66.69 months; subset with BRAF mutations but no recovery of TRA or TRB recombination reads (light grey line), median survival, 53.48 months. SKCM case ID subset with DNAH9 mutations and TCR read recovery, with the TCR CDR3 complementarity score below zero, median survival: 268.53 months; subset with DNAH9 mutations and TCR recombination read recovery and complementarity score above or equal to zero (dark grey line), median survival: 66.69 months (For the first two comparisons, KM log rank p-value=0.0065); subset with no DNAH9 mutations (medium grey line), median survival: 81.14 months; subset with DNAH9 mutations but with no recovery of TRB recombination reads median survival: 37.91 months.

FIGS. 15A-15C show TCR recombination reads recovered from blood WXS files represent survival rate distinctions. FIG. 15A shows SKCM survival rate represented by recovery of either TRA or TRB recombination reads from SKCM patient, blood WXS files only (black line), median survival, 94.91 months; survival rate represented by samples where there was no recovery of TCR recombination reads from blood WXS files, median survival, 65.44 months (grey line); KM log-rank p-value, 0.034. FIG. 15B shows BRCA survival rate represented by recovery of either TRA or TRB recombination reads from BRCA patient, blood WXS files only (black line), median survival, 212.1 months; survival rate represented by samples where there was no recovery of TCR recombination reads from blood WXS files, median survival (grey line), 97.4 months; KM log-rank p-value, <0.0001. FIG. 15C shows LUSC survival rate represented by recovery of either TRA or TRB recombination reads from LUSC patient, blood WXS files only (black line), median survival, 75.03 months; survival rate represented by samples where there was no recovery of TCR recombination reads from blood WXS files (grey line). KM log-rank p-value, 0.0015, median survival, 45.15 months.

FIGS. 16A-16D show survival rates associated with either high or low TCR CDR3, mutant AA complementarity, based on TCR recombination reads recovered from either tumor specimen and blood WXS files. FIG. 16A shows SKCM upper 50th-percentile, TCR CDR3-mutant AA complementarity (black line), median survival time, 133.4 months; SKCM lower 50^{th}-percentile, TCR CDR3-mutant AA complementarity (grey line), median survival time, 66.69 months (KM log-rank p-value, 0.0012; Cox regression p-value, 0.0013). FIG. 16B shows BRCA upper 50^{th}-percentile, TCR CDR3-mutant AA complementarity (black line), median survival time, 212.1 months; BRCA lower 50^{th}-percentile, TCR CDR3-mutant AA complementarity (grey line), median survival time, 129.6 months (KM log-rank p-value, 0.121; Cox regression p-value, 0.030). FIG. 16C shows CESC upper 50th-percentile, TCR CDR3-mutant AA complementarity (black line), median survival time, 107.1 months; CESC lower 50-percentile, TCR CDR3-mutant AA complementarity (grey line), median survival time, 67.41 months (KM log-rank p-value, 0.05; Cox regression p-value, 0.018). FIG. 16D shows LUSC upper 50^{th}-percentile, TCR CDR3-mutant AA complementarity (black line), median survival time, 75.03 months; LUSC lower 50^{th}-percentile, TCR CDR3-mutant AA complementarity (grey line), median survival time, 43.2 months (KM log-rank p-value, 0.01, Cox regression p-value, 0.016).

FIG. 17 shows survival rates associated with complementarity scores based on TCR CDR3s recovered from SKCM tumor specimen RNAseq files and mutant BRAF. There is an increased rate of survival associated with the TCR CDR3-mutant AA, electrostatic charge complementarity (See also FIGS. 19 and 20). Black, complementary CS, grey non-complementary CS. (KM log rank p-value=0.029).

FIG. 18 shows benchmarking NCPR-based complementarity score (CS) calculations. For the proper NCPR calculation, best NCPR CS (most negative) for either a TRA or TRB CDR3 and its cognate epitope was obtained. Then, the CDR3s originally associated with their cognate epitopes were randomly associated with other epitopes, using a random number generator, and the NCPR-based CSs were re-calculated. The randomization and re-calculations were repeated five times. For each comparison, the CSs obtained with the proper CDR3-epitope combination reflected more complementarity (were more negative) than the CSs generated by random re-calculations.

FIG. 19 shows programmatic workflow for complementarity score calculation. WXS=whole exome file; NCPR=net charge per residue; CDR3=complementarity determining region-3, for either the TRA or TRB recombination read; AA=amino acid; TCGA=the cancer genome atlas; KM=Kaplan-Meier.

FIG. 20 shows example calculation of a complementarity score for one case ID, one mutant amino acid only. NCPR=net charge per residue; CDR3=complementarity determining region-3, for either the TRA or TRB recombination read. The CDR3 sequences shown herein are the ones set forth in SEQ ID NO: 1, SEQ ID NO: 2, SEQ ID NO: 3, and SEQ ID NO. 4.

FIGS. 21A-21C show TRB CDR3-mutant IDH1 complementarity and survival rates. FIG. 21A shows KM analysis of TCGA-LGG case IDs representing complementary TRB CDR3-mutant IDH1 CSs (black line, 100 case IDs) or non-complementary CSs (grey line, 158 case IDs), where complementarity or lack of complementarity is based on the NCPR-CS calculation. FIG. 21B shows KM analysis of TCGA-SKCM case IDs representing complementary TRB CDR3-mutant NRAS CSs (black line) or non-complementary CSs (grey line), where complementarity or lack of complementarity is based on TRB CDR3 sequences obtained from SKCM tumor RNASeq files Log rank p-value=0.0019. FIG. 21C shows KM analysis of TCGA-SKCM case LDs representing complementary TRB CDR3-mutant NRAS CSs (black line) or non-complementary CSs (grey line), where complementarity or lack of complementarity is based on TRB CDR3 sequences obtained from SKCM tumor WXS files Log rank p-value=0.0368.

FIG. 22A shows KM analysis of case IDs representing complementary TRB CDR3-mutant IDH1 CSs (black line, 130 case IDs) or non-complementary CSs (grey line, 129 case IDs, where complementarity or lack of complementarity is based on the Uversky hydropathy CS and where complementary CSs are represented by the upper 50^{th} percentile of the CSs; and non-complementary CSs are represented by the lower 50^{th} percentile of the CSs.

FIG. 22B shows KM analysis of case IDs representing complementary TRB CDR3-mutant IDH1 CSs (black line, 130 case IDs) or non-complementary CSs (grey line, 129 case IDs), where complementarity or lack of complementarity is based on a combined NCPR, Uversky hydropathy CS, in turned based on averaging the z-scores for the CDR3-mutnat IDH1 CS sets for each of the two chemical parameters.

FIGS. 23A-23C show comparison of survival rates represented by non-complementary TRB CDR3-mutant IDH1 CSs versus lack of recovery of TRB recombination reads. This KM analysis compares the survival rates associated with case IDs with non-complementary TRB CDR3-mutant IDH1 CSs versus case IDs representing mutant IDH1 but where there was no recovery of TRB recombination reads. FIG. 23A shows KM analysis of TCGA-LGG case IDs representing non-complementary TRB CDR3-mutant IDH1 CSs (grey line, 158 case IDs), where lack of complementarity is based on the NCPR-CS calculation; versus case IDs that are mutant for IDH1 and where there was no recovery of TRB recombination reads (black line, 130 case IDs). FIG. 23B shows KM analysis of case IDs representing non-complementary TRB CDR3-mutant IDH1 CSs (grey line, 129 case IDs), where lack of complementarity is based on the Uversky hydropathy CS and non-complementary CSs are represented by the lower 50^{th} percentile of the Uversky hydropathy CSs; versus case IDs that are mutant for IDH1 and where there was no recovery of TRB recombination reads (black line, 130 case IDs). FIG. 23C shows KM analysis of case IDs representing non-complementary TRB CDR3-mutant IDH1 CSs (grey line, 129 case IDs), where lack of complementarity is based on a combined NCPR, Uversky hydropathy CS, as in FIG. 21C; versus case IDs that are mutant for IDH1 and where there was no recovery of TRB recombination reads (black line, 130 case IDs).

FIGS. 24A-24C show comparison of survival rates represented by complementary TRB CDR3-mutant IDH1 CSs versus lack of IDH1 mutants. FIG. 24A shows KM analysis of TCGA-LGG case IDs representing complementary TRB CDR3-mutant IDH1 CSs (black line, 100 case IDs), where complementarity is based on the NCPR-CS calculation; versus case IDs where there is not mutant IDH1 (grey line, 126 case IDs). FIG. 24B shows KM analysis of case IDs representing complementary TRB CDR3-mutant IDH1 CSs (black line, 130 case IDs), where complementarity is based on the Uversky hydropathy CS and where complementary CSs are represented by the upper 50′^{h} percentile of the CSs; versus case IDs where there is not mutant IDH1 (grey line, 126 case IDs). FIG. 24C shows KM analysis of case IDs representing complementary TRB CDR3-mutant IDH1 CSs (black line, 130 case IDs), where complementarity is based on a combined NCPR, Uversky hydropathy CS as in FIG. 21C; versus case IDs where there is not mutant IDH1 (grey line, 126 case IDs).

FIGS. 25A-25C show IGH CDR3-mutant IDH1 complementarity and survival rates. FIG. 25A shows KM analysis of case IDs representing complementary IGH CDR3-mutant IDH1 CSs (black line, 21 case IDs) or non-complementary CSs (grey line, 20 case IDs), where complementarity or lack of complementarity is based on the Uversky hydropathy CS and where complementary CSs are represented by the upper 50^{th} percentile of the CSs; and non-complementary CSs are represented by the lower 50^{th} percentile of the CSs. FIG. 25B shows KM analysis of case IDs representing non-complementary IGH CDR3-mutant IDH1 CSs (grey line, 20 case IDs), where lack of complementarity is based on the Uversky hydropathy CS and non-complementary CSs are represented by the lower 50^{th} percentile of the Uversky hydropathy CSs; versus case IDs that are mutant for IDH1 and where there was no recovery of IGH recombination reads (black line, 388 case IDs). FIG. 25C shows KM analysis of case IDs representing complementary IGH CDR3-mutant IDH1 CSs (black line, 21 case IDs), where complementarity is based on the Uversky hydropathy CS and where complementary CSs are represented by the upper 50^{th} percentile of the CSs; versus case IDs where there is not mutant IDH1 (grey line, 126 case IDs).

FIG. 26 shows overall survival for complementary TRB CDR3-mutant IDH case IDs (red) versus all remaining samples (blue). This figure was obtained from cBioPortal.org after inputting the case IDs indicated in FIG. 21A.

FIG. 27 shows overall survival for complementary TRB CDR3-mutant IDH case IDs (red) versus all remaining samples (blue). This figure was obtained from cBioPortal.org after inputting the case IDs indicated in FIG. 22A.

FIG. 28 shows heat map for tissue specificity of the apoptosis effector genes tested. AIFM3 indicates a very high level of brain specific expression. The RNASeq values were obtained from GTE x Portal. (www.gtexportal.org/home/). The values used to generate this heat map were normalized to whole blood RNAseq values. Several apoptosis genes are not included because only zero values were available for every tissue. The display (heat map) was generated by the authors using the numerical values as indicated above. The Y-axis tissue list, lists every other tissue in the analysis.

FIG. 29 shows overall survival for SKCM case IDs representing complementary (versus noncomplementary) TRA CDR3-mutant BRAF NCPR CSs. Log rank p-value=0.0244. Complementary case IDs, 179; median survival, 111.01 months. Noncomplementary case IDs, 35; median survival, 46.78 months. TRA CDR3s represent TRA recombination reads recovered from both tumor and blood WXS files.

FIGS. 30A-30F show Kaplan-Meier (KM) DFS analyses for TCGA-BRCA case IDs representing BCR CDR3-mutant TP53 combinations. FIG. 30A shows comparison of the DFS rates for case IDs representing complementary (black) versus noncomplementary (gray) BCR CDR3-TP53 mutant combinations, using the extended approach (p-value=0.0292). FIG. 30B shows comparison of DFS rates for case IDs representing complementary (black) versus noncomplementary (gray) BCR CDR3-TP53 mutant combinations, using the abbreviated approach (p-value=0.0126). FIG. 30C shows comparison of DFS rates for case IDs representing complementary (black) BCR CDR3-TP53 mutant combinations, using the abbreviated approach, versus all remaining (gray) case IDs (p-value=0.0330). FIG. 30D shows box and whisker plots of RNASeq values used to compare gene expression for BRCA tumor samples representing complementary and noncomplementary CDR3-mutant TP53 combinations. The means for the RNASeq values for genes being compared for complementary (black) and noncomplementary (gray) tumor samples using the extended approach are as follows: CD19 (i): complementary (mean, 145.95); noncomplementary (mean, 64.77). CD22 (ii): complementary (369.96); noncomplementary (194.42). CD72 (iii): complementary (182.02); noncomplementary (132.78). CD79A (iv): complementary (1004. 24); noncomplementary (540.59). CD79B (v): complementary (336.96); noncomplementary (206.87). MS4A1 (vi): complementary (672.25); noncomplementary (325.24). TNFRSF13B (vii): complementary (38.50); noncomplementary (19.01). TNFRSF13C (viii): complementary (21.18); noncomplementary (12.29). TNFRSFI7 (ix): complementary (137.95); noncomplementary (80.64). FIG. 30E shows KM DFS analysis of ovarian cancer (OV) case IDs based on recovery of BCR CDR3s from OV WXS files. Comparison of DFS for case IDs representing recovery of IGH, IGK or IGL BCR CDR3s (n=29), versus all remaining case IDs (n=470; p-value=0.022). FIG. 30F shows KM DFS analysis based on physico-chemical properties of OV, WXS-based BCR CDR3s. Comparison of DFS between case IDs from IGH and IGL top half of tumor samples with CDR3 AA sequences representative of higher aromaticity (n=10) versus bottom half (n=9) of tumor samples with CDR3 AA sequences representative of lower aromaticity (p-value=0.0176).

FIG. 31 shows comparison of DFS rates for case IDs representing noncomplementary (black) BCR CDR3-TP53 mutant combinations using the abbreviated approach versus all remaining (gray) case IDs (p-value=0.2396).

FIG. 32 shows comparison of complementary (black) vs all remaining case IDs (grey) using the extended approach, P-value=0.078.

FIG. 33 shows comparison of noncomplementary (black) vs all remaining case IDs (grey) using the extended approach, P-value=0.3124.

FIG. 34 shows survival rates based on complementary scores between TCR CDR3-MACF1 electrostatic complementarity recovered from BLCA tumor exome files and mutant AA sequences. The KM curve presented in the figure illustrates the overall survival for patients with electrostatic complementarity (solid line), those with non-complementary mutants (dotted line), and the overall BLCA patient cohort found in the TCGA (dot-and-dashed line). TCR CDR3-MACF1 mutants with electrostatic complementarity did not reach a median OS as >50% of patients are still alive, median OS was 19.35 months for non-complementary TCR CDR3-MACF1 mutants, and 35.38 months in the overall BLCA group. OS was prolonged in the group with electrostatic complementary versus patients with non-complementary mutant (p=0.007) and the overall BLCA population (p=0.013). Non-complementary TCR CDR3-MACF1 mutants survival was similar to the overall BLCA patients (p=0.233).

FIG. 35 shows survival rates based on complementary scores between TCR CDR3-MACF1 electrostatic complementarity recovered from BLCA tumor exome files and mutant AA sequences. The figure represents the KM curve for DFS. The median DFS for TCR CDR3-MACF1 mutants with electrostatic complementarity cannot be accurately calculated as >50% have not relapsed vs 37.78 months for those with non-complementary mutants (p=0.016) vs 28.29 months for the entire BLCA group (p=0.003).

### DETAILED DESCRIPTION

The anti-tumor immune response is considered to be due to the tumor infiltrating lymphocytes that bind to tumor antigens, which can be either wild-type, early stem cell proteins, presumably foreign to a developed immune system; or mutant peptides, foreign to the immune system because of a mutant amino acid or an otherwise somatically altered amino acid sequence. Disclosed herein are novel methods for assessing the complementarity of tumor mutant peptides and complementarity determining regions (CDRs) of T cell receptors, B cell receptors, and antibodies, based on the retrieval of CDR3 amino acid sequences from both tumor specimen and patient blood exomes and by using a process of assessing CDR3s and mutant amino acid electrical charges. It is shown herein that high electrostatic complementarity and hydropathy values are associated with higher survival rates. In addition, the approach shown herein leads to the identification of genes contributing significantly to the complementary, TCR CDR3, mutant amino acids. The data shown herein indicate a novel approach to tumor immunoscoring and uses thereof for diagnosing, monitoring, and treating cancers. These methods are also used for the identification of high priority neo-antigen, peptide vaccines for treating cancers and/or to the identification of ex vivo stimulants of tumor infiltrating lymphocytes.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and a second polynucleotide encoding a protein associated with the cancer;
determining a complementarity score of the CDR domain and the protein associated with the cancer, comprising multiplying a net charge per residue (NCPR) of the CDR domain by a value of change in charge due to an amino acid substitution in the protein associated with the cancer and further by “−1”;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a complementarity score of the CDR domain and the protein associated with the cancer, comprising multiplying a Uversky hydropathy score of the CDR domain by a value of change in Uversky hydropathy score due to an amino acid substitution in the protein associated with the cancer;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.

In some aspects, disclosed herein is a method for predicting an overall survival of a subject having a cancer, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer;
determining a mean z-score by averaging a first z-score of the first complementarity score and a second z-score of the second complementarity score, wherein the first z-score and the second z-score are relative to a reference database;
and
predicting one of:
   a. the subject as having a shorter overall survival if the mean z-score is lower in the biological sample derived from the subject compared to a reference control, or
   b. the subject as having a longer overall survival if the mean z-score is higher in the biological sample derived from the subject compared to a reference control.

In some aspects, disclosed herein is a method for screening an antigen-binding protein for treating a cancer, comprising:
obtaining a first sequence of a CDR domain of the antigen-binding protein and a second sequence of a protein associated with the cancer;
determining a complementarity score of the CDR domain and the protein associated with the cancer; and
determining the antigen-binding protein for treating the cancer if the complementarity score thereof is higher compared to a reference control,
wherein the complementarity score of the CDR domain of the antigen-binding protein and the protein associated with the cancer is determined by:
   multiplying a net charge per residue (NCPR) of the CDR domain by a value of change in charge due to an amino acid substitution in the protein associated with the cancer and further by “−1”;
   multiplying a Uversky hydropathy score of the CDR domain by a value of change in Uversky hydropathy score due to an amino acid substitution in the protein associated with the cancer; or
   determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
   determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer; and
   determining a mean z-score by averaging a first z-score of the first complementarity score and a second z-score of the second complementarity score, wherein the first z-score and the second z-score are relative to a reference database; and
   wherein the complementarity score is equal to the mean z-score.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “about” and “approximately” are defined as being “'close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). “Complementarity Determining Regions” or “CDRs” refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. CDR3s are most variable, of which the tertiary structure determines antigen recognition of an antibody. Framework Regions (FRs) refer to amino acid sequences interposed between CDRs.

As used herein, the term “antibody or a functional fragment thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain antigen recognition property are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or functional fragments thereof” are conjugates of antibody fragments and antigen binding proteins (single chain antibodies). The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

“B cell receptor” or “BCR” refers to a membrane-bound immunoglobulin on B cell surface that forms a type 1 transmembrane receptor protein usually located on the outer surface of a B cell. B cell receptor is almost identical in structure to antibody except the C-terminal region of a B cell receptor heavy chain comprising a short hydrophobic stretch that spans the plasma membrane.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “cancer” or “neoplasms” used herein meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as malignancies affecting skin, brain, spinal cord, cervix, bladder, lung, breast, thyroid, lymphoid tissues, connecting tissues, gastrointestinal, and genito-urinary tracts, that include, but are not limited to, glioma, melanoma, lung cancer, breast cancer, cervical squamous cell carcinoma, bladder cancer, and soft tissue sarcoma. The term “cancer metastasis” has its general meaning in the art and refers to the spread of a tumor from one organ or part to another non-adjacent organ or part.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Complementarity Determining Regions” or “CDRs” refer to amino acid sequences which together define the binding affinity and specificity of the natural variable domain of a native binding site of an antibody, a BCR, or a TCR. The extent of CDRs have been precisely defined and identified by methods known in the arts, such as “Sequences of Proteins of Immunological Interest,” E. Kabat et al., U.S. Department of Health and Human Services, (1991); Wu T T, Kabat E A. “An analysis of the sequences of the variable regions of Bence Jones proteins and myeloma light chains and their implications for antibody complementarity”. J Exp Med (1970); “Canonical structures for the hypervariable regions of immunoglobulins”. Chothia C, Lesk A M J Mol Biol. 1987 Aug. 20; 196(4):901-17, which are incorporated herein by reference for all purposes. In some embodiments, a CDR begins by the second cysteine in the variable domain, and at the end by the first amino acid in the conserved Phe/Trp-Gly-X-Gly J-region motif.

A “composition” is intended to include a combination of active agent and another compound or composition, inert (for example, a detectable agent or label) or active, such as an adjuvant.

As used herein, the terms “determining,” “measuring,” and “assessing,” and “assaying” are used interchangeably and include both quantitative and qualitative determinations.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein the term “encoding” refers to the inherent property of specific sequences of nucleotides in a nucleic acid, to serve as templates for synthesis of other molecules having a defined sequence of nucleotides (i.e. rRNA, tRNA, other RNA molecules) or amino acids and the biological properties resulting therefrom.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as antigen binding and antigen recognition.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “isolating” as used herein refers to isolation from a biological sample, i.e., blood, plasma, tissues, exosomes, or cells. As used herein the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mot. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

The term “specific binding” refers to the ability of an antigen-binding protein (e.g., an antibody) to preferentially bind to a particular analyte that is present in a homogeneous mixture of different analytes. In certain embodiments, a specific binding interaction will discriminate between desirable and undesirable antigen in a sample, in some embodiments more than about 10 to 100-fold or more (e.g., more than about 1000- or 10,000-fold). In certain embodiments, the affinity between an antigen-binding protein (e.g., an antibody, TCR, or BCR) and an antigen when they are specifically bound in an antigen-binding protein/antigen complex is characterized by a KD (dissociation constant) of less than 10-6 M, less than 10-7 M, less than 10-8 M, less than 10-9 M, less than 10-9 M, less than 10-11 M, or less than about 10-12 M.

The term “subject” or “host” refers to any individual who is the target of administration or treatment. The subject can be a vertebrate, for example, a mammal. Thus, the subject can be a human or veterinary patient. The term “patient” refers to a subject under the treatment of a clinician, e.g., physician. The subject can be either male or female.

“T cell receptor” or “TCR” refers to an immunoglobulin superfamily member (having a variable binding domain, a constant domain, a transmembrane region, and a short cytoplasmic tail; see, e.g., Janeway et al., Immunobiology: The Immune System in Health and Disease, 3rd Ed., Current Biology Publications, p. 4:33, 1997) capable of specifically binding to an antigen peptide bound to a major histocompatibility complex (MHC). A TCR can be found on the surface of a cell and generally is comprised of a heterodimer having α and β chains (also known as TCRα and TCRβ, respectively), or γ and δ chains (also known as TCRγ and TCRδ of a γδTCR, respectively). Like immunoglobulins, the extracellular portion of TCR chains (e.g., α-chain, β-chain) contain two immunoglobulin domains, a variable domain (e.g., α-chain variable domain or Va, β-chain variable domain or Vb) at the N-terminus, and one constant domain (e.g., α-chain constant domain or Ca, β-chain constant domain or Cb) adjacent to the cell membrane. Also, like immunoglobulins, the variable domains contain complementary determining regions (CDRs) separated by framework regions (FRs). For an αβTCR, the alpha chain and beta chain each have three CDRs, wherein CDR3 is the most variable of which the tertiary structure determines antigen recognition. The source of a TCR as used in the present disclosure may be from various animal species, such as a human, mouse, rat, rabbit or other mammal.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder (e.g., a cancer), or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition (e.g. cancer). Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods of Diagnosing and Treating Cancers

In some aspects, disclosed herein is a method for predicting the overall survival of a subject having a cancer, comprising:
isolating a nucleic acid from a biological sample derived from the subject;
sequencing a first polynucleotide encoding a complementarity determining region (CDR) domain of an antigen-binding protein and a second polynucleotide encoding a protein associated with the cancer;
determining a complementarity score of the CDR domain and the protein associated with the cancer, comprising multiplying a net charge per residue (NCPR) of the CDR domain by a value of change in charge due to an amino acid substitution in the protein associated with the cancer and further by “−1”;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.

It should be understood and contemplated herein that “antigen recognition” refers to an affinity achieved through noncovalent interactions between a TCR and an antigen-MHC complex or between an antibody/BCR and an antigen. Such noncovalent interactions can be, for example hydrophobic interactions, electrophilic interactions, or van der Waals forces. Accordingly, “antigen-binding protein” refers to a protein can bind to an antigen through antigen recognition. The antigen-binding protein disclosed herein can be, for example, a TCR, a BCR, or an antibody, or a functional fragment thereof. In some embodiments, the antigen-binding protein is a TCR or a functional fragment thereof. In some embodiments, the antigen-binding protein is TCR alpha chain or a functional fragment thereof. In some embodiments, the antigen-binding protein is TCR beta chain or a functional fragment thereof. In some embodiments, the antigen-binding protein is TCRγ or a functional fragment thereof. In some embodiments, the antigen-binding protein is TCRδ or a functional fragment thereof.

In some embodiments, the antigen-binding protein is a BCR or an antibody or a functional fragment thereof. In some embodiments, the antigen-binding protein is a BCR or a functional fragment thereof. In some embodiments, the antigen-binding protein is an antibody or a functional fragment thereof. As noted above, the isotype of an antibody or a BCR is can be IgM, IgD, IgG, IgA, and IgE. Accordingly, in some embodiments, the antigen-binding protein is an IgG antibody or a functional fragment thereof. In some embodiments, the antigen-binding protein is an IgG BCR or a functional fragment thereof. In some embodiments, the antigen-binding protein is a light chain of an IgG antibody or a functional fragment thereof. In some embodiments, the antigen-binding protein is a heavy chain of an IgG antibody or a functional fragment thereof. In some embodiment, antigen-binding protein is a light chain of IgG BCR or a functional fragment thereof. In some embodiments, the antigen-binding protein is a heavy chain of IgG BCR or a functional fragment thereof.

As noted above, “Complementarity Determining Regions” or “CDRs” refer to amino acid sequences which together define the binding affinity and specificity of the natural Fv region of a native binding site of an immunoglobulin or a TCR. The light (L) and heavy (H) chains of an immunoglobulin each have three CDRs, designated L-CDR1, L-CDR2, L-CDR3 and H-CDR1, H-CDR2, H-CDR3, respectively. For an αβTCR, the alpha chain and beta chain each have three CDRs. Accordingly, in some embodiments, the CDR is a CDR1 of a light chain of an antibody. In some embodiments, the CDR is a CDR2 of a light chain of an antibody. In some embodiments, the CDR is a CDR3 of a light chain of an antibody. In some embodiments, the CDR is a CDR1 of a heavy chain of an antibody. In some embodiments, the CDR is a CDR2 of a heavy chain of an antibody. In some embodiments, the CDR is a CDR3 of a heavy chain of an antibody. In some embodiments the antibody of any preceding aspects is selected from the group consisting of IgM, IgD, IgG, IgA, and IgE.

In some embodiments, the CDR is a CDR1 of a light chain of a BCR. In some embodiments, the CDR is a CDR2 of a light chain of a BCR. In some embodiments, the CDR is a CDR3 of a light chain of a BCR. In some embodiments, the CDR is a CDR1 of a heavy chain of a BCR. In some embodiments, the CDR is a CDR2 of a heavy chain of a BCR. In some embodiments, the CDR is a CDR3 of a heavy chain of a BCR. In some embodiments the BCR of any preceding aspects is selected from the group consisting of IgM, IgD, IgG, IgA, and IgE.

In some embodiments, the CDR is a CDR1 of an alpha chain of a TCR. In some embodiments, the CDR is a CDR2 of an alpha chain of a TCR. In some embodiments, the CDR is a CDR3 of an alpha chain of a TCR. In some embodiments, the CDR is a CDR1 of a beta chain of a TCR. In some embodiments, the CDR is a CDR2 of a beta chain of a TCR. In some embodiments, the CDR is a CDR3 of a beta chain of a TCR.

In some embodiments, disclosed herein is a method for predicting the CDR is a CDR of a γ chain of a γδTCR. In some embodiments, the CDR is a CDR of a δ chain of a γδTCR.

In some embodiments, the nucleic acid is any preceding aspect is a DNA or an RNA. In some embodiments, the nucleic acid is a DNA. In some embodiments, the nucleic acid is an RNA. In some embodiments, the polynucleotide is a DNA. In some embodiments, the polynucleotide is an RNA. In some embodiments, the DNA comprises an exon and an intron. In some embodiments, the DNA is an exon.

It should be understood and herein contemplated that a polypeptide's net charge depends on the number of the charged amino acids the polypeptide contains and the pH of the environment. At physiological pH (pH 7.4), for example, five amino acid residues out of the 20 common amino acids can be charged: two are negative charged: aspartic acid (Asp, D) and glutamic acid (Glu, E) (acidic side chains), and three are positive charged: lysine (Lys, K), arginine (Arg, R) and histidine (His, H) (basic side chains). The term “net charge per residue” of a polypeptide (e.g. a CDR domain) in a certain pH environment is calculated as dividing the overall charge of the polypeptide in such pH environment by the number of amino acid residues of the polypeptide.

In some embodiments, a CDR begins by a second cysteine in the variable domain of an antigen-binding protein, and at the end by the first amino acid in the conserved Phe/Trp-Gly-X-Gly J-region motif, is analyzed for net charge per residue (NCPR) using the localCIDER python package (pappulab.github.io/localCIDER/.)

Properties of the amino acids found in proteins

| | pK [symbol] of | pK [symbol] of | pK [symbol] of |
|---|---|---|---|
| | α-Carboxyl | α-Amino | Ionizing |
| Name | Group | Group | Side Chain [symbol] |
| Alanine | 2.3 | 9.7 | — |
| Arginine | 2.2 | 9.0 | 12.5 |
| Asparagine | 2.0 | 8.8 | — |
| Aspartic acid | 2.1 | 9.8 | 3.9 |
| Cysteine | 1.8 | 10.8 | 8.3 |
| Glutamine | 2.2 | 9.1 | — |
| Glutamic acid | 2.2 | 9.7 | 4.2 |
| Glycine | 2.3 | 9.6 | — |
| Histidine | 1.8 | 9.2 | 6.0 |
| Isoleucine | 2.4 | 9.7 | — |
| Leucine | 2.4 | 9.6 | — |
| Lysine | 2.2 | 9.0 | 10.0 |
| Methionine | 2.3 | 9.2 | — |
| Phenylalanine | 1.8 | 9.1 | — |
| Proline | 2.0 | 10.6 | — |
| Serine | 2.2 | 9.2 | — |
| Threonine | 2.6 | 10.4 | — |
| Tryptophan | 2.4 | 9.4 | — |
| Tyrosine | 2.2 | 9.1 | 10.1 |
| Valine | 2.3 | 9.6 | — |

[symbol]

indicates data missing or illegible when filed

As noted above, a complementarity score of a CDR domain and a protein associated with a cancer can be determined by multiplying a net charge per residue (NCPR) of the CDR domain by a value of change in charge due to an amino acid substitution in the protein associated with the cancer and further by “−1”. For example, an R->H mutation in a protein associated with a cancer (e.g., LGG) yields a −1.0 net change in charge at physiological pH, which if multiplied by a CDR3 domain NCPR of +0.5 and then by −1 yields a complementarity score of +0.5.

In some embodiments, a complementarity score that is higher than 0 denotes the subject as having a longer overall survival. Accordingly, in some embodiments, the subject has a longer overall survival if the complementarity score is higher than 0. In some embodiments, the subject has a shorter overall survival if the complementarity score is less than 0.

In some embodiments, the complementarity score can be determined by multiplying a NCPR of a CDR domain by a value of change in charge due to an amino acid substitution in a protein associated with a cancer. In some embodiments, the complementarity score can be determined by multiplying one or more NCPRs of one or more CDR domains by one or more values of change in charge due to one or more amino acid substitutions in one or more proteins associated with a cancer.

In some embodiments, the reference control is the complementarity score of a CDR domain and a protein associated with a cancer for the lowest 10%, 20%, 30%, 40%, or 50% of the complementarity score of a reference population of patient samples having the cancer. In some embodiments, the reference control is the complementarity score of a CDR domain and a protein associated with a cancer for the highest 10%, 20%, 30%, 40%, or 50% of the complementarity score of a reference population of patient samples having the cancer. Accordingly, the subject has a shorter overall survival if the complementarity score is lower in the biological sample derived from the subject compared to the reference control, and the subject has a longer overall survival if the complementarity score is higher in the biological sample derived from the subject compared to a reference control.

In some aspects, a complementarity score of a CDR domain and a protein associated with a cancer can also be determined by multiplying a net charge per residue (NCPR) of the CDR domain by a value of change in charge due to an amino acid substitution in the protein associated with the cancer. For example, an R->H mutation in a protein associated with a cancer (e.g., IGG) yields a −1.0 net change in charge at physiological pH, which if multiplied by a CDR3 domain NCPR of +0.5 yields a complementarity score of −0.5.

Accordingly, in some embodiments, a complementarity score that is less than 0 denotes the subject as having a longer overall survival. Accordingly, in some embodiments, the subject has a longer overall survival if the complementarity score is less than 0. In some embodiments, the subject has a shorter overall survival if the complementarity score is more than 0. In some embodiments, the reference control is the complementarity score of a CDR domain and a protein associated with a cancer for the lowest 10%, 20%, 30%, 40%, or 50% of the complementarity score of a reference population of patient samples having the cancer. In some embodiments, the reference control is the complementarity score of a CDR domain and a protein associated with a cancer for the highest 10%, 20%, 30%, 40%, or 50% of the complementarity score of a reference population of patient samples having the cancer. Accordingly, the subject has a longer overall survival if the complementarity score is lower in the biological sample derived from the subject compared to the reference control, and the subject has a shorter overall survival if the complementarity score is higher in the biological sample derived from the subject compared to a reference control.

In some aspects, disclosed herein is a method for predicting the overall survival of a subject having a cancer, comprising:
isolating a nucleic acid from a biological sample derived from the subject;
sequencing a first gene encoding a complementarity determining region (CDR) domain of an antigen-binding protein and second gene encoding a protein associated with the cancer;
determining a complementarity score of the CDR domain and the protein associated with the cancer, comprising multiplying a Uversky hydropathy score of the CDR domain by a value of change in Uversky hydropathy score due to an amino acid substitution in the protein associated with the cancer;
and
predicting one of:
   a. the subject as having a shorter overall survival if the complementarity score is lower in the biological sample derived from the subject compared to a reference control, or
   b. the subject as having a longer overall survival if the complementarity score is higher in the biological sample derived from the subject compared to a reference control.
      As stated above, antigen recognition of an antigen-binding protein can involve noncovalent interactions such as hydrophobic interactions. Therefore, to assess hydrophobic interactions, a complementarity score can be calculated based on Uversky hydropathy values. The present disclosure shows that a Uversky hydropathy value of a CDR domain of an antigen-binding protein can be multiplied by the Uversky hydropathy of a mutated amino acid of protein associated with a cancer. For example, an R->H mutation in the protein associated with a cancer yields a +0.144 change in Uversky hydropathy, which if multiplied by a CDR Uversky hydropathy of +0.1, yields a complementarity score of +0.0144. The Uversky Hydropathy values for amino acid residues are shown below:

| Amino Acid Residue | Uversky Hydropathy Value |
|---|---|
| A | 0.70 |
| R | 0.00 |
| N | 0.111111111 |
| D | 0.111111111 |
| C | 0.777777778 |
| E | 0.111111111 |
| Q | 0.111111111 |
| G | 0.455555556 |
| H | 0.144444444 |
| I | 0.00 |
| L | 0.922222222 |
| K | 0.066666667 |
| M | 0.711111111 |
| F | 0.811111111 |
| P | 0.322222222 |
| S | 0.411111111 |
| T | 0.422222222 |
| W | 0.40 |
| Y | 0.355555556 |
| V | 0.966666667 |

In some embodiments, a complementarity score that is higher than about, for example, about −2.00, −1.90, −1.80, −1.70, −1.60, −1.50, −1.40, −1.30, −1.20, −1.10, −1.00, −0.90, −0.80, −0.70, −0.60, −0.50, −0.40, −0.30, −0.20, −0.10, 0, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.50, 4.00, 4.50 or 5.00 denotes the subject as having a longer overall survival. In some embodiments, a complementarity score that is higher than about 0 denotes the subject as having a longer overall survival. Accordingly, in some embodiments, the subject as having a longer overall survival if the complementarity score is higher than 0. In some embodiments, the subject as having a shorter overall survival if the complementarity score is less than 0.

In some embodiments, the reference control is the complementarity score of a CDR domain and a protein associated with a cancer for the lowest 10%, 20%, 30%, 40%, or 50% of the complementarity score of a reference population of patient samples having the cancer. In some embodiments, the reference control is the complementarity score of a CDR domain and a protein associated with a cancer for the highest 10%, 20%, 30%, 40%, or 50% of the complementarity score of a reference population of patient samples having the cancer. Accordingly, the subject has a shorter overall survival if the complementarity score is lower in the biological sample derived from the subject compared to the reference control, and the subject has a longer overall survival if the complementarity score is higher in the biological sample derived from the subject compared to a reference control.

In some aspects, disclosed herein is a method for predicting the overall survival of a subject having a cancer, comprising:
isolating a nucleic acid from a biological sample derived from the subject;
sequencing a first gene encoding a complementarity determining region (CDR) domain of an antigen-binding protein and second gene encoding a protein associated with the cancer;
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer;
determining a mean z-score by averaging a first z-score of the first complementarity score and a second z-score of the second complementarity score, wherein the first z-score and the second z-score are relative to a reference database;
and
predicting one of:
   a. the subject as having a shorter overall survival if the mean z-score is lower in the biological sample derived from the subject compared to a reference control, or
   b. the subject as having a longer overall survival if the mean z-score is higher in the biological sample derived from the subject compared to a reference control.

In some embodiments, a mean z-score that is higher than about, for example, about −2.00, −1.50, −1.00, −0.50, 0, 0.10, 0.20, 0.30, 0.40, 0.50, 0.60, 0.70, 0.80, 0.90, 1.00, 1.10, 1.20, 1.30, 1.40, 1.50, 1.60, 1.70, 1.80, 1.90, 2.00, 2.10, 2.20, 2.30, 2.40, 2.50, 2.60, 2.70, 2.80, 2.90, 3.00, 3.10, 3.20, 3.30, 3.40, 3.50, 3.60, 3.70, 3.80, 3.90, 4.00, 4.10, 4.20, 4.30, 4.40, 4.50, 4.60, 4.70, 4.80, 4.90 or 5.00 denotes the subject as having a longer overall survival. In some embodiments, a mean z-score that is higher than 0 indicates the subject as having a longer overall survival. Accordingly, in some embodiments, the subject as having a longer overall survival if the mean z-score is higher than 0. In some embodiments, the subject as having a shorter overall survival if the mean z-score is less than 0.

In some embodiments, the reference control is the z-score of a CDR domain and a protein associated with a cancer for the lowest 10%, 20%, 30%, 40%, or 50% relative to a reference database of patient samples having the cancer. In some embodiments, the reference control is the complementarity score of a CDR domain and a protein associated with a cancer for the highest 10%, 20%, 30%, 40%, or 50% relative to a reference database of patient samples having the cancer. Accordingly, the subject has a shorter overall survival if the mean z-score is lower in the biological sample derived from the subject compared to the reference control, and the subject has a longer overall survival if the mean z-score is higher in the biological sample derived from the subject compared to a reference control.

In some embodiments, the subject as having a shorter overall survival comprises the subject having an overall survival of about 1 month or less, about 2 months or less, about 4 months or less, about 6 months or less, about 8 months or less, about 10 months or less, about 12 months or less, about 14 months or less, about 16 months or less, about 18 months or less, about 20 months or less, about 22 months or less, about 25 months or less, or about 30 months or less, about 35 months or less, about 40 months or less, about 45 months or less, about 50 months or less, about 55 months or less, about 60 months or less, about 65 months or less, about 70 months or less, about 75 months or less, about 80 months or less, about 85 months or less, about 90 months or less, about 95 months or less, about 100 months or less, about 150 months or less, about 200 months or less, or about 250 months or less.

In some embodiments, the subject is a human. In some embodiments, the human has or is suspected of having a cancer. In some embodiments, the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

The term “protein associated with a cancer” of any preceding aspect refers to a protein that has the potential to cause cancer. In tumor cells, the gene encoding such a protein may be mutated, overly expressed, or underly expressed. This term can also refer to a protein that is mutated, overly expressed, or underly expressed in a cancer cell or a cell undergoing oncogenic processes. In some embodiments, the protein associated with a cancer is selected from the group consisting of isocitrate dehydrogenase 1 (IDH1) (External Ids: HGNC: 5382; Entrez Gene: 3417; Ensembl: ENSG00000138413; OMIM: 147700; UniProtKB: 075874), Phosphatidylinositol-4,5-Bisphosphate 3-Kinase Catalytic Subunit Alpha (PIK3CA, External Ids: HGNC: 8975; Entrez Gene: 5290; Ensembl: ENSG00000121879; OMIM: 171834; UniProtKB: P42336), Inositol 1,4,5-trisphosphate receptor, type 2 (ITPR2, External Ids: HGNC: 6181; Entrez Gene: 3709; Ensembl: ENSG00000123104; OMIM: 600144; UniProtKB: Q14571), B-Raf proto-oncogene (BRAF, External Ids: HGNC: 1097; Entrez Gene: 673; Ensembl: ENSG00000157764; OMIM: 164757; UniProtKB: P15056), Dynein heavy chain 9 (DNAH9, External Ids: HGNC: 2953; Entrez Gene: 1770; Ensembl: ENSG00000007174; OMIM: 603330; UniProtKB: Q9NYC9), myosin heavy chain 1 (MYH1, External Ids: HGNC: 7567; Entrez Gene: 4619; Ensembl: ENSG00000109061; OMIM: 160730; UniProtKB: P12882), Tenascin-R (TNR, External Ids: HGNC: 11953; Entrez Gene: 7143; Ensembl: ENSG00000116147; OMIM: 601995; UniProtKB: Q92752), Teneurin-1 (TNM1; HGNC: 8117; Entrez Gene: 10178; Ensembl: ENSG00000009694; OMIM: 300588; UniProtKB: Q9UKZ4), Plexin-A4 (PLXNA4A, External Ids: HGNC: 9102; Entrez Gene: 91584; Ensembl: ENSG00000221866; OMIM: 604280; UniProtKB: Q9HCM2), Microtubule-actin cross-linking factor 1 (MACF, External Ids: HGNC: 13664; Entrez Gene: 23499; Ensembl: ENSG00000127603; OMIM: 608271; UniProtKB: Q9UPN3), Tumor protein p53 (TP53, External Ids: HGNC: 11998; Entrez Gene: 7157; Ensembl: ENSG00000141510; OMIM: 191170; UniProtKB: P04637), ATP-dependent helicase ATRX (ATRX, External Ids; HGNC: 886; Entrez Gene: 546; Ensembl: ENSG00000085224; OMIM: 300032; UniProtKB: P46100), Neuroblastoma RAS viral oncogene homolog (NRAS, External Ids: HGNC: 7989; Entrez Gene: 4893; Ensembl: ENSG00000213281; OMIM: 164790; UniProtKB: P01111) and Retinoblastoma protein (RB1, External Ids: HGNC: 9884; Entrez Gene: 5925; Ensembl: ENSG00000139687; OMIM: 614041; UniProtKB: P06400), or a functional fragment thereof.

In some embodiments, IDH1 is the protein associated with LGG. Accordingly, disclosed herein are methods of predicting an overall survival of a subject having LGG, comprising determining a complementarity score of a CDR domain of any preceding aspects and LDH1 or a functional fragment thereof.

In some embodiments, the protein associated with a cancer is selected from the group consisting of PIK3CA and ITPR2, wherein the cancer is bladder cancer. Accordingly, disclosed herein are methods of predicting an overall survival of a subject having bladder cancer, comprising determining a complementarity score of a CDR domain of any preceding aspects and PIKCA and/or ITPR2 or a functional fragment thereof.

In some embodiments, disclosed herein are methods of predicting an overall survival of a subject having esophageal cancer, comprising determining a complementarity score of a CDR domain of any preceding aspects and a protein associated with esophageal cancer or a functional fragment thereof, wherein the protein associated with esophageal cancer is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

In some embodiments, disclosed herein are methods of predicting an overall survival of a subject having cervical squamous cell carcinoma, comprising determining a complementarity score of a CDR domain of any preceding aspects and a protein associated with cervical squamous cell carcinoma or a functional fragment thereof, wherein the protein associated with cervical squamous cell carcinoma is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

In some embodiments, the protein associated with a cancer is TP53, wherein the cancer is breast cancer. Accordingly, disclosed herein are methods of predicting an overall survival of a subject having breast cancer, comprising determining a complementarity score of a CDR domain of any preceding aspects and TP53 or a functional fragment thereof.

In some embodiments, the proteins associated with a cancer is selected from the group consisting of NRAS, BRAF, DNAH9, MYH1, and TNR, wherein the cancer is melanoma (e.g., skin cutaneous melanoma). Accordingly, disclosed herein are methods of predicting an overall survival of a subject having melanoma (e.g., skin cutaneous melanoma), comprising determining a complementarity score of a CDR domain of any preceding aspects and one or more proteins associated melanoma selected from the group consisting of NRAS, BRAF, DNAH9, MYH1, and TNR, or a functional fragment thereof.

In some embodiments, the proteins associated with a cancer is selected from the group consisting of AHNAK and ADATS, wherein the cancer is lung adenocarcinoma. Accordingly, disclosed herein are methods of predicting an overall survival of a subject having lung adenocarcinoma, comprising determining a complementarity score of a CDR domain of any preceding aspects and AHNAK and/or ADATS, or a functional fragment thereof.

In some embodiments, the proteins associated with a cancer is selected from the group consisting of TENM1 and PLXNA4, wherein the cancer is lung squamous cell carcinoma. Accordingly, disclosed herein are methods of predicting an overall survival of a subject having lung squamous cell carcinoma, comprising determining a complementarity score of a CDR domain of any preceding aspects and TENM1 and/or PLXNA4, or a functional fragment thereof.

In some embodiments, the proteins associated with a cancer is selected from the group consisting of MACF1 and PIK3CA, wherein the cancer is muscle invasive bladder cancer. Accordingly, disclosed herein are methods of predicting an overall survival of a subject having muscle invasive bladder cancer, comprising determining a complementarity score of a CDR domain of any preceding aspects and MACF1 and/or PIK3CA, or a functional fragment thereof.

In some embodiments, the proteins associated with a cancer is selected from the group consisting of TP53, ATRX, and RB1, wherein the cancer is soft tissue sarcoma. Accordingly, disclosed herein are methods of predicting an overall survival of a subject having soft tissue sarcoma, comprising determining a complementarity score of a CDR domain of any preceding aspects and a protein associated with soft tissue sarcoma that is selected from the group consisting of TP53, ATRX, and RB1, or a functional fragment thereof.

In some embodiments, the method further comprises administering to the subject an anti-cancer agent based on the prediction of the subject as having a shorter overall survival. In some embodiments, the method further comprises administering an appropriate anti-cancer agent to the subject based on the prediction of the subject as having an overall survival of about 1 month or less, about 2 months or less, about 4 months or less, about 6 months or less, about 8 months or less, about 10 months or less, about 12 months or less, about 14 months or less, about 16 months or less, about 18 months or less, about 20 months or less, about 22 months or less, about 25 months or less, or about 30 months or less, about 35 months or less, about 40 months or less, about 45 months or less, about 50 months or less, about 55 months or less, about 60 months or less, about 65 months or less, about 70 months or less, about 75 months or less, about 80 months or less, about 85 months or less, about 90 months or less, about 95 months or less, about 100 months or less, about 150 months or less, about 200 months or less, or about 250 months or less. In some embodiments, the method further comprises administering a differing amount of the anti-cancer agent. For example, subjects with relatively shorter overall survival can be administered higher amounts of the anti-cancer agent; subjects with relatively longer overall survival can be administered lower amounts of the anti-cancer agent.

In some embodiments, the anti-cancer agent can be selected from the group consisting of cordycepin, fenretinide, Zyclara, vemurafenib (Zelboraf®), dabrafenib (Tafinlar®), encorafenib (Braftovi®), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afnitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex®), irinotecan (Camptosar®), mitomycin, doxorubicin (Adriamycin), methotrexate, vinblastine (CMV), durvalumab (Imfinzi®), avelumab (Bavencio®), Erdafitinib (Balversa), dacarbazine (DTIC), epirubicin, temozolomide (Temodar®), gemcitabine (Gemzar®), trabectedin (Yondelis®), and Pazopanib (Votrient).

In some embodiments, the subject has or is suspected of having LGG. Accordingly, the method of any preceding aspect further comprises administering an anti-LGG agent to the subject based on the prediction of the subject as having a shorter overall survival, wherein the anti-LGG agent is selected from the group consisting of cordycepin and fenretinide. In some embodiments, the method further comprises administering a differing amount of the anti-LGG agent. For example, subjects with relatively shorter overall survival can be administered higher amounts of the anti-LGG agent; subjects with relatively longer overall survival can be administered lower amounts of the anti-LGG agent, wherein the anti-LGG agent is selected from the group consisting of cordycepin and fenretinide.

In some embodiments, the subject has or is suspected of having melanoma (e.g., skin cutaneous melanoma). Accordingly, the method of any preceding aspect further comprises administering an anti-melanoma agent to the subject based on the prediction of the subject as having a shorter overall survival, wherein the anti-melanoma agent is selected from the group consisting of imiquimod cream (Zyclara), a BRAF inhibitor (e.g., vemurafenib (Zelborafg), dabrafenib (Tafinlar®), or encorafenib (Braftovi®)), or a checkpoint inhibitor (e.g., pembrolizumab (Keytruda) or nivolumab (Opdivo)). In some embodiments, the method further comprises administering a differing amount of the anti-melanoma agent. For example, subjects with relatively shorter overall survival can be administered higher amounts of the anti-melanoma agent; subjects with relatively longer overall survival can be administered lower amounts of the anti-melanoma agent, wherein the anti-melanoma agent is selected from the group consisting of imiquimod cream (Zyclara), a BRAF inhibitor (e.g., vemurafenib (Zelboraf®), dabrafenib (Tafinlar®), or encorafenib (Braftovi®)), or a checkpoint inhibitor (e.g., pembrolizumab (Keytruda) or nivolumab (Opdivo)).

In some embodiments, the subject has or is suspected of having breast cancer. Accordingly, the method of any preceding aspect further comprises administering an anti-breast cancer agent to the subject based on the prediction of the subject as having a shorter overall survival, wherein the anti-breast cancer agent is selected from the group consisting of anthracyclines, Taxanes, 5-fluorouracil (5-FU), Cyclophosphamide (Cytoxan), Carboplatin (Paraplatin), Platinum agents (cisplatin, carboplatin), Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), and Atezolizumab (Tecentriq). In some embodiments, the method further comprises administering a differing amount of the anti-breast cancer agent. For example, subjects with relatively shorter overall survival can be administered higher amounts of the anti-breast cancer agent; subjects with relatively longer overall survival can be administered lower amounts of the anti-breast cancer agent, wherein the anti-breast cancer agent is selected from the group consisting of anthracyclines, Taxanes, 5-fluorouracil (5-FU), Cyclophosphamide (Cytoxan), Carboplatin (Paraplatin), Platinum agents (cisplatin, carboplatin), Vinorelbine (Navelbine), Capecitabine (Xeloda), Gemcitabine (Gemzar), Ixabepilone (Ixempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), and Atezolizumab (Tecentriq).

In some embodiments, the subject has or is suspected of having a non-small cell carcinoma (NSCLC) (e.g., lung squamous cell carcinoma or lung adenocarcinoma). Accordingly, the method of any preceding aspect further comprises administering an anti-non-small cell carcinoma (NSCLC) (e.g., lung squamous cell carcinoma or lung adenocarcinoma) agent to the subject based on the prediction of the subject as having a shorter overall survival, wherein the anti-non-small cell carcinoma (NSCLC) (e.g., lung squamous cell carcinoma or lung adenocarcinoma) agent is selected from the group consisting of Cisplatin, Paclitaxel (Taxol), Albumin-bound paclitaxel (nab-paclitaxel, Abraxane), Docetaxel (Taxotere), Gemcitabine (Gemzar), Etoposide (VP-16), Pemetrexed (Alimta), Bevacizumab (Avastin), Ramucirumab (Cyramza), Nivolumab (Opdivo) and pembrolizumab (Keytruda), Atezolizumab (Tecentriq), and Vinorelbine (Navelbine). In some embodiments, the method further comprises administering a differing amount of the anti-non-small cell carcinoma (NSCLC)(e.g., lung squamous cell carcinoma or lung adenocarcinoma) agent. For example, subjects with relatively shorter overall survival can be administered higher amounts of the anti-non-small cell carcinoma (NSCLC) (e.g., lung squamous cell carcinoma or lung adenocarcinoma) agent; subjects with relatively longer overall survival can be administered lower amounts of the anti-non-small cell carcinoma (NSCLC) (e.g., lung squamous cell carcinoma or lung adenocarcinoma) agent, wherein the anti-non-small cell carcinoma (NSCLC) (e.g., lung squamous cell carcinoma or lung adenocarcinoma) agent is selected from the group consisting of Cisplatin, Paclitaxel (Taxol), Albumin-bound paclitaxel (nab-paclitaxel, Abraxane), Docetaxel (Taxotere), Gemcitabine (Gemzar), Etoposide (VP-16), Pemetrexed (Alimta), Bevacizumab (Avastin), Ramucirumab (Cyramza), Nivolumab (Opdivo) and pembrolizumab (Keytruda), Atezolizumab (Tecentriq), and Vinorelbine (Navelbine).

In some embodiments, the subject has or is suspected of having esophageal cancer. Accordingly, the method of any preceding aspect further comprises administering an anti-esophageal cancer agent to the subject based on the prediction of the subject as having a shorter overall survival, wherein the anti-esophageal cancer agent is selected from the group consisting of Carboplatin and paclitaxel (Taxol); Cisplatin and 5-fluorouracil (5-FU); ECF: epirubicin (Ellence), cisplatin, and 5-FU (especially for gastroesophageal junction tumors); DCF: docetaxel (Taxotere), cisplatin, and 5-FU; Cisplatin with capecitabine (Xeloda); Oxaliplatin and either 5-FU or capecitabine; Irinotecan (Camptosar); Trifluridine and tipiracil (Lonsurf); Trastuzumab; Ramucirumab; and Pembrolizumab (Keytruda). In some embodiments, the method further comprises administering a differing amount of the anti-esophageal cancer agent. For example, subjects with relatively shorter overall survival can be administered higher amounts of the anti-esophageal cancer agent; subjects with relatively longer overall survival can be administered lower amounts of the anti-esophageal cancer agent, wherein the anti-esophageal cancer agent is selected from the group consisting of Carboplatin and paclitaxel (Taxol); Cisplatin and 5-fluorouracil (5-FU); ECF: epirubicin (Ellence), cisplatin, and 5-FU (especially for gastroesophageal junction tumors); DCF: docetaxel (Taxotere), cisplatin, and 5-FU; Cisplatin with capecitabine (Xeloda); Oxaliplatin and either 5-FU or capecitabine; Irinotecan (Camptosar); Trifluridine and tipiracil (Lonsurf); Trastuzumab; Ramucirumab; and Pembrolizumab (Keytruda).

In some embodiments, the subject has or is suspected of having cervical squamous cell carcinoma. Accordingly, the method of any preceding aspect further comprises administering an anti-cervical squamous cell carcinoma agent to the subject based on the prediction of the subject as having a shorter overall survival, wherein the anti-cervical squamous cell carcinoma agent is selected from the group consisting of Cisplatin plus 5-fluorouracil (5-FU), Carboplatin Paclitaxel (Taxol®), Topotecan Gemcitabine (Gemzar®), docetaxel (Taxotere®), ifosfamide (Ifex®), 5-fluorouracil (5-FU), irinotecan (Camptosar®), and mitomycin, Bevacizumab (Avastin®), and Pembrolizumab (Keytruda). In some embodiments, the method further comprises administering a differing amount of the anti-cervical squamous cell carcinoma agent. For example, subjects with relatively shorter overall survival can be administered higher amounts of the anti-cervical squamous cell carcinoma agent; subjects with relatively longer overall survival can be administered lower amounts of the anti-cervical squamous cell carcinoma agent, wherein the anti-cervical squamous cell carcinoma agent is selected from the group consisting of Cisplatin plus 5-fluorouracil (5-FU), Carboplatin Paclitaxel (Taxol®), Topotecan Gemcitabine (Gemzar®), docetaxel (Taxotere®), ifosfamide (Ifex®), 5-fluorouracil (5-FU), irinotecan (Camptosar®), and mitomycin, Bevacizumab (Avastin®), and Pembrolizumab (Keytruda).

In some embodiments, the subject has or is suspected of having bladder cancer (e.g., muscle invasive bladder cancer). Accordingly, the method of any preceding aspect further comprises administering an anti-bladder cancer agent to the subject based on the prediction of the subject as having a shorter overall survival, wherein the anti-bladder cancer agent is selected from the group consisting of Cisplatin plus fluorouracil (5-FU), Mitomycin with 5-FU, Gemcitabine and cisplatin, dose-dense methotrexate, vinblastine, doxoubicin (Adriamycin), cisplatin (DDMVAC), methotrexate, vinblastine (CMV), Gemcitabine and paclitaxel (GemTaxol), Atezolizumab (Tecentriq®), durvalumab (Imfinzi®), avelumab (Bavencio®), Nivolumab (Opdivo®), pembrolizumab (Keytruda®), and Erdafitinib (Balversa). In some embodiments, the method further comprises administering a differing amount of the anti-bladder cancer agent. For example, subjects with relatively shorter overall survival can be administered higher amounts of the anti-bladder cancer agent; subjects with relatively longer overall survival can be administered lower amounts of the anti-bladder cancer agent, wherein the anti-bladder cancer agent is selected from the group consisting of Cisplatin plus fluorouracil (5-FU), Mitomycin with 5-FU, Gemcitabine and cisplatin, dose-dense methotrexate, vinblastine, doxorubicin (Adriamycin), cisplatin (DDMVAC), methotrexate, vinblastine (CMV), Gemcitabine and paclitaxel (GemTaxol), Atezolizumab (Tecentriq®), durvalumab (Imfinzi®), avelumab (Bavencio®), Nivolumab (Opdivo®), pembrolizumab (Keytruda®), and Erdafitinib (Balversa).

In some embodiments, the subject has or is suspected of having soft tissue sarcoma. Accordingly, the method of any preceding aspect further comprises administering an anti-soft tissue sarcoma agent to the subject based on the prediction of the subject as having a shorter overall survival, wherein the anti-soft tissue sarcoma agent is selected from the group consisting of ifosfamide (Ifex®) and doxorubicin (Adriamycin®), dacarbazine (DTIC), epirubicin, temozolomide (Temodar®), docetaxel (Taxotere®), gemcitabine (Gemzar®), vinorelbine (Navelbine®), trabectedin (Yondelis®), eribulin (Halaven®), and Pazopanib (Votrient). In some embodiments, the method further comprises administering a differing amount of the anti-soft tissue sarcoma agent. For example, subjects with relatively shorter overall survival can be administered higher amounts of the anti-soft tissue sarcoma agent; subjects with relatively longer overall survival can be administered lower amounts of the anti-soft tissue sarcoma agent, wherein the anti-soft tissue sarcoma agent is selected from the group consisting of ifosfamide (Ifex®) and doxorubicin (Adriamycin®), dacarbazine (DTIC), epirubicin, temozolomide (Temodar®), docetaxel (Taxotere®), gemcitabine (Gemzar®), vinorelbine (Navelbine®), trabectedin (Yondelis®), eribulin (Halaven®), and Pazopanib (Votrient).

In some aspects, disclosed herein is a method for treating a cancer in a subject, comprising:
isolating a nucleic acid from a biological sample derived from the subject;
sequencing a first polynucleotide encoding a complementarity determining region (CDR) domain of an antigen-binding protein and a second polynucleotide encoding a protein associated with the cancer;
determining a complementarity score of the CDR domain and the protein associated with the cancer, comprising multiplying a net charge per residue (NCPR) of the CDR domain by a value of change in charge due to an amino acid substitution in the protein associated with the cancer and further by “−1”;
and administering to the subject a therapeutically effective amount of an anti-cancer agent if the complementarity score is lower in the biological sample derived from the subject compared to a reference control.

In some aspects, disclosed herein is a method for treating a cancer in a subject, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a complementarity score of the CDR domain and the protein associated with the cancer, comprising multiplying a Uversky hydropathy score of the CDR domain by a value of change in Uversky hydropathy score due to an amino acid substitution in the protein associated with the cancer;
and administering to the subject a therapeutically effective amount of an anti-cancer agent if the complementarity score is lower in the biological sample derived from the subject compared to a reference control.

In some aspects, disclosed herein is a method for treating a cancer in a subject, comprising.
isolating a nucleic acid from a biological sample derived from the subject;
sequencing a first polynucleotide encoding a complementarity determining region (CDR) domain of an antigen-binding protein and second polynucleotide encoding a protein associated with the cancer;
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer;
determining a mean z-score by averaging a first z-score of the first complementarity score and a second z-score of the second complementarity score, wherein the first z-score and the second z-score are relative to a reference database; and
administering to the subject a therapeutically effective amount of an anti-cancer agent if the mean z-score is lower in the biological sample derived from the subject compared to a reference control.

In some embodiments, the reference control is the complementarity score of a CDR domain and a protein associated with a cancer for the lowest 10%, 20%, 30%, 40%, or 50% of the complementarity score of a reference population of patient samples having the cancer. In some embodiments, the reference control is the complementarity score of a CDR domain and a protein associated with a cancer for the highest 10%, 20%, 30%, 40%, or 50% of the complementarity score of a reference population of patient samples having the cancer. Accordingly, the subject has a shorter overall survival if the complementarity score is lower in the biological sample derived from the subject compared to the reference control, and the subject has a longer overall survival if the complementarity score is higher in the biological sample derived from the subject compared to a reference control.

In some embodiments, the subject that is administered with an anti-cancer agent comprises the subject having an overall survival of about 1 month or less, about 2 months or less, about 4 months or less, about 6 months or less, about 8 months or less, about 10 months or less, about 12 months or less, about 14 months or less, about 16 months or less, about 18 months or less, about 20 months or less, about 22 months or less, about 25 months or less, or about 30 months or less, about 35 months or less, about 40 months or less, about 45 months or less, about 50 months or less, about 55 months or less, about 60 months or less, about 65 months or less, about 70 months or less, about 75 months or less, about 80 months or less, about 85 months or less, about 90 months or less, about 95 months or less, about 100 months or less, about 150 months or less, about 200 months or less, or about 250 months or less.

In some aspects, a complementarity score of a CDR domain and a protein associated with a cancer can also be determined by multiplying a net charge per residue (NCPR) of the CDR domain by a value of change in charge due to an amino acid substitution in the protein associated with the cancer. For example, an R->H mutation in a protein associated with a cancer (e.g., IGG) yields a −1.0 net change in charge at physiological pH, which if multiplied by a CDR3 domain NCPR of +0.5 yields a complementarity score of −0.5.

Accordingly, in some embodiments, a complementarity score that is less than 0 denotes the subject as having a longer overall survival. Accordingly, in some embodiments, the subject is administered with a therapeutically effective amount of an anti-cancer agent if the complementarity score is more than 0.

In some embodiments, the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some embodiments, wherein the protein associated with a cancer is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

In some embodiments, the anti-cancer agent is selected from the group consisting of cordycepin, fenretinide, Zyclara, vemurafenib (Zelboraf®), dabrafenib (Tafinlar®), encorafenib (Braftovi®), pembrolizumab (Keytruda), nivolumab (Opdivo), Anthracyclines, Taxanes, 5-fluorouracil (5-FU), Cyclophosphamide (Cytoxan), Carboplatin (Paraplatin), cisplatin, carboplatin, Vinorelbine (Navelbine), Capecitabine (Xeloda), Gemcitabine (Gemzar), Ixabepilone (lxempra), Eribulin (Halaven), Fulvestrant (Faslodex), Letrozole (Femara), Anastrozole (Arimidex), exemestane (Aromasin), Trastuzumab (Herceptin), Pertuzumab (Perjeta), Ado-trastuzumab emtansine, Lapatinib (Tykerb), Neratinib (Nerlynx), Everolimus (Afinitor), Olaparib (Lynparza), talazoparib (Talzenna), Alpelisib (Piqray), Atezolizumab (Tecentriq), Paclitaxel (Taxol), Albumin-bound paclitaxel (nab-paclitaxel, Abraxane), Docetaxel (Taxotere), Etoposide (VP-16), Pemetrexed (Alimta), Bevacizumab (Avastin), Ramucirumab (Cyramza), ifosfamide (Ifex®), irinotecan (Camptosar®), mitomycin, doxorubicin (Adriamycin), methotrexate, vinblastine (CMV), durvalumab (Imfinzi®), avelumab (Bavencio®), Erdafitinib (Balversa), dacarbazine (DTIC), epirubicin, temozolomide (Temodar®), gemcitabine (Gemzar®), trabectedin (Yondelis®), and Pazopanib (Votrient).

As would be apparent, the sequencing may be done using a next generation sequencing platform, e.g., Illumina's reversible terminator method, Roche's pyrosequencing method, Life Technologies' sequencing by ligation (the SOLiD platform) or Life Technologies' Ion Torrent platform, etc. Examples of such methods are described in the following references: Margulies et al (Nature 2005 437: 376-80); Ronaghi et al (Analytical Biochemistry 1996 242: 84-9); Shendure (Science 2005 309: 1728); Imelfort et al (Brief Bioinform. 2009 10:609-18); Fox et al (Methods Mol Biol. 2009; 553:79-108); Appleby et al (Methods Mol Biol. 2009: 513:19-39) and Morozova (Genomics. 2008 92:255-64), which are incorporated by reference for the general descriptions of the methods and the particular steps of the methods, including all starting products, reagents, and final products for each of the steps. In other embodiments, the sequencing may be done using nanopore sequencing (e.g. as described in Soni et al Clin Chem 53: 1996-2001 2007, or as described by Oxford Nanopore Technologies).

### Methods of Screening

In some aspects, disclosed herein is a method for screening an antigen-binding protein for treating a cancer, comprising:
obtaining a first sequence of a CDR domain of the antigen-binding protein and a second sequence of a protein associated with the cancer;
determining a complementarity score of the CDR domain and the protein associated with the cancer; and
determining the antigen-binding protein for treating the cancer if the complementarity score thereof is higher compared to a reference control.

In some aspects, disclosed herein is a method for screening an antigen for a cancer vaccine, comprising:
obtaining a first sequence of a CDR domain of the antigen-binding protein derived from a subject and a second sequence of the antigen for the cancer vaccine;
determining a complementarity score of the CDR domain and the antigen; and
determining the antigen as the antigen for the cancer vaccine if the complementarity score thereof is higher than a reference control.

In some embodiments, the first sequence and the second sequence are nucleotide sequences. In some embodiments, the first sequence and the second sequence are amino acid sequences.

The antigen-binding protein used herein can be, for example, a TCR, a BCR, or an antibody, or a functional fragment thereof. In some embodiments, the antigen-binding protein is a TCR or a functional fragment thereof. In some embodiments, the antigen-binding protein is TCR alpha chain or a functional fragment thereof. In some embodiments, the antigen-binding protein is TCR beta chain or a functional fragment thereof. In some embodiments, the antigen-binding protein is TCRγ or a functional fragment thereof. In some embodiments, the antigen-binding protein is TCRδ or a functional fragment thereof.

In some embodiments, the antigen-binding protein is a BCR or an antibody or a functional fragment thereof. In some embodiments, the antigen-binding protein is a BCR or a functional fragment thereof. In some embodiments, the antigen-binding protein is an antibody or a functional fragment thereof. As noted above, the isotype of an antibody or a BCR is can be IgM, IgD, IgG, IgA, and IgE. Accordingly, in some embodiments, the antigen-binding protein is an IgG antibody or a functional fragment thereof. In some embodiments, the antigen-binding protein is an IgG BCR or a functional fragment thereof. In some embodiments, the antigen-binding protein is a light chain of an IgG antibody or a functional fragment thereof. In some embodiments, the antigen-binding protein is a heavy chain of an IgG antibody or a functional fragment thereof. In some embodiment, antigen-binding protein is a light chain of IgG BCR or a functional fragment thereof. In some embodiments, the antigen-binding protein is a heavy chain of IgG BCR or a functional fragment thereof.

In some embodiments, the CDR is a CDR1 of a light chain of an antibody. In some embodiments, the CDR is a CDR2 of a light chain of an antibody. In some embodiments, the CDR is a CDR3 of a light chain of an antibody. In some embodiments, the CDR is a CDR1 of a heavy chain of an antibody. In some embodiments, the CDR is a CDR2 of a heavy chain of an antibody. In some embodiments, the CDR is a CDR3 of a heavy chain of an antibody. In some embodiments the antibody of any preceding aspects is selected from the group consisting of IgM, IgD, IgG, IgA, and IgE.

In some embodiments, the CDR is a CDR1 of a light chain of a BCR. In some embodiments, the CDR is a CDR2 of a light chain of a BCR. In some embodiments, the CDR is a CDR3 of a light chain of a BCR. In some embodiments, the CDR is a CDR1 of a heavy chain of a BCR. In some embodiments, the CDR is a CDR2 of a heavy chain of a BCR. In some embodiments, the CDR is a CDR3 of a heavy chain of a BCR. In some embodiments the BCR of any preceding aspects is selected from the group consisting of IgM, IgD, IgG, IgA, and IgE.

In some embodiments, the CDR is a CDR1 of an alpha chain of a TCR. In some embodiments, the CDR is a CDR2 of an alpha chain of a TCR. In some embodiments, the CDR is a CDR3 of an alpha chain of a TCR. In some embodiments, the CDR is a CDR1 of a beta chain of a TCR. In some embodiments, the CDR is a CDR2 of a beta chain of a TCR. In some embodiments, the CDR is a CDR3 of a beta chain of a TCR.

In some embodiments, disclosed herein is a method for predicting the CDR is a CDR of a γ chain of a γδTCR. In some embodiments, the CDR is a CDR of a δ chain of a γδTCR.

In some embodiments, the complementarity score of the CDR domain of the antigen-binding protein and the protein associated with the cancer is determined by multiplying a net charge per residue (NCPR) of the CDR domain by a value of change in charge due to an amino acid substitution in the protein associated with the cancer and further by “−1”.

In some embodiments, the complementarity score can be determined by multiplying a NCPR of a CDR domain by a value of change in charge due to an amino acid substitution in a protein associated with a cancer. In some embodiments, the complementarity score can be determined by multiplying one or more NCPRs of one or more CDR domains by one or more values of change in charge due to one or more amino acid substitutions in one or more proteins associated with a cancer.

In some embodiments, the complementarity score of the CDR domain of the antigen-binding protein and the protein associated with the cancer is determined by multiplying a Uversky hydropathy score of the CDR domain by a value of change in Uversky hydropathy score due to an amino acid substitution in the protein associated with the cancer.

In some embodiments, the complementarity score of the CDR domain of the antigen-binding protein and the protein associated with the cancer is determined by
determining a first complementarity score of the CDR domain and the protein associated with the cancer, comprising multiplying a net charge per residue (NCPR) of the CDR domain by a value of change in charge due to an amino acid substitution in the protein associated with the cancer and further by “−1”;
determining a second complementarity score of the CDR domain and the protein associated with the cancer, comprising multiplying a Uversky hydropathy score of the CDR domain by a value of change in Uversky hydropathy score due to an amino acid substitution in the protein associated with the cancer; and
determining a mean z-score by averaging a first z-score of the first complementarity score and a second z-score of the second complementarity score, wherein the first z-score and the second z-score are relative to a reference database; and
wherein the complementarity score is equal to the mean z-score.

In some embodiments, the reference control is the complementarity score for the lowest quintile (20%) of the complementarity scores of a reference population having the cancer. In some embodiments, the reference control is the complementarity score for the highest quintile (20%) of the complementarity scores of a reference population having the cancer.

In some embodiments, the reference control is the complementarity score for the lowest 10%, 20%, 30%, 40%, or 50% of the complementarity scores of a reference population having the cancer. In some embodiments, the reference control is the complementarity score for the highest 10%, 20%, 30%, 40%, or 50% of the complementarity scores of a reference population having the cancer.

In some embodiments, the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

In some embodiments, the protein associated with a cancer (or the antigen for cancer vaccine) is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF), Tumor protein p53 (TP53), ATP-dependent helicase ATRX (ATRX), Neuroblastoma RAS viral oncogene homolog (NRAS), and Retinoblastoma protein (RB1), or a functional fragment thereof.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. T-Cell Receptor (TCR), Mutant Peptide Complementarity Scoring for Identification of Effective TCR-Mutant Peptide Pairs for Therapies and Prognoses

Many studies have supported the conclusion that bladder cancer patients with a T-cell infiltrate have a better prognosis, although bladder cancer recurrence can be associated with an over-abundance of regulatory T-cells. In a variety of other cancers, there have been similar findings, with melanoma generally representing the greatest advances with regard to identification of naturally occurring, specific TCRs against specific cancer antigens; and CD19-positive lymphomas representing the greatest advances with common-antigen directed T-cell killing.

These successes have raised the issue of how to identify, and exploit, naturally occurring T-cells that effectively recognize cancer antigens that occur in multiple patients. Such processes of identification allow for a more consistent evaluation of patients, in the sense that it is highly unlikely that a single TCR amino acid (AA) sequence, evidenced to have effectiveness in one patient, will have the same effectiveness in the next patient. This problem is due to two issues. First, the TCR V and J usage, and possibly the CDR3, are restricted to HLA types, and recent work has indicated that only certain TCR V and J usage, HLA allele combinations are associated with the highest survival rates. Second, effective cancer antigens, and neo-antigens, vary from patient to patient. To a certain extent, these problems are evaded by the preparation of patient tumor infiltrating lymphocytes (TILs) ex vivo, but this approach on a patient to patient basis has mixed results, with many patients expiring prior to sufficient ex-vivo replication of TILs.

Despite the above indicated challenges to a “pre-identification” of an effective TCR sequence, patients can have HLA types in common and cancer antigens in common, supporting a careful consideration of how these commonalities can be exploited. In the case of HLA types, over many years of effort, there have been a few examples of HLA types associated with the development of certain cancers. And as noted, it is possible to detect at least preliminary indications of V and J usage associated with a distinct outcome, although there is to date no empirical evidence supporting that work. Furthermore, the statistical requirements of validating and re-validating the association of specific V and J usage, HLA allele combinations with survival rates, especially given the impractically of establishing clinical trials with a patient cohort make-up consisting of a specific HLA allele distribution, makes it likely that progress in HLA related prognosis or therapy design will be slow. In addition, the antigenic variation, from tumor to tumor, may reduce the predictive value of particular V and Jusage, HLA allele combinations. In particular, in the previous studies associating V and J usage, HLA allele combinations with survival rates, no consistent CDR3 feature(s) was apparent.

Although specific AA sequences of the antigen-binding CDR3 are not repeated from patient to patient, patients with the same driver or cancer specific mutations, can reveal CDR3 regions with common chemical features, presumably reflecting common cancer antigens. Addressing this prospect is facilitated by the relatively convenient availability of very large numbers of immune receptor recombination reads from tumor exome files, where extensive studies have firmly supported the conclusion that such immune receptor recombination reads represent TILs. Particularly, in the case of bladder cancer, results indicated the usefulness of bioinformatics approaches to the assessment of CDR3 chemistry in advancing the usefulness of TCR data for patient prognosis and therapy design.

### Methods

Recovery of immune receptor V(D)J recombination reads. The genomic data commons (GDC) web portal (portal.gdc.cancer.gov/) was queried for bladder cancer (BLCA) whole exome sequence (WXS) files. Four hundred eighteen primary tumor WXS and 394 blood WXS files were downloaded to USF research computing using the GDC data transfer tool (version 1.3). V(D)J recombination read recovery was performed in two stages: the first stage used a collection of scripts similar to previous publications, and the second stage performed pairwise alignment of candidate reads to known V and J sequences. Known V and J sequences were obtained from The International Immunogenetics Information System (www.imgt.org/). The quantitative parameters for the pairwise alignment were: (i) nucleotide match, +5; (ii) mismatch, −10; (iii) opening gap, −10; and (iv) extending gap, −10. The threshold for a V or J gene segment match was a score greater than or equal to 65. The required parameters for V(D)J recombination read identification were reads containing both a V and J region, a productive in-frame junction with no stop codons, a match length of at least 20 nucleotides for each gene segment, and at least a 90% nucleotide match fidelity.

Assessment of physico-chemical features of CDR3 regions. V(D)J recombination read CDR3 domains were identified and translated using an original script. Isoelectric point, fraction of residues with a positive charge, and net charge per residue (NCPR) were calculated with the localCIDER python package (pappulab.github.io/localCIDER/).

Assessment of survival associations with IRA CDR3 chemical features. Survival correlations with TRA CDR3 physico-chemical features were performed by separating barcodes into top and bottom halves or quintiles based on each individual chemical feature, then performing a Kaplan-Meyer (KM) survival analysis comparing the top and bottom groups, using GraphPad Prism software (version 7), which also outputted figures.

Assessment of survival associations for gene mutants and non-mutants overlapping TRA CDR3s with specific chemical features. Open-access Mutect gene mutation information was obtained from the GDC web portal (portal.gdc.cancer.gov/). Using an original script, barcodes were separated into mutant and non-mutant groups. Both groups were further separated by highest and lowest halves based on the quantification of particular physico-chemical properties. KM survival analyses were performed comparing highest versus lowest physico-chemical property barcode group, within a given gene mutation, barcode set; and by comparing highest vs lowest physicochemical property barcode groups within a barcode set that lack mutations in the corresponding gene. These two KM analyses were performed for every physicochemical property, for every gene for which mutation information was available, for both overall survival (OS) and disease-free survival (DFS). Genes for which an OS or DFS distinction based on a physico-chemical property was observed, among the patients also having a particular mutated gene, but not among the patients lacking a mutation in that gene, were further considered as indicated in Results.

### Results

To assess correlations between TCR CDR3 domain physico-chemical properties and survival, V(D)J recombination read recovery was first performed on 418 TCGA BLCA primary tumor WXS files. CDR3 regions of the TRA recombination reads were translated into an AA sequence and analyzed first for isoelectric point.

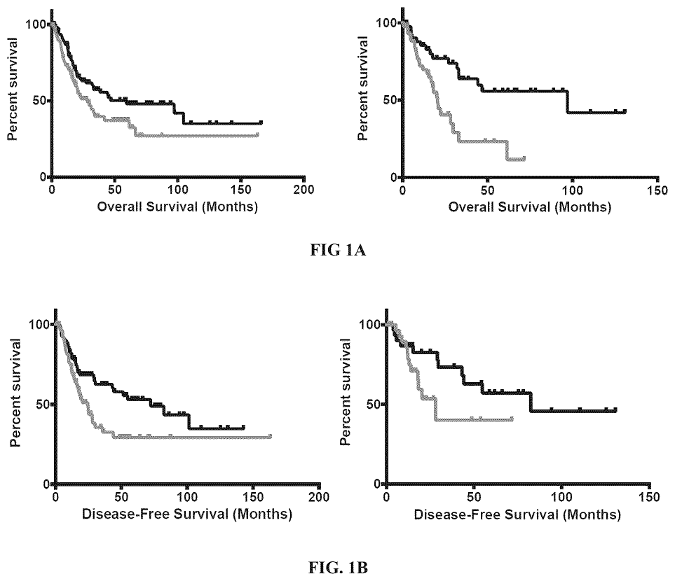

Overall-survival (OS) and disease-free survival (DFS) represented by barcodes representing the highest half of TRA CDR3 isoelectric points were compared to the barcodes representing the lowest half of isoelectric points using Kaplan-Meyer (KM) survival analysis (Table 1). Note in particular that the isoelectric points, assessed as in the Methods, of the top and bottom fiftieth percentiles, were significantly different (Table 1). Barcodes representing the lowest half of TRA CDR3 isoelectric points represented significantly increased OS (n=112, p=0.027) and DFS (n=85, p=0.035) compared to barcodes representing the highest half of isoelectric points, for the TRA CDR3s (FIG. 1). Similar analyses of the CDR3 domains represented by the TRB, TRG, TRD, IGH, IGK, and IGL recombination reads recovered from the bladder tumor tissue samples did not indicate any statistically significant survival associations. Also, no chemical features of the CDR3 domains represented by the TRA recombination reads recovered from bladder cancer patient blood WXS files correlated with survival distinctions.

TABLE 1
KM analyses for barcode groups of the highest versus lowest, TRA CDR3 isoelectric points, as
determined by recovery of TRA recombination reads from TCGA BLCA WXS files.

| Tissue source | | Lowest 50% | Highest 50% | Isoelectric | | |
|---|---|---|---|---|---|---|
| of TRA | | average | average | point | | |
| recombination | | isoelectric | isoelectric | p-value | Survival Log | Number of |
| reads | Survival | point | point | distinction | rank p-value | barcodes |
| Primary Tumor | Overall | 5.80 | 8.16 | <0.0001 | 0.039 | 224 |
| Primary Tumor | Disease-Free | 5.84 | 8.09 | <0.0001 | 0.020 | 172 |
| Blood | Overall | 6.12 | 8.18 | <0.0001 | 0.624 | 326 |
| Blood | Disease-Free | 6.12 | 8.14 | <0.0001 | 0.347 | 265 |

Note:
The lower 50% group represented the better survival rate in the case of primary tumor.

To assess relationships between tumor-resident TRA CDR3 domain physicochemical properties and mutant proteins in the tumor, Mutect mutation information obtained from the GDC was used. For each gene for which mutation data was available, barcodes were separated into mutated-gene and nonmutated-gene groups. These two groups were subdivided by highest and lowest, fiftieth percentile, TRA CDR3 isoelectric points. Two KM survival analyses were then performed. The first KM analysis compared the survival rates represented by the barcodes with gene mutations and the highest half of isoelectric point values for the TRA CDR3s, versus barcodes with mutations (in the same gene) and the lowest half of the isoelectric point values for the TRA CDR3s. A second KM analysis compared barcodes representing the lack of mutations for the same gene and with the highest half of isoelectric point values for the TRA CDR3s, versus barcodes lacking mutations but with the lowest half of TRA CDR3 isoelectric values.

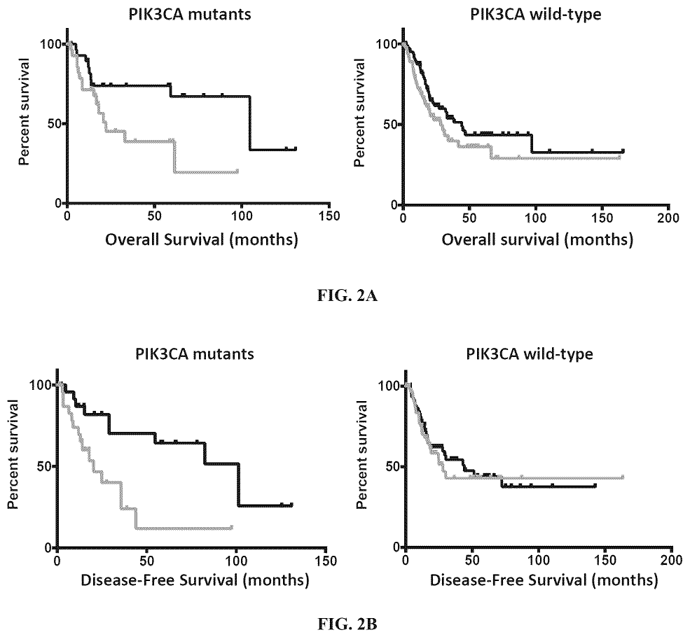

Only several genes, out of a total of 14,256 mutated genes in the TCGA BLCA dataset, were identified whereby a low CDR3 isoelectric point value correlated with a significantly increased OS and DFS for the mutated gene barcode set but not for the non-mutated gene barcode set (Table 2). Interestingly, in 7 out of the 8 genes identified, the average change in isoelectric point, due to the mutant amino acid substitution, was positive, i.e., there was an apparent isoelectric point change in the mutant protein that was complementary to the TRA CDR3 isoelectric point group that represented the higher survival rate. The mutated genes that encoded the greatest (complementary) change in isoelectric point, PIK3CA and ITPR2 (FIG. 2) are commonly mutated in bladder cancer (PIK3CA, 54 samples out of 227 samples with TRA read recovery from the BLCA WXS files; ITPR2, 26 out of 227).

TABLE 2
KM analyses for highest versus lowest isoelectric point groups among barcodes with gene mutations.

| | | | | | | Average |
|---|---|---|---|---|---|---|
| | | | | | | Isoelectric |
| | | | | | | point change |
| | Mutants | Non-Mutants | Mutants | Non-Mutants | | due to the |
| | p-value | p-value | p-value | p-value | | mutant |
| | (overall | (overall | (disease-free | (disease-free | | amino acid |
| Gene | survival) | survival) | survival) | survival) | n | substitution |
| PIK3CA | 0.055 | 0.095 | 0.009 | 0.642 | 54 | +2.76 |
| ITPR2 | 0.007 | 0.196 | 0.0007 | 0.181 | 26 | +1.26 |
| LAMA3 | 0.010 | 0.171 | 0.058 | 0.091 | 24 | +1.08 |
| NCOR1 | 0.030 | 0.165 | 0.007 | 0.088 | 27 | +0.74 |
| ZFHX4 | 0.005 | 0.181 | 0.002 | 0.064 | 27 | +0.21 |
| NEB | 0.012 | 0.188 | 0.017 | 0.091 | 39 | +0.17 |
| MDN1 | 0.046 | 0.160 | 0.025 | 0.124 | 26 | +0.12 |
| AHNAK2 | 0.050 | 0.216 | 0.015 | 0.100 | 42 | −0.46 |

Note:
Only genes representing cases where the gene mutated set, in comparison to the gene wild-type set, showed a statistically significant survival distinction, based on isoelectric point values, are listed in this table.

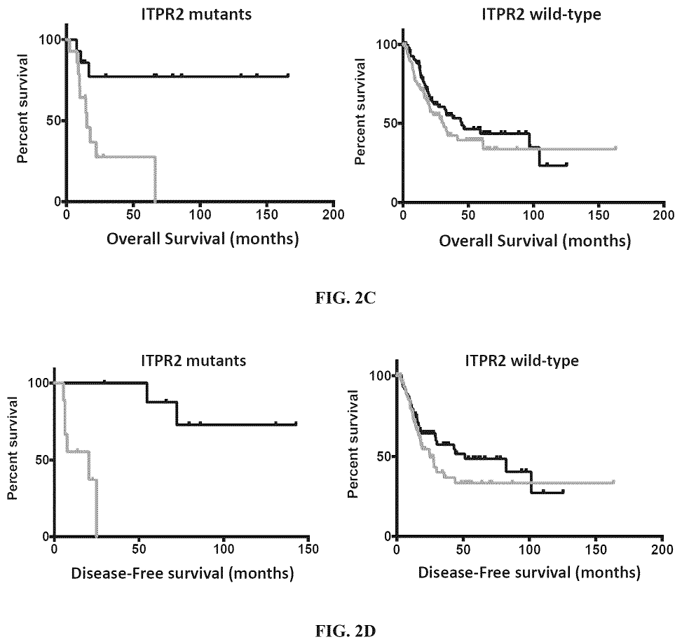

To assess whether other physico-chemical properties of the TRA CDR3s, recovered from the BLCA WXS files, were correlated with survival rates, similar analyses were performed by dividing barcodes into top and bottom fiftieth percentiles by aromaticity, hydropathy, molecular weight, and charge. It was found that lower net charge per residue (NCPR) (n=112, p=0.033), and fewer fraction of positive residues in the TRA CDR3 regions (n=112, p=0.038) also correlated with better OS (FIG. 3).

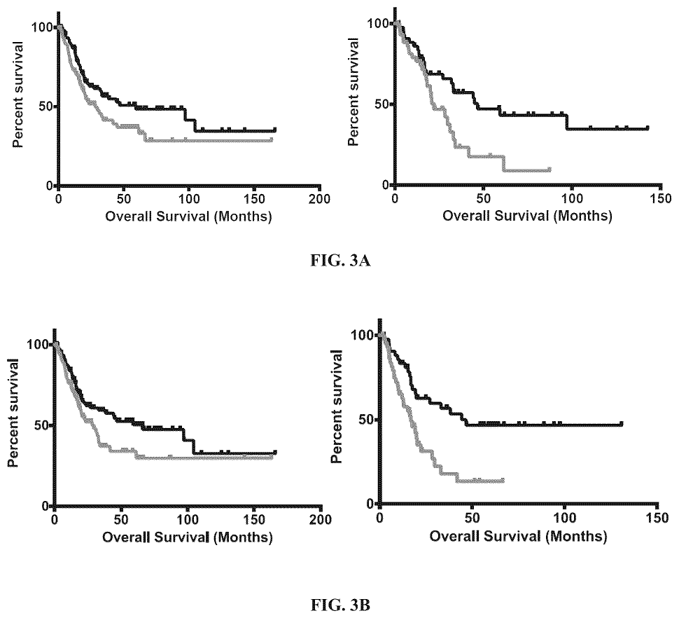

To determine whether the TRA CDR3 isoelectric point values, NCPR, or fraction of positive residues correlated with survival in other cancers, the above analyses were repeated on all TCGA cancer datasets. It was found that low isoelectric point values and low NCPR were correlated with increased OS (n=32, p=0.041) and DFS (n=24, p=0.0043) in esophageal cancer (ESCA) (FIG. 4A). Moreover, the two ESCA barcode sets represented by these isoelectric points and NCPR survival distinctions, respectively, overlapped exactly. The fraction of positive residues was associated with low OS in stomach adenoma (STAD) (n=78, p=0.0154) (FIG. 4B), and better OS in ovarian cancer (OV) (n=62, p=0.0147) (FIG. 4C).

Results of the above analysis are consistent with the idea that TILs with CDR3s with particular chemical features can be grouped to predict cancer survival rates. These results also show that the common chemical features of the longer surviving BLCA patients can represent specific interactions with common antigens. For example, of the 14,256 BLCA mutants analyzed, only 8 reflected a survival distinction based on a distinct, complementary CDR3 chemical feature, namely the TRA CDR3, low isoelectric point group. In addition, 7 out of these 8 reflected a complementary isoelectric point. Thus, the approach of identifying CDR3 chemical features associated with survival can identify tumor antigens common to more than one patient. Such antigens can ultimately be useful as vaccines or ways to increase ex-vivo stimulation of therapeutic TILs. Given that common CDR3s should identify common antigens, such antigens represent driver mutants, i.e., mutants appearing in numerous patients beyond what is expected by random chance.

### Example 2. Soft Tissue Sarcoma Survival Rates Correlate with t-Cell Receptor and Mutant Amino Acid Chemical Complementarities

The importance of T-Cell Receptor α (TCR-α) and the chemical properties of its complementarity determining region-3 (CDR3) domain have been indicated for distinguishing high and low survival patients in bladder cancer. A similar approach is investigated for soft tissue sarcoma (STS). Finding a connection between STS survival and the electrostatic complementarity of a patient's tumor antigens and their TCR-α CDR3 domain chemistry provides the use of individualized neoepitope vaccines in STS patients.

### Methods

Immune receptor V(D)J recombination reads were recovered from the whole exome sequence files, from the genomic data commons (GDC), representing 156 STS patients. The CDR3 domains of these V(D)J recombination reads were identified and translated. The net charge per residue (NCPR) was calculated for each CDR3. Mutect files (mutation data) were obtained from the GDC. The difference in the electrostatic charge caused by mutant amino acids (AA) was determined for each sample. The average product of the TCR-α CDR3 NCPR and mutant AA charge difference was calculated for each patient and represented the patient's complementarity score. Survival, clinical, and RNASeq data for 265 STS patients were obtained from cBioPortal (TCGA, Provisional). Kaplan-Meier curves were used for survival analysis. Pearson's correlation analysis and independent T tests were performed between complementarity scores and clinical characteristics, as well as for assessing RNASeq values.

### Results

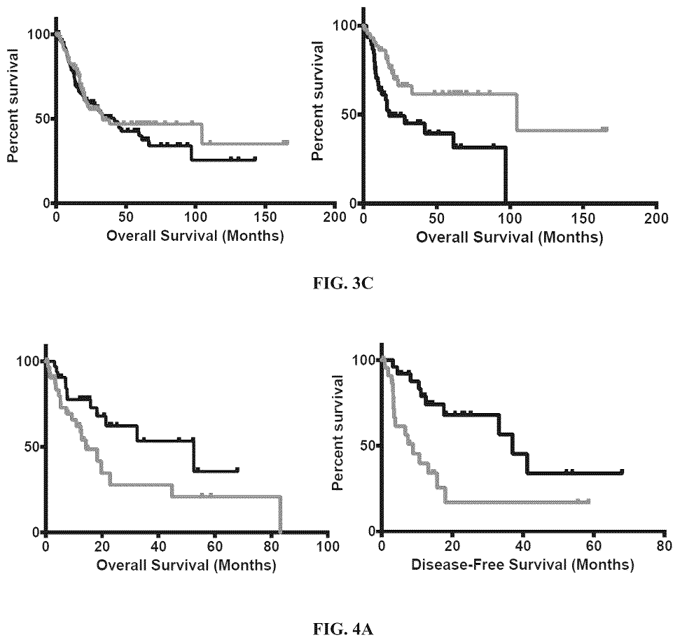

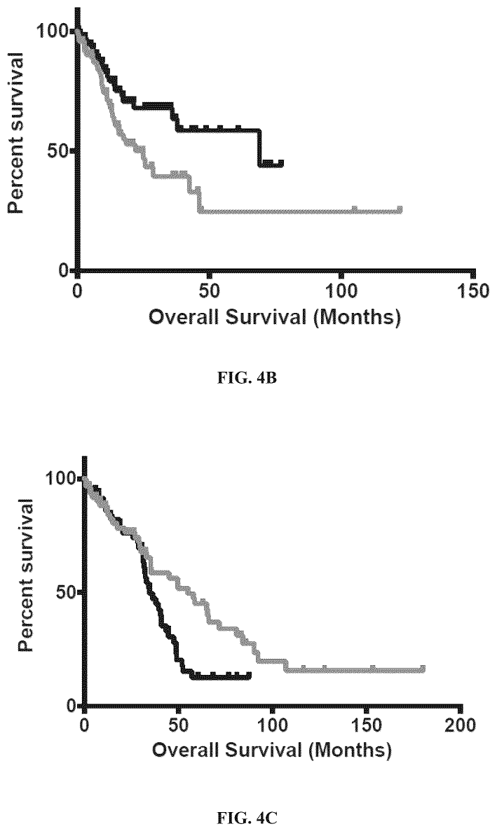

Improved overall survival (OS) was significantly correlated with complementarity between TCR-α CDR3 domains and electrostatic charge changes due to mutant AA (n=19). In contrast, lower OS was observed for patients lacking this complementarity (n=−34, p=0.051), for all other patients with TCR-α recoveries (n=118, p=0.016); and for all patients without immune receptor recoveries (n=105, p=0.012) (FIGS. 5A-5C). When comparing pro-apoptotic gene expression using RNASeq values, patients with CDR3-mutant AA electrostatic charge complementarity had significantly increased GZMA and GZMB expression in their tumors compared to all other patients with TCR-α recombination read recoveries (p=0.011 and 0.004, respectively) (FIGS. 6A and 6B). Survival analyses of the TCGA database showed that age at diagnosis, primary pathologic length, multifocal disease, fraction of genome altered, and surgical margin all correlated with OS in a univariate analysis. No correlation was found between these clinical characteristics and complementarity score. Patients with complementarity were distributed across STS subtypes. In other words, (CDR3-mutant AA complementarity was an independent survival marker.

STS patients with complementarity between TCR-α CDR3 domains and tumor antigens show improved OS, as well as increased expression of pro-apoptotic genes in their tumors, compared to patients who did not have this complementarity. The independent survival advantage found in this study can be due to tumor infiltrating lymphocytes having increased ability to recognize tumors by the physiochemical properties of their TCR-α polypeptides and provides support for the presence of immunogenic potential in STS setting. These findings are clinically relevant, as achieving electrostatic complementarity to tumor antigens is an important parameter for developing individualized neoepitope vaccines for patients with STS.

### Example 3. Electrostatic Complementarity of T-Cell Receptor-Alpha CDR3 Domains and Mutant Amino Acids is Associated with Better Survival Rates for Soft Tissue Sarcomas

Like many other cancer types, soft tissue sarcomas are being examined for the basic immune response characteristics of tumor infiltrating lymphocytes, for examples, lymphocyte clonality and immune checkpoint protein expression. Also, in vitro work, indicating molecular specifics of the immune targeting of soft tissue sarcoma cells, has progressed. However, there has been less progress with soft tissue sarcoma immunogenomics, particularly based on large sample sizes, in contrast to melanoma and other solid cancers.

Cancer immunogenomics has been approached in a number of ways. In early work, the approach was specific to isolated tumors. For example, T cell receptor (TCR) sequences facilitating the killing of a single tumor, based on specific tumor antigens, have been identified. More recently, bulk analyses of tumor infiltrating lymphocytes have provided a more global or “landscape” view of cancer immunogenomics. Recent cancer immunogenomics has basically represented one of two approaches: (a) immune repertoire sequencing; and (b) mining of exome (WXS) and RNASeq files for immune receptor recombination reads swept up in the production of those files, i.e., files primarily intended for mutation and expression analyses, respectively. While the immune repertoire approach, based on PCR-amplification of all, or almost all TCR recombinations present in a tumor sample, represents a clear opportunity for comprehensiveness, this approach is limited in terms of the number of samples that can be assessed at any one time, essentially due to cost.

Mining of WXS files is less comprehensive, in terms of any one sample, but such mining provides an opportunity to assess a very large collection of samples at one time; and to recover the recombinations represented by all seven adaptive immune receptor genes, at one time. Moreover, the immune receptor recombination read mining of WXS files has been substantially benchmarked, in the sense that the immune receptor recombination reads recovered from the WXS files represent tumor infiltrating lymphocytes (TILs). As just one of many examples, a recent report indicated that pancreatic cancer patients with intratumor bacteria had a worse outcome, while an essentially simultaneous, completely independent report indicated that B-cell receptor recombination reads (in the case of a Th2, bacterially initiated immune response), represented a significantly worse, pancreatic cancer outcome. More recent, WXS-immune receptor mining approaches have established links between TCR V or J usage, HLA allele occurrence and survival outcomes, and links between TCR CDR3 physical chemical features and survival outcomes, including chemical features that represent CDR3-mutant AA chemical complementarity. The immune repertoire approach has also provided a link between specific CDR3 physical chemical features and tumor infiltrating lymphocytes.

Thus, the study shown herein sought to extend the WXS, immune receptor mining to soft tissue sarcoma, with results indicating that soft tissue sarcoma patients with electrostatic complementarity between TCR-α CDR3-mutant AAs have improved overall survival, compared to patients who had non-complementary TRA recombination read recoveries from their tumors. Furthermore, patients with electrostatic complementarity between CDR3 and the most frequently mutated genes in the TCGA-SARC database: TP53, ATRX, and RB1, have an improved overall survival compared to patients who specifically do not have complementarity to mutant AAs in these three genes.

### Abbreviations

CDR3, complementarity domain region-3; GDC, genomic data commons; PCR, polymerase chain reaction; SARC, soft tissue sarcoma set from TCGA; STS, soft tissue sarcomas; TCGA, the cancer genome atlas; TCR, T-cell receptor; TIL, tumor infiltrating lymphocyte; TRA, T-cell receptor alpha gene; WXS, whole exome file.

### Methods

Recovery of IRA recombination reads. Whole exome sequence (WXS) files for TCGA-SARC case IDs representing 261 soft tissue sarcoma (STS) patients were recovered from the genomic data commons (GDC) (portal.gdc.cancer.gov/repository) per National Center for Biotechnology Information's Database of Genotypes and Phenotypes approved project number 6300. Immune receptor V(D)J recombination reads were recovered using previously published and extensively benchmarked processes, with read validations based on known V and J sequences obtained from The International Immunogenetics Information System (imgt.org/). The complementarity-determining region 3 (CDR3) domains of the V(D)J recombination reads were then identified and translated. Both processes were performed using a previously described algorithm, and the original script is at Github.com (github.com/bchobrut-USF/lgg_idh1). The net charge per residue (NCPR) was calculated for each CDR3 using the IocaICIDER python package (pappulab.github.io/localCIDER/).

TCGA-SARC mutation data and the calculation of complementarity scores (CS). Mutation data from Mutect files were obtained from the GDC. The difference in the electrostatic charge caused by mutant amino acids (AA) was determined for each sample. Specifically, glutamate and aspartate, mutant and wild-type AA were assigned −1.0; arginine, lysine, and histidine were assigned+1.0; all other AA were assigned zero. Each wild-type AA was then subtracted from the corresponding mutant AA. In cases where multiple AAs were altered, such as frameshifts, the net charge was calculated for each AA sequence, and a simple assignment of either −1.0 or +1.0 was given for each sequence with a negative or positive net charge, respectively; each wild-type AA was then subtracted from the corresponding mutant AA. The product of each patient's average TCR-α CDR3 NCPR and average mutant AA charge difference (i.e., an entire mutanome charge difference value) was calculated and represented the patient's CS. Note, in this type of calculation, the negative product (representing opposite electrostatic charge values) represented complementarity, for the subsequent analyses in this report, i.e., a negative value product represented high complementarity. Low complementarity, or lack of complementarity, was defined as a zero product or a positive value product.

Clinical correlates and RNASeq data used in this report. Survival, clinical, and RNASeq data for SARC case IDs representing 261 STS WXS files were obtained from cBioPortal (TCGA, Provisional). Kaplan-Meier (KM) curves were generated in GraphPad Prism and used for survival analysis. Pearson's correlation analysis and independent t tests were performed between complementarity scores and clinical characteristics in SPSS. RNASeq values were assessed using independent t-tests in Excel.

### Results

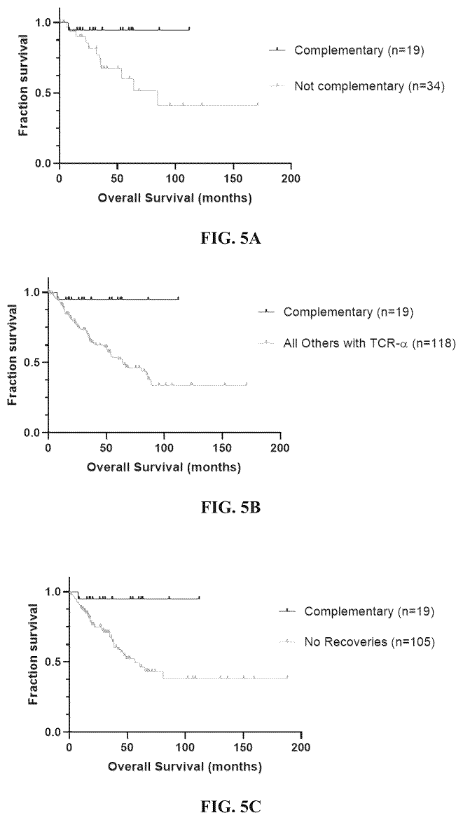

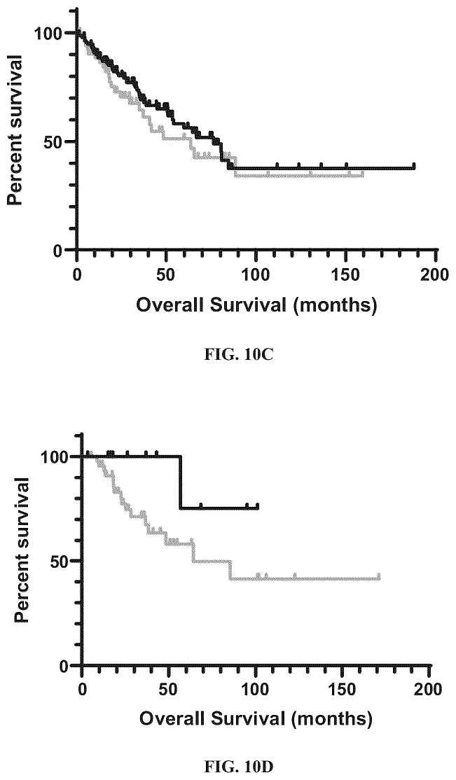

To determine whether T-cell receptor read recombinations in tumors were associated with distinct survival outcomes for the TCGA-SARC dataset, V(D)J recombination read recovery from WXS files was performed for 265 SARC primary and metastatic tumor samples. Kaplan-Meier (KM) survival analysis was used to compare the overall survival (OS) of SARC case IDs representing patients with TRA recombination read recoveries versus all remaining SARC case IDs. Results indicated that TRA recombination read recoveries were associated with a significantly improved OS rate, compared to lack of any TRA recombination read recovery from the tumor WXS files (Table 3; FIG. 12). To substantiate and further support the survival distinction, RNASeq values of immune markers and pro-apoptotic genes were obtained for the two groups representing either TRA recombination read recovery, or not, respectively. The RNASeq values of lymphocyte biomarkers CD4, CD8, and IFNG (Table 3), as well as the RNASeq values of pro-apoptotic genes CASP4, CASP5, GZMA, and GZMB (FIG. 7), were significantly increased in the tumor samples with TRA recombination read recoveries, compared to all remaining SARC samples. These results were thus consistent with the OS rate distinction between the two groups of patients.

TABLE 3
Initial characterization of TCGA-
SARC samples for immune biomarkers

| | TRA | | |
|---|---|---|---|
| | recombination | | |
| | read recovery | All Others | |
| | (n = 57) | (n = 204) | p-value |
| Median Survival | Undefined | 60.61 | 0.0482 |
| (months) | | | |
| CD4 RNASeq value | 4033.71 | 2106.64 | 7.04E−05 |
| CD8 RNASeq value | 952.96 | 220.45 | 0.000703 |
| IFNG RNASeq value | 27.53 | 7.29 | 0.002834 |

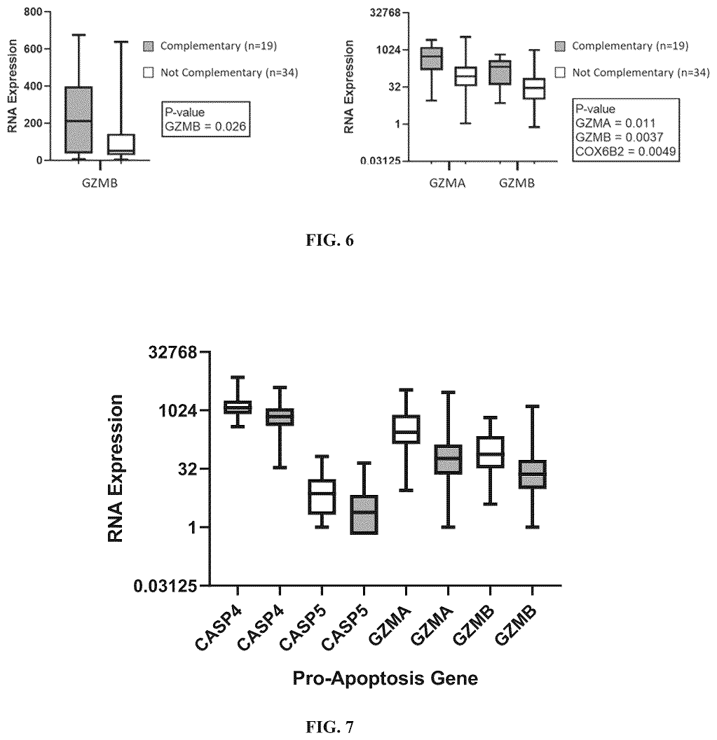

T-cell receptor-α, CDR3-mutanome, chemical complementarity scoring system based on electrostatic charges was then applied to determine whether SARC tumor samples with complementary CDR3-mutanome profiles had an OS rate distinct from the SARC tumor samples representing a lack of electrostatic charge-based complementarity. KM survival analysis indicated that tumors with complementary CDR3-mutanomes represented significantly improved survival compared to tumors with non-complementary CDR3-mutanomes (FIG. 8A). To validate and characterize the survival advantage seen for patients with complementary CDR3-mutanomes, RNASeq values for pro-apoptotic genes were obtained for the tumors representing highly complementary versus the non-complementary CDR3-mutanomes. The samples representing complementary CDR3-mutanomes were found to have significantly increased CASP4, CASP5, GZMA, and GZMB expression compared to all remaining samples, but only GZMB expression was significantly more expressed compared to samples lacking TRA CDR3-mutanome complementarity (FIG. 8B).

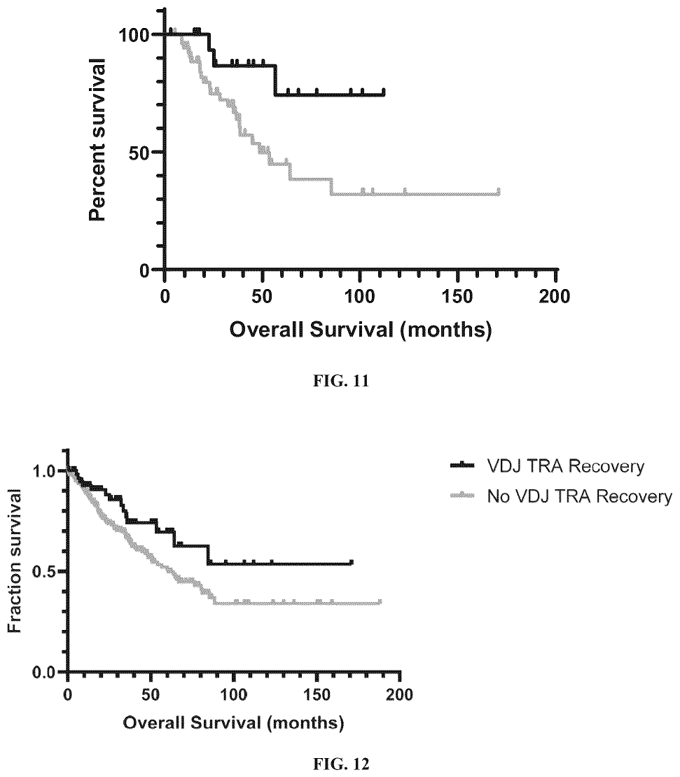

To determine whether CDR3-mutanome complementarity has been a surrogate for a clinical characteristic, clinical data gathered from the TCGA provisional database were first analyzed for OS distinctions using KM curves. Case IDs distinguished by primary pathologic length, multifocal disease, fraction of genome altered, or surgical margin, resulted in an OS distinction on univariate analysis (FIGS. 13A-13D). Independent t-tests were used to determine whether a relationship existed between the complementarity scores and multifocal disease or surgical margin, while Pearson's correlation analysis was performed to determine whether there was a correlation between complementarity score and primary pathologic length or fraction of genome altered. No such relationships were found between these clinical characteristics and complementarity score. Additionally, a cross-tabulation was used to investigate whether CDR3-mutanome complementarity associated with a specific STS subtype. Patients with high complementarity were distributed between two of the largest subtypes in the SARC database, dedifferentiated liposarcoma and leiomyosarcoma (Table 4).

TABLE 4
Cross-tabulation of CDR3-mutant AA complementarity
score distribution between sarcoma subtypes.

| Histologic Subtype | Complementary | All remaining | Total |
|---|---|---|---|
| Leiomyosarcoma | 7 | 98 | 105 |
| Undifferentiated pleomorphic sarcoma | 0 | 51 | 51 |
| Dedifferentiated liposarcoma | 9 | 50 | 59 |
| Myxofibrosarcoma | 0 | 25 | 25 |
| Synovial sarcoma | 0 | 10 | 10 |
| Malignant peripheral nerve sheath tumor | 0 | 9 | 9 |
| Desmoid | 0 | 2 | 2 |
| Total | 16 | 245 | 261 |

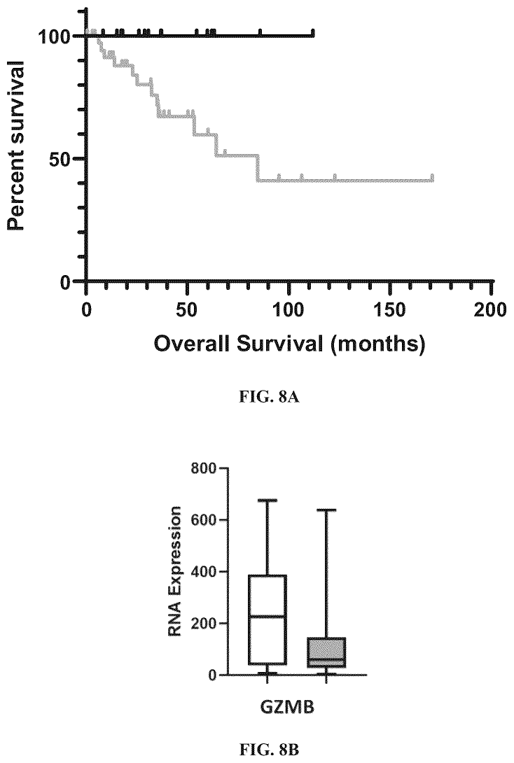

The next assessment was done to analyze particular mutant genes reflecting the survival distinction between patients in the TCGA-SARC database who have TRA recombination read recoveries from their tumor WXS files versus those patients lacking TRA recombination reads. Because TP53 is the most frequently mutated gene in the SARC database (cbioportal.org), the SARC case IDs were first separated into two groups: TP53 mutant and wild-type. KM survival analysis was then performed for the set of case IDs representing mutant TP53, by comparing the OS represented by TRA recombination read recovery versus the OS for lack of TRA recombination read recovery. The KM survival analysis was repeated for case IDs representing wild-type TPS3 Results indicated that, although the OS was significantly improved with TRA recombination read recoveries for the group with mutant TP53 (FIG. 9A), this survival advantage was not seen for the group that had wild-type TP53 (FIG. 9B).

The data described in the above paragraph indicate that CDR3-mutant TP53 AA complementarity can be contributing to the improved OS represented by TRA recombination read recovery (i.e., for the mutant TP53 group). However, due to sample size limitations, an OS distinction based on CDR3-mutant TP53 AA complementarity (versus lack of complementarity) cannot be determined. Thus, the possibility that a dataset of TRA CDR3s represented by RNASeq files was considered, rather than WXS files, would also represent enhanced CDR3 recoveries and the possibility of increased sample size for the CDR3-mutant TPS3 AA calculations and analyses.

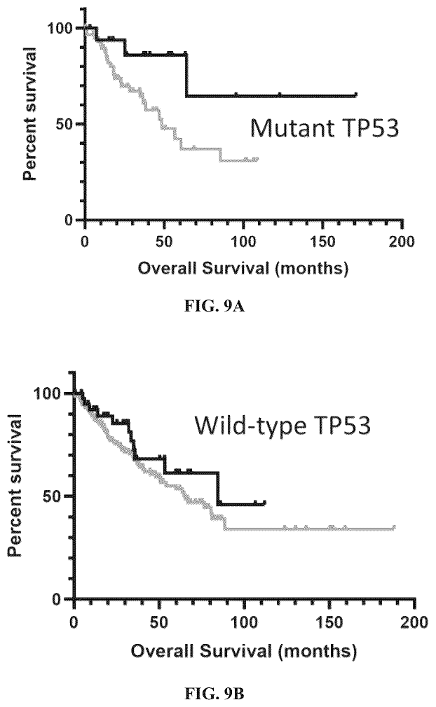

To validate the use of the set of RNASeq-based CDR3s for the TCGA-SARC samples, a KM survival analysis was performed to compare the OS represented by the SARC case IDs representing TRA recombination (RNASeq) read recovery versus all remaining case IDs. Results indicated that the OS represented by RNASeq-based, TRA recombination read recovery was significantly improved compared to the OS represented by all remaining case IDs (FIG. 10A). i.e., this result was consistent with the OS results obtained with the WXS-file based approach for identifying the case IDs with TRA recombination read recoveries (Table 1; FIG. 12).

To further validate the use of the RNASeq-based TRA recombination read recoveries, K M OS analysis for case IDs with mutant and wild-type TP53 was again performed, and for each group the OS represented by RNASeq-based TRA recombination read recovery was compared versus the OS for lack of RNASeq-based TRA recombination read recovery. Within the set of case IDs representing mutant TPS3, survival was significantly improved for those with RNASeq-based TRA recombination read recoveries (FIG. 1013). Again, this survival advantage was not seen for the group that had wild-type TP53 (FIG. 10C), i.e., these results were consistent with results based on the WXS-based approach for comparing OS for case IDs with or without TRA recombination read recoveries, when divided into groups representing either mutant or wild-type TP53.

To investigate whether CDR3-mutant TP53 AA complementarity can be contributing to the improved survival with TRA recombination read recovery in the TP53 group, the CDR3-mutant TP53 AA complementarity was determined using the average NCPRs of the RNASeq-based CDR3s and the corresponding average electrostatic charge of the mutant TP53 AAs KM analysis was performed to compare the OS of SARC case IDs representing complementary and non-complementary CDR3-mutant TP53 AAs. Results indicated a trend towards improved OS for case IDs with complementary CDR3-mutant TP53 AAs, compared to those with non-complementary CDR3-mutant TPS3 AAs (p-value=0.1109; FIG. 10D).

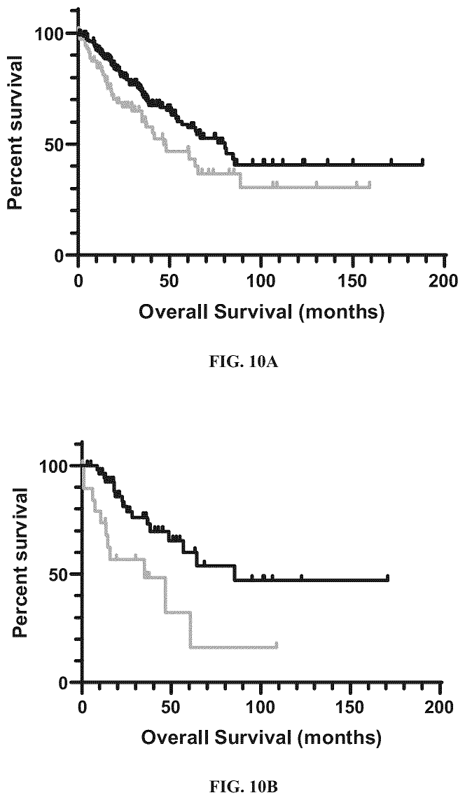

To determine whether a significant survival distinction can be observed by including the next two most frequently mutated genes in the SARC database, the complementarity scores for each patient were calculated using the average NCPRs of the RNASeq-based, TRA CDR3s and the corresponding average electrostatic charge of the mutant TP53, ATRX, and RB1 AAs. KM analysis compared OS of case IDs representing complementary and non-complementary CDR3-mutant TPS3, ATRX, or RB1 AAs. A survival advantage was found for case IDs with complementary CDR3-mutant TP53, ATRX, or RB1 AAs, compared to those with non-complementary CDR3-mutant TP53, ATRX, and RB1 AAs (FIG. 11).

Results of the above analysis, effectively with replicative datasets, i.e., WXS-based CDR3s and RNAseq-based CDR3s, indicate that soft tissue sarcoma patients with electrostatic complementarity between TCR-α CDR3-mutant AAs have an improved OS outcome and more apoptotic tumors. In turn, these results are consistent with the idea that TILs with a chemical attraction to tumor antigens provide the host with improved recognition and destruction of the tumor. Results demonstrating that complementarity between TCR-α CDR3 and the mutant AAs in the sequences of TP53, ATRX, and RB1 is associated with better surviving patients provide support for, and a possible path towards applying electrostatic complementarity in the identification of effective tumor antigens for designing immunotherapies.

Survival analyses of the TCGA database showed that age at diagnosis, primary pathologic length, multifocal disease, fraction of genome altered, and surgical margin all correlated with OS in a univariate analysis. No correlation was found between these clinical characteristics and complementarity score. Thus, TCR-α CDR3-mutant AA complementarity appears to be an independent survival marker.

### Example 4. A Scoring System for the Electrostatic Complementarities of T-Cell Receptors and Cancer-Mutant Amino Acids: Multi-Cancer Analyses of Associated Survival Rates

T-lymphocytes are a key component in the immunological defense against cancers, with the current best understanding being that the most direct component of the anti-cancer immune response is CD8+ T cells recognizing altered-self antigens on HLA class I proteins and lysing tumor cells. This cell killing is mediated by the binding of the T-cell receptor (TCR) to altered-self, otherwise referred to as tumor antigens. While there are extensive correlative data linking immune infiltrates to a high mutation burden, consistent with the expectation that mutant tumor peptides are at the heart of attracting T-infiltrating lymphocytes (TILs) and are thereby collectively stimulating CD8+ T-cell killing, alternative explanations, such as genotoxicity and associated high rates of apoptosis, have not been ruled out as contributors to TIL-mediated, tumor cell killing. Another important question is whether large numbers of mutant peptides provide for a versatile and thereby effective CD8 T-cell response or whether does a large number of mutant peptides simply represent an increased likelihood of one or a few immuno-reactive, effective mutant tumor peptides.

More recently, several tools that have the potential of contributing to a resolution of some of the above issues, have become available. First, bioinformatics and computational approaches have made it clear that chemical and structural features of the TCR CDR3s can correlate with a specific immune response. This development points to the second development, the employment of which offers promise for learning more about specific cancer patient, TCR CDR3, mutant peptide interactions, namely the vast databases now available representing cancer patient mutant AA's and tens of thousands of TCR CDR3's for the corresponding TILs.

In this study, a bioinformatics approach was used to establish the electrostatic features of TCR CDR3s represented by a number of distinct cancer datasets. The TCR sequences used for these analyses were recovered from the cancer genome atlas (TCGA) exome files. These recombined TCR reads have been previously and extensively demonstrated to correspond to a wide variety of tumor cell immune features, used for benchmarking the immunological information from TCR recombination read recovery. That is, TCR recombination reads recovered from tumor specimen exome files represent the dominant TIL species, particularly when there is a large number of patients representing a collective TIL repertoire. Such large patient numbers provide the opportunity to reduce consideration of TCR recombination read outliers and to detect commonalities among subsets of patients. The charge alterations were also assessed represented by corresponding tumor sample mutanomes. Overall, the results below, based on the development of a CDR3, mutant AA complementarity scoring process, indicate that complementary CDR3, tumor mutant AA charges can be used to detect survival rate distinctions among cancer patients, thereby further supporting the idea that increased TIL percentages are linked to increases in TCR-mutant peptide interactions, as opposed to higher TIL percentages having only indirect links to genotoxicity. In addition, the results allow a further understanding of an effective anti-tumor T-cell response. For example, the process below points to specific mutant AA sources that facilitate an anti-tumor immune response, given particular CDR3 chemical features, especially for melanoma, bladder and lung cancer patients.

### Abbreviations

AA, amino acid; BLCA, bladder cancer; CDR3, complementarity determining region-3; DFS, disease free survival; dbGaP, database of genotypes and phenotypes; ESCA, esophageal carcinoma; NCPR, net charge per residue; OS, overall survival; OVCA, ovarian cancer; SKCM, skin cutaneous melanoma; STAD, stomach adenocarcinoma; TCR, T-cell receptor; TCGA, the cancer genome atlas; TILs, tumor infiltrating lymphocytes; WXS, whole exome sequence

### Methods

Recovery of immune receptor V(D)J recombination reads. The Genomic Data Commons (GDC) web portal (portal.gdc.cancer.gov/) was queried for exome (WXS) files available via database of Genotypes and Phenotypes (dbGaP) approved project #6300. A manifest file representing all primary and metastatic tumor WXS binary alignment map (BAM) files available in TCGA was obtained. Slices of the WXS files in which TRA and TRB genes/reads are located were then downloaded to University of South Florida research computing. V(D)J recombination read recovery was performed using a collection of scripts (Computer code package A). The standards for identification of a TRA or TRB recombination read were: (i) a greater than 90% nucleotide match fidelity for both V and J regions, (ii) a 20 nucleotide or greater match length for both V and J regions, and (iii) a CDR3 domain with no stop codons or reading frame shifts.

Assessment of the electric charge of CDR3 regions and tumor mutant amino acids (AA). Recovered immune receptor, V(D)J recombination reads, representing either TRA or TRB, were translated into an AA sequence, and the CDR3 domain, defined at the beginning by the conserved cysteine at the end of all V regions, and at the end by the first AA in the conserved Phe/Trp-Gly-X-Gly J-region motif, was analyzed for net charge per residue (NCPR) using the localCIDER python package (pappulab.github.io/localCIDER/.) “Mutect” mutation data were obtained from GDC. AA substitution mutations were also analyzed for NCPR using the IocalCIDER python package. (Computer code package B).

Assessment of average TCR CDR3-mutant AA complementarity scoresfor each TCGA case ID. An average TCR-mutant AA complementarity score for the tumor sample for each TCGA, cancer dataset case ID (i.e., patient sample set) was calculated in three steps: (i) The change in charge for each AA substitution mutation was multiplied by the NCPR value for every CDR3 recovered, i.e., for both TRA and TRB CDR3s (individually), for a given case ID, yielding an array of possible complementary interactions. A negative score (that is, a positive change in net charge for the mutation multiplied by a negative CDR3 NCPR value, or a negative change in net charge for the mutation multiplied by a positive CDR3 NCPR value) was defined as complementary. A positive score (that is, a positive change in net charge for the mutation multiplied by a positive CDR3 NCPR value or a negative change in net charge for the mutation multiplied by a negative CDR3 NCPR value) was defined as not complementary. The lowest single value of the array of above calculations (i.e. the interaction with the best possible complementarity), for a given mutant AA, whether for the TRA CDR3 or TRB CDR3, was used as the score for that mutation. Note that, often, several distinct TRA CDR3s and several distinct TRB CDR3s were recovered (from the WXS files) for the case IDs. (ii) The most-negative, TCR CDR3-mutant AA complementarity score for every mutation in a given gene was determined as indicated, and the most-negative score was used as the score for that gene for the subsequent case ID average, in the next step, i.e., step (iii); and was used for the calculations of the Methods section below entitled, Assessment of the case ID survival rates associated with specific TCR CDR3-mutant gene complementarity scores. (iii) These lowest scores for each of the mutated genes in the tumor sample of a case ID were averaged across all mutated genes to give that case ID's average TCR CDR3-mutant AA complementarity score. (Computer code package C; see also Table 5, wherein the CDR3 sequences are the ones set forth in SEQ ID NO: 1, SEQ ID NO: 2, SEQ ID NO: 3, SEQ ID NO: 4).

TABLE 6
Example processing output for the correlation of survival rates with complementarity
scores based on individual genes.

| | | TCGA codes for | | | | | | |
|---|---|---|---|---|---|---|---|---|
| | | tissue samples: | | | | | | |
| | CDR3 physical- | 01, primary | | | | | | |
| | chemical | tumor; 06, | Receptor CDR3s | | | | | |
| | property, ncpr = | metastatic | included in | | | Number | Coefficient | Cox |
| TCGA | net charge per | tumor; 10, | complementarity | Mutated | Survival | of | of the Cox | regression |
| dataset | residue. | blood. | score calculations | gene | parameter | samples | regression | p-value |
| SKCM | nepr | 01\|06 | TRA\|TRB | DNAH9 | os | 119 | −4.71894926 | 0.012617665 |
| SKCM | nepr | 01\|06 | TRA\|TRB | MYH1 | os | 113 | −3.31499493 | 0.043057561 |
| SKCM | nepr | 01\|06 | TRA\|TRB | BRAF | os | 156 | −3.40962181 | 0.01754824 |

Assessment of associations of average TCR CDR3-mutant AA complementarity scores with survival rates. Survival rate associations were calculated using an automated python script and the Lifelines python package. (Computer code package C, SOM). Associations between a case ID's survival rate and average TCR CDR3-mutant AA complementarity score, the latter calculated using the three steps indicated above, were assessed using a Cox regression analysis. In addition, independently, survival rate distinctions indicated by the case ID's representing the top and bottom 50^{th} percentiles for the TCR CDR3, mutant AA charge complementarity scores were assessed using a Kaplan-Meier (KM) analysis.

Assessment of the case ID survival rates associated with specific TCR CDR3-mutant gene complementarity scores. Survival distinctions between the following categories were assessed using KM analyses: (a) case ID's representing a mutation in an indicated gene (Results) and an oppositely charged, TCR CDR3 recovered from the corresponding WXS file; (b) case ID's representing a mutation in the same gene and recovery of only non-complementary TCR CDR3s, i.e., TCR CDR3s whereby the only complementarity scores generated, per the above method, were equal to or above zero; (c) case ID's representing a mutation in the same gene and no TCR recombination read recovery from the corresponding WXS file; and (d) case ID's with no mutations in the indicated gene. This process was repeated for every mutated gene in each TCGA cancer dataset studied in this report, using an automated python script and the Lifelines python package (Computer code package D).

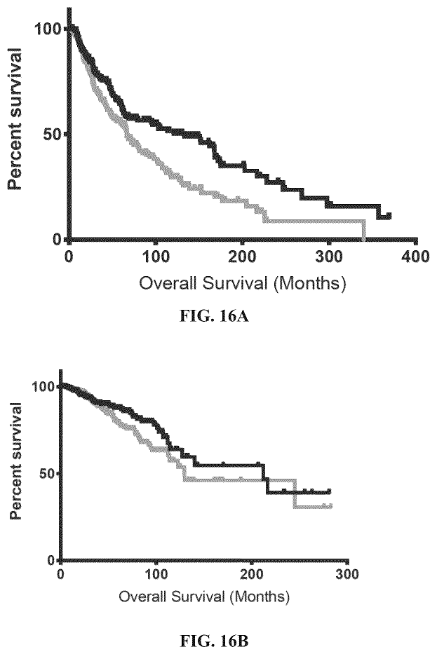

NCPR-based, CDR3-epitope complementarity scoring, benchmarking dataset: First benchmarking approach. The NCPR based complementarity scoring process used in Results was evaluated using a dataset of human CDR3-epitope combinations available at vdjdb.cdr3.net/. All human TRA and TRB CDR3s from this database, along with their cognate epitopes, were processed for the best (most negative) NCPR-based complementarity score for each CDR3-cognate epitope set, exactly as was done for the generation of NCPR based complementarity scores for the survival curves in Results. The only difference is that, in this benchmarking process, the two multipliers in the equation are both NCPRs, i.e., NCPR values for the CDR3s and the matched epitopes. (In Results, below, CS evaluations, the CDR3s have NCPR values, but the tumor samples are represented by a mutant amino acid value, i.e, by the electrostatic charge difference between the mutant amino acid and wild-type amino acid.) Then, the CDR3s representing the best (most negative) NCPR-based CS were randomly re-distributed among the epitopes, to generate random control sets. Five random controls sets were generated. In every case, the average, proper CDR3-epitope NCPR CS was statistically significantly more negative than the re-calculated NCPR CSs following the random sorts of the CDR3 NCPR values (FIG. 18). Overall, the ratio of the proper NCPR-CS calculations to the random calculations was 7.6 to 1. The python code used to generate the data follows the tables of data used to generate FIG. 18. Second benchmarking approach: The above approach was repeated, except in this second approach, there was no pre-determination of the best NCPR-CS for the known CDR3-epitope interactions. Instead, the entire collection of human TRA and TRB CDR3-epitope interactions, available from the vdjdb web tool, was used to calculate the proper NCPR-CSs, for a total of 22,186 pairs. Then, the CDR3 NCPRs were randomized, and the NCPR-CSs were recalculated, as in the first approach, above. In all five cases, the randomized CDR3 NCPR values led to less complementary NCPR-CS (more positive) values with very low p-values firmly establishing statistical significance. The overall ratio of the proper NCPR-CS average, to the random NCPR-CS averages, was 2 to 1.

Online access to code packages A-D. Code packages A-D herein are available at github.com/bchobrut-USF/lgg_idh1.

### Results

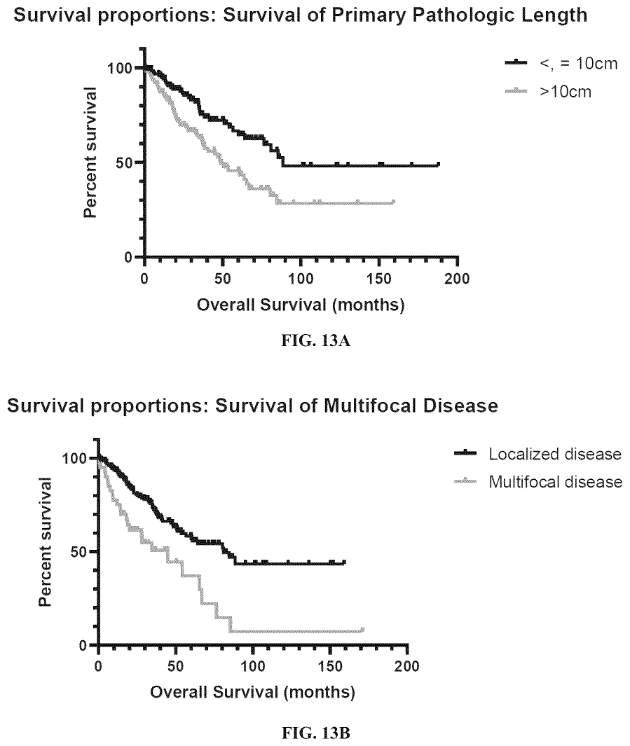

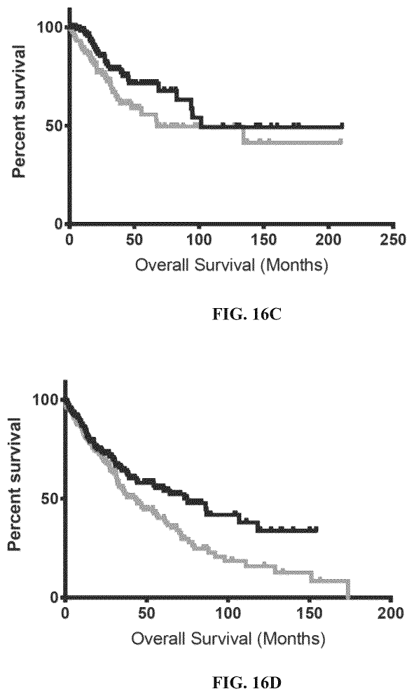

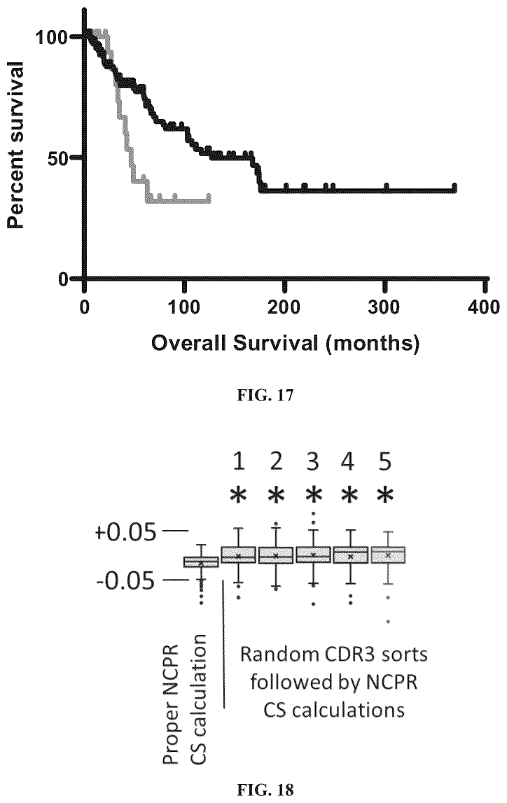

Survival rates and average TCR CDR3-mutant AA complementarity scores, based on CR recombination reads recovered from SKCM tumor specimen exome files. To determine the average electrostatic charge, complementarity scores (Net Charge Per Residue (NCPR)-CS, Methods; FIG. 19 and FIG. 20) for TILs and mutant AAs represented by the TCGA-SKCM case IDs, the mutant AAs and the TRA and TRB recombination reads from the SKCM tumor WXS files were obtained and analyzed. To assess the association between survival rates and TCR CDR3-mutant AA electrostatic complementarity, a Cox regression analysis was used; a higher complementarity (more negative score, Methods; FIG. 19 and FIG. 20) was associated with a significantly better survival (Cox coefficient, −13.01, Cox p=0.00091). The overall survival (OS) of the case ID's representing the higher, electrostatically complementary, 50^{th} percentile of the CDR3-mutant AA scores was compared with the OS of the case ID's representing the lower complementary, 50^{th} percentile of the CDR3-mutant AA scores, using a KM analysis (FIG. 14A). Median survival represented by the upper 50^{th}-percentile of electrostatic charge, complementarity between the TCR CDR3s and mutant AA, was 127.1 months, significantly better than the median survival of 66.69 months represented by the case ID's representing the lower 50m percentile.

The next experiment was performed to assess relationships between survival and complementarity for every mutated gene, individually, in the SKCM dataset, using the TRA and TRB CDR3s of the recombination reads recovered from the SKCM tumor WXS files. As noted in detail in Methods, with this approach, the most-negative complementarity score calculated for any given gene represented the score used to segregate the case IDs into complementary (negative CS for the gene at issue) and non-complementary groups (positive CS for the gene at issue). This approach indicated three genes with statistically significant p-values for both Cox regression and KM analyses, with regard to high complementarity associating with better survival rates: BRAF, DNAH9, and MYH1. BRAF and DNAH9 were further considered here (FIG. 14B; Table 6); MYH1 data are in Table 6. Among the case IDs representing BRAF mutants, those with complementary, CDR3-mutant AA scores less than zero (i.e., high electrostatic complementarity) had the best median survival time (167.58 months), in comparison to several other sets of case ID's, as follows: (a) case IDs representing non-complementary scores (CS>=0, 48.85 months); (b) case IDs representing BRAF mutants but also representing lack of recovery of any TCR recombination reads from the tumor WXS files (53.58 months); and (c) case ID's representing lack of BRAF mutants (66.69 months). Among the case ID's representing DNAH9 mutants, complementary CDR3, mutant AA scores were again associated with higher median survival times (268.53 months, compared to 66.69 months for case IDs representing non-complementary CDR3, mutant AA; and compared to 37.91 months for case IDs for which no TCRs were recovered, and compared to 81.14 months for case IDs with no DNAH9 mutants).

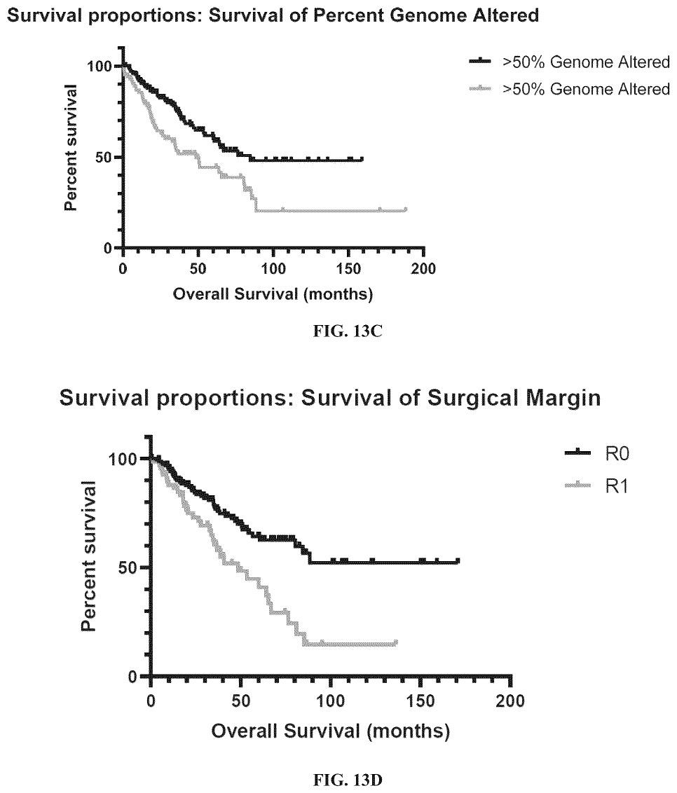

Recovery of 7CR recombination reads from blood WXS files represents survival rate distinctions. An extensive body of work has indicated the value of the recovery of TCR recombination reads from tumor specimen WXS files for correlative studies, as detailed in Introduction. It is also clear that TCR recombinations representing T-cells reactive against cancer antigens can be identified in the blood. In addition, recent work has indicated that blood-exome derived, recombination reads identify V and J usage, HLA allele combinations that strongly correlate with cervical cancer outcomes. This latter result is consistent with the presumed systemic nature of HPV infection, which in turn, for a subset of infected persons, leads to cervical cancer. Thus, the TRA and TRB recombination reads were obtained from all TCGA blood WXS files and determined whether TRA or TRB read recovery, in comparison to no TRA or TRB read recovery, represented a survival distinction, based on both a KM analysis. This approach indicated that survival rates can be distinguished, based on the recovery of the TCR recombination reads, for three cancer datasets (FIGS. 15A to 15C): skin cutaneous melanoma (SKCM), breast cancer (BRCA), and Lung squamous cell carcinoma (LUSC). Thus, the results of FIGS. 15A to 15C justified a role for TRA and TRB recombination reads from blood WXS files for establishing NCPR-CSs associated with survival outcomes. Per below, the usefulness of the blood WXS file, TRA and TRB recombination reads in such associations did indeed become apparent.

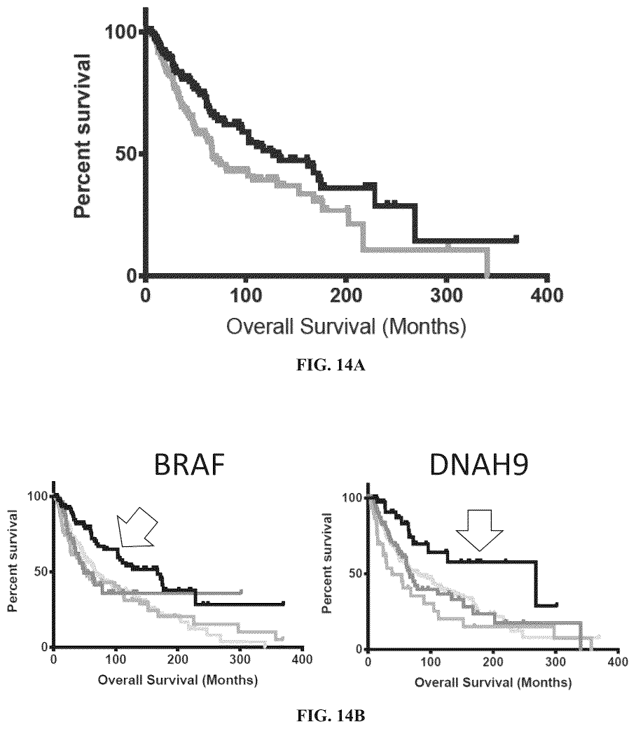

A multi-cancer assessment of survival rate distinctions based on average TCR CDR3-mutant AA complementarity calculations, in turn based on the use of TRA and TRB recombination reads from both blood and tumor WXS files. The results of the SKCM tumor tissue, TCR CDR3-mutant AA complementarity analyses (FIG. 14), and the blood WXS file, TCR-read recovery analyses in the previous section (FIG. 15) indicate that a larger series of survival rate distinctions can be discovered with the CDR3-mutant AA complementarity analyses using recombination reads recovered from either the tumor or blood WXS files (FIG. 16). Using this combined blood and tumor WXS file approach for SKCM, it was determined that the CDR3-mutant AA complementarity was associated with significantly improved survival, using a Cox regression analysis (p-value=0.001256; Cox coefficient, −10.82). The median survival time for the SKCM, upper 50^{th}-percentile complementarity group was 133.4 months, in contrast to the 66.69 months median survival time for the lower 50^{th}-percentile complementarity group (log rank p=0.0012). For the Cox regression analysis of the BRCA dataset, higher complementarity was also significantly associated with better survival (Cox p value=0.030, Cox coefficient, −8.81). For the KM analysis, for BRCA, the more complementary, 50^{th} percentile median survival time was 212.1 months, versus 129.6 months median survival time for the less complementary 50^{th} percentile (log rank p=0.12). For the Cox regression analysis of the cervical squamous cell carcinoma (CESC) dataset, survival rates were significantly better for those case IDs representing the higher complementarity (Cox p=0.018, Cox coefficient, −9.94). Based on the KM analyses, the more complementary 50^{th} percentile of the CESC dataset had a median survival time of 101.7 months, statistically, significantly better than the 67.41-month median survival time of the less complementary 50^{th} percentile (log rank p=0.0496). Cox regression analysis of the LUSC dataset showed significantly better survival with higher TCR-mutant AA complementarity (Cox p value=0.016, Cox coefficient, −9.31). In the LUSC KM analysis, the more complementary-50^{th} percentile had a median survival time of 75.03 months, statistically significantly better than the less complementary 50^{th} percentile, median survival time of 43.20 months (p=0.01).

Multi-cancer identification of specific genes that represent TCR CDR3-mutant AA complementarities, where high complementarity scores represented better survival rates. What was assessed the next was the survival rates associated with gene-specific, complementary CDR3-mutant AA pairs based on recombination reads recovered from both blood and tumor WXS files. This assessment was equivalent to the assessment of FIGS. 14B and 14C, except both blood and tumor WXS files, rather than only tumor WXS files, were used to source the TRA and TRB recombination reads. All TCR CDR3, mutant gene combinations that represented both a Cox regression and KM analysis p-value below 0.05 were identified. Results (Tables 7 and 8) indicated that this approach provided the opportunity to identify TCR CDR3, mutated gene combinations representing cancers that were not previously discoverable as having survival distinctions based on complementarity score differences, using the case ID, average TCR CDR3, mutant AA complementarity score (BLCA, LUAD).

TABLE 7
Specific genes associated with a TCR CDR3, mutant peptide complementarity score for various cancers,
where a high score associated with a high overall survival (OS) rate. Only genes identified with a
sample number above 50, and with both a Cox-regression and OS, log-rank,
KM analysis p-value below 0.05 were included here, with exception of SKCM/BRAF (italics).

| | | | | | | Median | Median | Median |
|---|---|---|---|---|---|---|---|---|
| | | KM | Cox- | N | N | survival, | survival, | survival |
| Cancer | Mutated | analysis | regression | high | low | high | low | difference |
| type | gene | p-value | p-value | (a) | (a) | (months) | (months) | (months) |
| SKCM | DNAH9 | 0.006 | 0.002 | 75 | 91 | 111.01 | 61.47 | 49.54 |
| LUSC | TENMI | 0.016 | 0.014 | 17 | 50 | Undefined | 43.86 | Undefined |
| BLCA | MACFI | 0.014 | 0.034 | 28 | 37 | Undefined | 51.12 | Undefined |
| LUAD | AHNAK | 0.032 | 0.039 | 20 | 42 | Undefined | 37.29 | Undefined |
| SKCM | BRAF | 0.068 | 0.003 | 193 | 31 | 111.01 | 46.78 | 64.23 |

(a) N high and N low refer to number of case ID's representing high and low complementarity for CDR3-mutant AA, respectively.

TABLE 8
Specific genes associated with a TCR CDR3, mutant peptide complementarity score for various cancers,
where a high score associated with a high disease-free survival (DFS) rate.
Only genes identified with a sample number above 50, and with both a Cox-regression and DFS,
log-rank, KM analysis p-value below 0.05 were included here, with exception of SKCM/BRAF.

| | | KM | Cox- | N | N | Median dfs, | Median dfs, | Median dfs |
|---|---|---|---|---|---|---|---|---|
| Cancer | Mutated | analysis | regression | high | low | high | low | difference |
| type | gene | p-value | p-value | (a) | (a) | (months) | (months) | (months) |
| SKCM | TNR | 0.002 | 0.023 | 37 | 50 | 84.63 | 44.15 | 40.48 |
| LUAD | ADAMTS 12 | 0.035 | 0.037 | 26 | 75 | Undefined | 22.47 | Undefined |
| LUSC | PLXNA4 | 0.036 | 0.048 | 16 | 39 | Undefined | Undefined | Undefined |

(a) N high and N low refer to number of case ID's representing high and low complementarity for CDR3-mutant AA, respectively.

Use of CDR3 Sequences Identified in TCGA RNAseq Files as a Replicative Set.

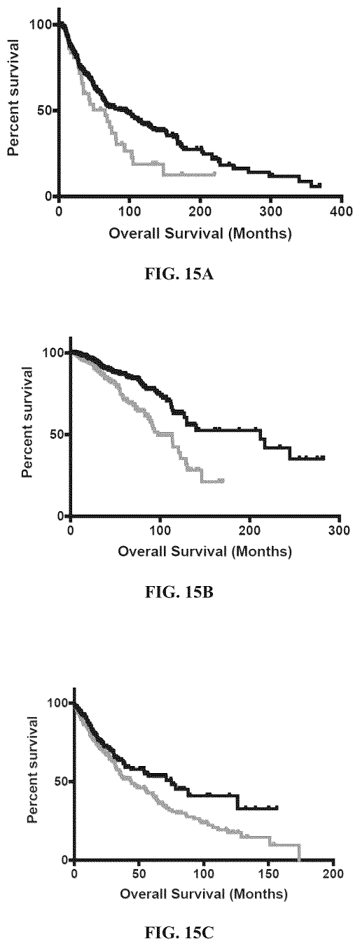

The above data consistently indicated the opportunity to distinguish group survival rates based on the electrostatic complementarity, or lack of complementarity, for patient TRA or TRB CDR3-mutant AA combinations, over multiple datasets. For FIG. 14, where the complementarity analyses were based on TCR CDR3s recovered from WXS files from tumor specimens, it is possible to determine the level of CS calculation consistency using an approximate, replicative dataset, namely the TCR CDR3s recovered from the RNAseq files for the corresponding case IDs. Thus, CSs for each for case ID, for the SKCM mutanome, BRAF, DNAH9, and MYH1 were re-calculated (FIG. 14). The complementary case IDs from that calculation were then mapped onto the case ID sets for the previous complementary calculations using the WXS derived TRA or TRB recombination reads. For examples, in the case of the SKCM mutanome (FIG. 14A), the number of case IDs that remained in the upper 50^{th}-percentile of complementary case IDs weRE determined, after using the RNAseq-recovered CDR3s for the calculation. And, the number of case IDs that represented a negative product was determined (i.e., representing electrostatic complementarity), after using the RNAseq-recovered CDR3s for the BRAF calculations. A summary of the results is in Table 9, strongly supporting the conclusion that CS calculations are consistent using these two different sources of CDR3s. In addition, the CS calculations representing the RNAseq-based CDR3s and mutant BRAF samples also represented a survival distinction, whereby the case IDs with a complementary CS (negative score, Methods; FIG. 19 and FIG. 20) had a better survival rate than the case IDs with a non-complementary CS (FIG. 17).

TABLE 9
Comparing the proportion of complementary CDR3-mutant BRAF, SKCM case IDs
identified with the RNAseq-based CDR3s for either case IDs with complementary
or non-complementary CS based on CDR3s recovered from the WXS files.

| | Proportion of | Proportion of | | |
|---|---|---|---|---|
| | complementary | non-complementary | | |
| | SKCM CDR3- | SKCM CDR3- | | |
| | mutant AA case | mutant AA case | | |
| | IDs identified | IDs identified | | |
| | with the RNAseq- | with the RNAseq- | | |
| | based CDR3s that | based CDR3s that | | |
| | overlap the SKCM | overlap the SKCM | | |
| | WXS-based, | WXS-based, | | |
| | complementary | complementary | | |
| | case ID set (i.e., | case ID set (i.e., | | |
| | the WXS set used | the WXS set used | | n-proportion |
| | in FIG. 14) | in FIG. 14) | 95% CI | test p-value |
| SKCM mutanome | 62.3% | 38.3% | 13-34% | <0.0001 |
| (see also Fig. 14A) | | | | |
| BRAF (see also FIG. | 78.6 | 22.7 | 34-70 | <0.0001 |
| 14B and FIG. 17) | | | | |
| DNAH9 (see also FIG. 14C) | 76 | 4.5 | 56-82 | <0.0001 |
| MYH1 | 74 | 21.3 | 35-66 | <0.0001 |

Large datasets, such as TCGA, have permitted an opportunity to assess the landscape of cancer molecular and biochemical features, and now for the first time, including a landscape of TCR CDR3-mutant AA, complementary NCPR features. The current limitation of landscape goal processes is the ready lack of replicative datasets, particularly until specific landscape features are identified and rise in priority. This is the nature of the process, in that by definition, the molecular and biochemical landscape goals are meant to reach throughout an entire large dataset, again, thereby providing a “no stone left unturned” guidance for the costly, time consuming, and arduous process of establishing and exploiting replicative datasets. However, the above analysis did provide for SKCM tumor-specific replication through the use of CDR3s from SKCM tumor specimen RNAseq files (Table 9 and FIG. 17).

The above analyses are consistent with several possible, important advances, detailed below, particularly keeping in mind that the analyses produced analogous results for the different cancer datasets; and the analyses included significant internal consistencies, such as the BRAF mutant AA, NCPR link to CDR3s, consistent with the fact that that link accounted for a statistically significant, large portion of the linkage of the whole SKCM mutanome to the CDR3 NCPR features.

There has been relatively little direct information that high mutation cancers have a greater percentage of TILs specifically because the TCRs of TILs are reacting with more tumor mutant peptides. While there have been extensive correlations of the TIL percentage and mutation burden in patients, and even in one mouse model, obtaining more specific information related to the putative TCR CDR3, mutant peptide interactions have been challenging. The above data regarding the linkage of improved survival rates to TIL TCR CDR3, mutant peptide electrostatic complementarity (particularly FIG. 14) adds further support to the basic idea of a TIL accumulation due to a response to tumor mutant peptides. Furthermore, the opportunity to identify specific sources of tumor mutant peptides that are electrostatically complementary to TIL TCR CDR3s, given the numerical limitations of the datasets, would suggest that the large number of mutant peptides present in tumors, that is correlated with high TIL percentages, is due to the fact that very large numbers of random mutations eventually hit upon a few suitable tumor mutant peptides, i.e., relatively rare mutant peptides that are able to stimulate TILs. To put it another way, if any or all mutant peptides could function to stimulate T-cells, the above analyses would not have identified specific genes as complementarity partners for the CRD3s due to the lack of statistical significance, in turn due to a lack of the repetitiveness, from patient to patient, of mutant peptide, CDR3 complementarities associated with survival.

With the above approach, in the SKCM dataset, mutant BRAF protein was indicated as potentially providing a consistent mutant, TCR CDR3 electrostatically complementary, peptide (FIG. 14B and FIG. 17). While the above data partitioning did not include assessment of specific BRAF mutations, the overwhelming majority of such BRAF mutations represent the V600E mutant, already known to lead to a strong HLA class I binding peptide and likely relatively resistant to HLA class II proteases that provide HLA class II binding epitopes. Thus, the above data show that longer patient survival is due to the availability of TILs with an electrostatically complementary TCR CDR3. Most importantly, the majority of the BRAF mutants with electrostatic complementary TCR CDR3s overlap the collection of case IDs that represent better survival when the entire mutanome is assessed, in terms of an average CDR3-mutant AA complementarity score (p=0.0002, n-proportion test). Thus, these data are consistent with the idea that only a few mutant peptides represent good stimulators of tumor killing by T-cells.

The other gene sources (Tables 7 and 8), representing specific mutant AA that are electrostatically complementary to corresponding TIL TCR CDR3s have not been substantially, or at all studied empirically, with respect to HLA class I or class II binding or with respect to specific immuno-reactivity, for example, via attempts at ex vivo amplification of TILs. A search of DNAH9 and melanoma in the scientific literature brings up zero entries. Thus, the above described analysis offers the prospect of identifying highly significant T-cell responsive antigens among a very large number of possibilities represented by the entire mutanome.

The above analyses also pointed towards new ways to evaluate an immune response for the purpose of prognoses and for other cancer immunology related purposes, such as evaluating patients for immunotherapy; and evaluating mutant tumor peptides for ex vivo amplification of TILs or for therapeutic vaccinations. As such, the value of the highly cost-effective, exome derivable TCR recombination reads has been extended, and these numerous opportunities argue for the modification of the exon-capture baits in future exome preparations to improve and maximize the information available for tumor immunity studies; or for guiding the therapies of specific patients, particularly in settings where extensive costs for a tumor characterization for an immunotherapy indication are simply not practical. Importantly, the results above establish a new tumor immune state: The presence of TILs but lack of a CDR3-mutant AA complementarity that is apparent in other patients; and not unexpectedly, a concomitant, apparent lack of an effective immune response. Thus, in addition to the two previous states, immunologically cold cancers and cancers with TILs, this third category is useful for stratifying patients, again, for either prognoses goals or therapy guidance. The data point to two fundamentally distinct immunotherapy strategies to extend the lives of most patients: (a) enhancement of a currently successfully CDR3-mutant AA binding affinities, e.g., with immune checkpoint therapy or other immuno-therapies; or (b) augmentation of the patient T-cell repertoire with a T-cell or antibody engineered to bind to whatever antigens are available.

### Example 5. Chemical Complementarity Between Immune Receptor CDR3s and IDH1 Mutants Correlates with Increased Survival for Lower Grade Glioma (LGG)

The immunology of lower grade glioma (LGG) remains complicated, with reports indicating both positive and negative effects of tumor infiltrating lymphocytes. Mutant amino acids in isocitrate dehydrogenase-1 (IDH1), which impair functionality of this enzyme, causing it to produce a possible oncometabolite, 2-hydroxyglutarate, instead of NADPH, are commonly found in low grade gliomas and secondary high grade gliomas. In addition, the common, mutant IDH1-R132H has been considered as a vaccine candidate, with clinical trials ongoing, particularly by using IDH1 peptides representing the IDH1-R132H mutant.

To further understand the role of the IDH1 mutants in the LGG immune response and resolve apparent contradictions for the LGG anti-cancer immune response, the immune receptor recombination reads were obtained from LGG-related exome (WXS) files. The translated CDR3 regions of the immune receptor reads were used for the bioinformatics-based evaluations of interactions between mutant IDH1 and the CDR3s, considered to be the most important part of the antigen binding contact sites for the immune receptors. Tumors representing mutant amino acids that were complementary to (i.e., opposite of) the isoelectric points of the tumor-associated WXS-file recovered immune receptor CDR3 domains, also represented survival distinctions based on that complementarity. For example, bladder cancer tumors with PIK3CA mutants causing a noticeable increase in the isoelectric point were also characterized by a very high survival rate when those tumors also included, due to the TILs, CDR3 domains of T-cell receptor alpha chain (TRA) with low; i.e., complementary, isoelectric points. Furthermore, the same CDR3, isoelectric point-associated survival distinctions were not observed among samples lacking the PIK3CA mutant amino acid.

In addition to the bioinformatics-based advances in bladder cancer tumor immunology that can be applicable to LGG, several reports show the opportunity to use peripheral blood to have a representation of anti-tumor immune responses, including statistically significant survival associations with certain immune receptor recombination reads obtained from blood WXS files and cervical cancer. Therefore, for the below analyses, the TRB and IGH (immunoglobulin heavy chain gene) recombination reads were obtained from both tumor and blood, TCGA-LGG WXS files and the net charge per residue (NCPR) and hydropathy values for the corresponding CDR3 amino acid (AA) sequences have assessed. Assessment of both values allows the consideration of both polar and nonpolar interactions that could be effective in a CDR3-epitope binding reaction. Furthermore, a novel, chemical complementarity scoring system for using both of those parameters was employed to assess the chemical interaction of CDR3 with IDH1 mutants, allowing a clear indication that better survival is associated with a higher level of chemical complementarity of the CDR3s and the IDH1 mutants.

Methods Basic approaches. Recovery of immune receptor recombinations from TCGA-LGG and -SKCM cancer and blood exome files, made available via NIH dbGaP approval number 6300, has been previously and extensively described. The original software for the above processes is available in the supporting online material (SOM, Code packages A, B) and at github.com/bchobrut-USF/lgg_idh1. The CDR3-mutant IDH1 complementarity scoring for NCPR was conducted by multiplying the NCPR value by the change in charge of the mutant AA. For example, an R->H mutation in IDH1 would yield a −1.0 net change in charge at physiological pH, which if multiplied by a CDR3 NCPR of +0.5 would yield a complementarity score of −0.5. A negative number at this step in the calculation denotes a complementary interaction. However, in the next step for generating a final NCPR-complementarity score (CS), values were all multiplied by −1 to maintain consistency with the positive values of the Uversky hydropathy CS calculation, i.e., the positive values of the equivalent Uversky hydropathy calculation represented complementarity. Specifically, to assess hydrophobic interactions, the Uversky hydropathy value of the CDR3 domain was multiplied by the Uversky hydropathy of the mutated amino acid in IDH1. For example, an R->H mutation in IDH1 would yield a +0.144 change in Uversky hydropathy, which if multiplied by a CDR3 Uversky hydropathy of +0.1, would yield a CS of +0.0144. Because amino acid R-groups with high hydrophobicity attract amino acid groups with high hydrophobicity, a more positive Uversky hydropathy CS denotes higher complementarity. The original software for the complementarity scoring process is in the Code package C and at github.com/bchobrut-USF/lgg_idh1. The KM based survival distinctions were obtained using Graphpad Prism. RNASeq expression values were obtained from cBioPortal. Correlations between RNASeq values and OS were calculated using Cox regression. Correlations between RNASeq values and NCPR-CS and Uversky Hydropathy CS were calculated using Pearson's correlation (Code Package D, SOM; github.com/bchobrut-USF/lgg_idh1). Correlations of apoptosis-effector gene RNAseq values and the distinct, TCGA-LGG TRB CDR3-mutant IDH1, NCPR-based complementarity and noncomplementarity groups, respectively, were assessed with a Mann Whitney U test (Code Package E; github.com/bchobrut-USF/lgg_idh1)

Code availability. github.com/bchobrut-USF/lgg_idh1.

Methods, with references and a complementarity scoring benchmarking dataset-Recovery of immune receptor V(D)Jrecombination reads. The Genomic Data Commons (GDC) web portal (portal.gdc.cancer.gov/) was queried for exome (WXS) files available via database of Genotypes and Phenotypes (dbGaP) approved project #6300. A manifest file representing all primary and metastatic tumor WXS binary alignment map (BAM) files available in TCGA was obtained. Slices of the WXS files in which TRA and TRB genes are located were then downloaded to University of South Florida research computing. V(D)J recombination read recovery was performed using a collection of scripts similar to previous publications (Computer code package A). The standards for identification of a TRA or TRB recombination read were: (i) a greater than 90% nucleotide match fidelity for both V and J regions, (ii) a 20 nucleotide or greater match length for both V and J regions, and (iii) a CDR3 domain with no stop codons or reading frame shifts.

Assessment of the electric charge of CDR3 regions and tumor mutant amino acids (AA). Recovered immune receptor, V(D)J recombination reads, representing either TRA or TRB, were translated into an AA sequence, and the CDR3 domain, defined at the beginning by the second cysteine in the V region, and at the end by the first AA in the conserved Phe/Trp-Gly-X-Gly J-region motif, was analyzed for net charge per residue (NCPR) using the localCIDER python package (pappulab.github.io/localCIDER/.) Mutect mutation data were obtained from GDC. AA substitution mutations were also analyzed for NCPR using the localCIDER python package (Computer code package B).

Assessment of average TCR CDR3-mutant AA complementarity scores for each TCGA case ID. An average TCR-mutant AA complementarity score for the tumor sample for each TCGA, cancer dataset case ID (i.e., patient sample set) was calculated in three steps: (i) The change in charge for each AA every substitution mutation was multiplied by the NCPR value for every CDR3 recovered, i.e., for both TRA and TRB CDR3s (individually), for a given case ID, yielding an array of possible complementary interactions where a negative score (that is, a positive change in net charge for the mutation multiplied by a negative CDR3 NCPR value, or a negative change in net charge for the mutation multiplied by a positive CDR3 NCPR value) was defined as complementary and a positive score (that is, a positive change in net charge for the mutation multiplied by a positive CDR3 NCPR value or a negative change in net charge for the mutation multiplied by a negative CDR3 NCPR value) was defined as not complementary. The lowest single value of the array of above calculations (i.e. the interaction with the best possible complementarity), for a given mutant AA, whether for the TRA CDR3 or TRB CDR3, was used as the score for that mutation. Note that, often, several distinct TRA CDR3s and several distinct TRB CDR3s were recovered (from the WXS files) for the case IDs. (ii) The most-negative, TCR CDR3-mutant AA complementarity score for every mutation in a given gene was determined as indicated, and the most-negative score was used as the score for that gene for the subsequent case ID average, in the next step, i.e., step (iii); and was used for the calculations of the Methods section below entitled, Assessment of the case ID survival rates associated with specific TCR CDR3-mutant gene complementarity scores, in comparison with other case ID categories. (iii) These lowest scores for each of the mutated genes in the tumor sample of a case ID were averaged across all mutated genes to give that case ID's average TCR CDR3-mutant AA complementarity score. (Computer code package C).

Assessment of associations of average TCR CDR3-mutant AA complementarity scores with survival rates. Survival rate associations were calculated using an automated python script and the Lifelines python package. (Computer code package C). Associations between a case ID's survival rate and average TCR CDR3-mutant AA complementarity score, the latter calculated using the three steps indicated above, were assessed using a Cox regression analysis. In addition, independently, survival rate distinctions indicated by the case ID's representing the top and bottom 50th percentiles for the TCR CDR3, mutant AA charge complementarity scores were assessed using a Kaplan-Meier (KM) analysis.

Assessment of the case ID survival rates associated with specific TCR CDR3-mutant gene complementarity scores, for comparison with other case ID categories. Survival distinctions between the following categories were assessed using KM analyses: (a) case ID's representing a mutation in an indicated gene and an oppositely charged, TCR CDR3 recovered from the corresponding WXS file; (b) case ID's representing a mutation in the same gene and recovery of only non-complementary TCR CDR3s, i.e., TCR CDR3s whereby the only complementarity scores generated, per the above method, were equal to or above zero; (c) case ID's representing a mutation in the same gene and no TCR recombination read recovery from the corresponding WXS file; and (d) case ID's with no mutations in the indicated gene. This process was repeated for every mutated gene in each TCGA cancer dataset studied in this report, using an automated python script and the Lifelines python package (Computer code package D).

NCPR-based, CDR3-epitope complementarity scoring, benchmarking dataset-First benchmarking approach: The NCPR based complementarity scoring process used in this article was evaluated using a dataset of human CDR3-epitope combinations available at vdjdb.cdr3.net/. All human TRA and TRB CDR3s from this database, along with their cognate epitopes, were processed for the best (most negative) NCPR-based complementarity score for each CDR3-cognate epitope set, exactly as was done for the generation of NCPR based complementarity scores for the survival curves in the article. The only difference is that, in this benchmarking process, the two multipliers in the equation are both NCPRs, i.e., NCPR values for the CDR3s and the matched epitopes. (In the article CS evaluations, the CDR3s have NCPR values, but the tumor samples are represented by a mutant amino acid value, i.e, by the electrostatic charge difference between the mutant amino acid and wild-type amino acid.) Then, the CDR3s representing the best (most negative) NCPR-based CS were randomly re-distributed among the epitopes, to generate random control sets. Five random controls sets were generated. In every case, the average, proper CDR3-epitope NCPR CS was statistically significantly lower than the re-calculated NCPR CSs following the random sorts of the CDR3 NCPR values (FIG. 19). Overall, the ratio of the proper NCPR-CS calculations to the random calculations was 7.6 to 1. The python code used to generate the data follows the tables used to generate FIG. 19.

Second benchmarking approach: The above approach was repeated, except in this second approach, there was no pre-determination of the best NCPR-CS for the known CDR3-epitope interactions. Instead, the entire collection of human TRA and TRB CDR3-epitope interactions, available from the vdjdb tool, was used to calculate the proper NCPR-CSs, for a total of 22,186 pairs. Then, the CDR3 NCPRs were randomized, and the NCPR-CSs were recalculated, as in the first approach, above. In all five cases, the randomized CDR3 NCPR values led to less complementary NCPR-CS (more positive) values with very low p-values firmly establishing statistical significance. The overall ratio of the proper NCPR-CS average, to the random NCPR-CS averages, was 2 to 1.

### Results

TRB, CDR3-mutant IDH1 complementarity scores and associated survival rates. To assess complementarity between TCR CDR3 domains and IDH1 mutant amino acid (AA) substitutions, two separate, complementarity scoring systems were developed for evaluating electrostatic and hydrophobic interactions, respectively. To calculate the electrochemical complementarity score (CS), the net charge per residue (NCPR) value of the CDR3 AA sequence was multiplied by the charge change due to the mutant AA in IDH1. That is, the NCPR-CSs were computed with a patient's IDH1 mutant AA, as one factor, and each of their TRB CDR3 AA sequences, respectively, as the other factor, in separate calculations, with the NCPR-CS result representing the highest complementarity for a given case ID used in all subsequent analyses in this report. The TRB CDR3 AA sequences were obtained from the tumor and blood WXS file searches. Tumor TRB recombination read counts were insufficient, on their own, in the case of LGG, to perform NCPR-CS-based survival analyses. Thus, the TRB recombination reads were included from the LGG blood WXS files as well: (a) the ready detection of serum glioma-derived biomarkers, including IDH1 transcripts associated with glioma; and (b) the ready detection of immune receptors in blood directed against tumor antigens. More specifically, the opportunity to exploit the blood WXS files, for survival related, immune receptor recombination reads have been established for cervical cancer. In sum, the greatest complementary, NCPR-CS was obtained for every TCGA-LGG case ID, where there was recovery of at least one TRB recombination read from either the blood or tumor WXS file.

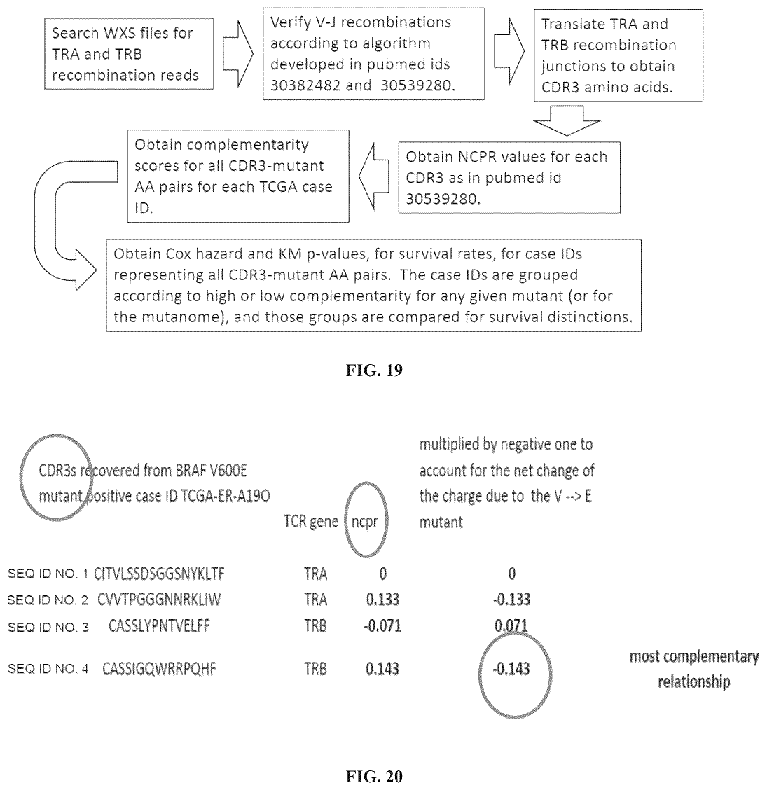

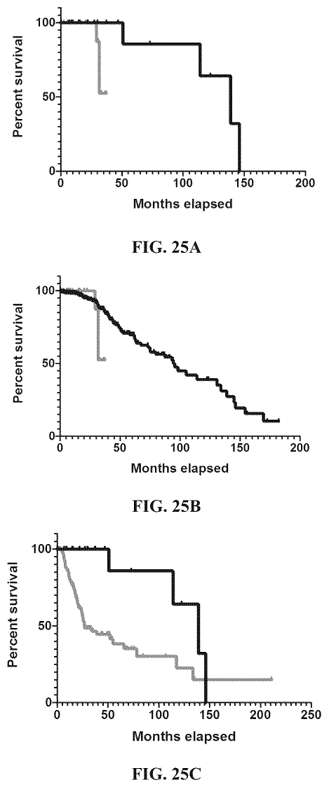

Case IDs representing high TRB-mutant IDH1 complementarity, due to having at least one of their TRB, CDR3-mutant IDH1 NCPR-CS greater than zero, with greater than zero identifying a mathematical representation of a complementary interaction, were compared to case IDs without a complementary TRB, CDR3-mutant IDH1 interaction (final NCPR-CS calculation result of less than or equal to zero), using Kaplan-Meier (KM) survival analysis (FIG. 21A). Patients with complementary TRB, CDR3-mutant IDH1 NCPR-CSs showed a significantly better overall survival (OS) (median survival 138.93 months) compared to those without a complementary CS (median survival 73.42 months) (p=0.0083)(univariate Cox regression p-value=0.029). Case IDs with complementary TRB, CDR3-mutant IDH1 NCPR-CSs also showed a significantly better OS rate in comparison to all remaining TCGA-LGG case IDs (FIG. 26).

To extend and further validate the relationship of the NCPR-CS calculations to OS, several approaches were employed. Because the TCGA-LGG set has not been duplicated, allowing replication of the result using a replicative dataset, the TRB CDR3-mutant IDH1, LGG set was randomly divided in half; that half, the complementary and noncomplementary case IDs were then collected; and the KM analyses was then re-conducted, obtaining similar results (p=0.0169), i.e., the complementary NCPR-CS scores represented a better OS rate. Second, the NCPR-CS scoring approach was applied using TRB CDR3s derived from a separate approach and dataset, namely using CDR3s obtained from RNASeq files. To exploit this RNASeq-based, CDR3 recovery approach, the TCGA-SKCM (melanoma) dataset for NRAS mutants was employed, because this SKCM dataset represented a sufficient number of TRB recombination reads from the tumor samples alone, a necessary consideration because there are no RNASeq files for any TCGA blood samples that would permit TRB recombination read mining. Results indicated that complementary NCPR-CS values represented a higher OS rate (FIG. 21B), and this result was in turn verified by the higher OS rate represented by complementary NCPR-CS values calculated using the TRB recombination read recoveries from the TCGA-SKCM WXS files representing the NRAS mutants (FIG. 21C).

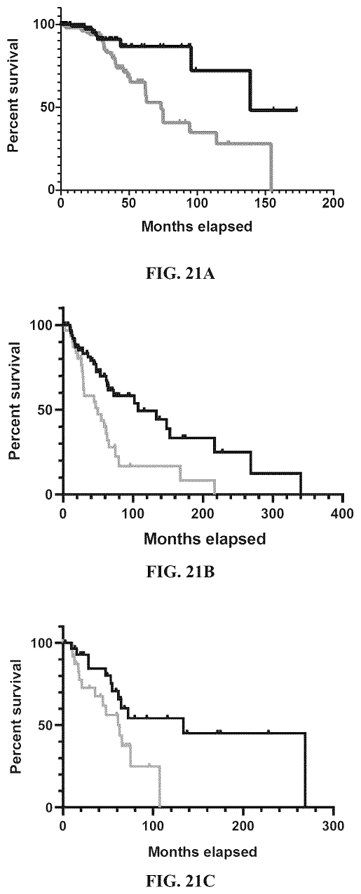

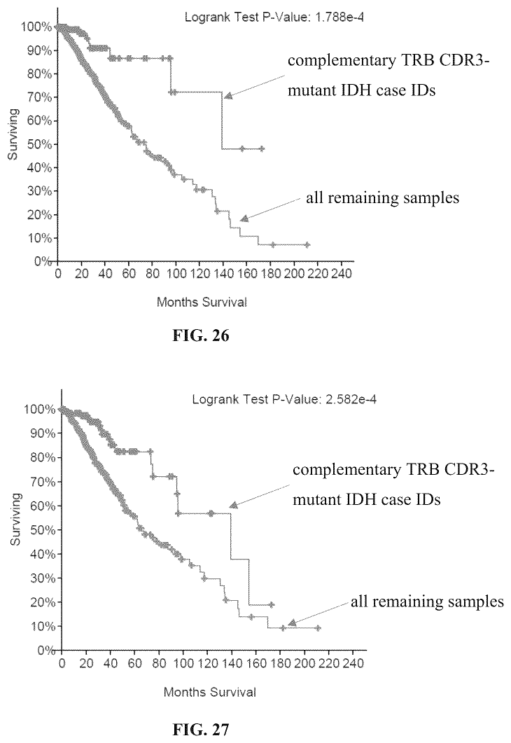

Next, a distinct chemical approach was applied to the LGG dataset. A Uversky hydropathy CS was generated for each case ID with mutant IDH1. OS rates for case IDs with a Uversky hydropathy CS in the top 50^{th} percentile were then compared to survival rates for case IDs with a Uversky hydropathy CS in the bottom 50^{th} percentile, using the KM analysis (FIG. 22A. Case IDs representing a high Uversky hydropathy CS had a significantly better OS rate (median survival 138.93 months) compared to those with a relatively low Uversky hydropathy CS (median survival 62.12 months) (KM p=0.025; a univariate Cox regression p-value=0.059). (This result was replicated by randomly dividing the above Uversky hydropathy, CS set in half and repeating the KM analysis for the complementary and noncomplementary case IDs within that randomly produced, half set: p=0.0056) Case IDs with a high Uversky hydropathy CS also showed a significantly better survival rate in comparison to all remaining TCGA-LGG case IDs (FIG. 27).

Complementarity scoring as an apparent, independent indicator of survival rates. To evaluate the relationship of CSs, for overall survival, in the setting of clinical features, a multivariate, Cox proportional hazards model (evaluated with statistical packages for the social sciences, version 25), including the TRB-based NCPR-CS and Uversky CS, race, tumor grade, age, gender and mutation, revealed a significant inverse correlation with survival, with complementarity, for both the NCPR and Uversky-hydropathy scoring methods only (Table 10). This model also revealed a significant correlation with death for age, but none of the other, above clinical features represented any significance.

TABLE 10
Multivariate analysis for NCPR- and Uversky
hydropathy-CS and clinical parameters.

| Co-variates | B | Significance |
|---|---|---|
| NCPR-CS | −5.00 | 0.039 |
| Uversky hydropathy-CS | −34.65 | 0.016 |
| Age | 0.058 | <0.001 |

Combining the NCPR-CS and Uversky hydropathy CS calculations. To assess the survival correlation after combining the values of the NCPR- and Uversky hydropathy-mutant IDH1 CS, the two sets of CSs were z-scored, and the average of each case ID's NCPR-CS z-score and Uversky hydropathy CS z-score was obtained. Case IDs representing the top 50^{th} percentile, combined CS were then compared to those representing the bottom 50^{th} percentile, combined CS using the KM analysis (FIG. 22B). Case IDs representing the more complementary TRB CDR3-mutant IDH1, combined CS represented a significantly better OS (median survival, undefined) compared to those with the lessor TRB CDR3-mutant IDH1, combined CSs (median survival 62.12 months) (KM, p=0.0003; a univariate Cox regression p-value=0.0026).

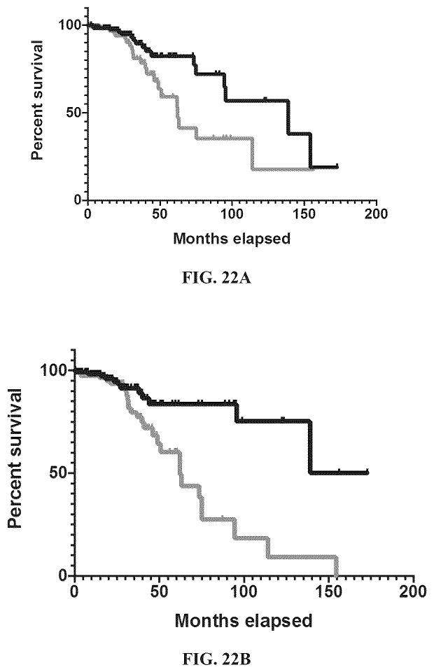

Lack of complementarity is not distinguishable from lack of TRB recombination read recovery. To compare the overall survival rates for case IDs with no TRB recombination read recoveries to case IDs with non-complementary CSs, a KM analysis was conducted, as follows. Case IDs with an IDH mutation and with a TRB CDR3-mutant IDH1 NCPR-CS less than or equal to zero; i.e., a non-complementary CS calculation (median survival 73.42 months) represented the same overall survival rate as case IDs with an IDH1 mutation and no TRB recombination read recoveries (median survival 93.13 months) (FIG. 23A, p-value=0.3374). Case IDs representing an IDH1 mutation and a Uversky hydropathy-mutant IDH1 CS in the bottom 50^{th} percentile (median survival, 62.12 months) had the same overall survival rate, from the standpoint of lack of statistical significance, as did case IDs where there was no TRB recombination read recovery (FIG. 23B, p-value=0.2656); i.e., the same set of case IDs referred to above (FIG. 23A) for a comparison with the case IDs with a NCPR-CS equal to or less than zero. And finally, case IDs representing the bottom 50^{th} percentile for the combined NCPR, Uversky hydropathy CS (median survival 62.12 months) also did not have a significantly different overall survival rate than the overall survival rate represented by case IDs with an IDH1 mutation and no TRB recombination read recoveries (FIG. 23C, p-value=0.0501).

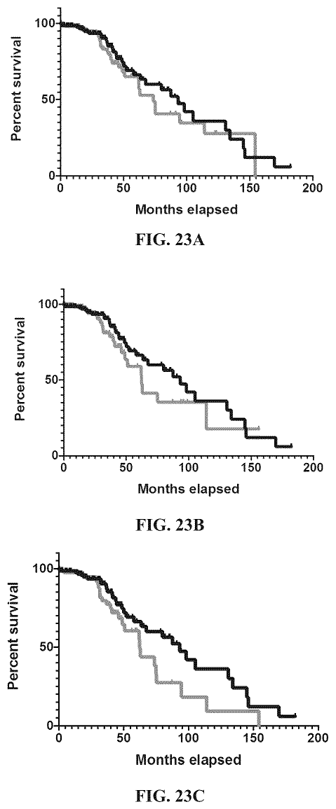

A comparison of CDR3-mutant IDH1 complementarity and lack of the IDH1 mutation. It has been clear for quite some time that the lack of an IDH1 mutation represents a poor prognosis, particularly for LGG. Therefore, it is of practical value to understand the added value for prognosis by factoring in the above indicated, complementary CDR3-mutant IDH1 CSs. Thus, to compare the overall survival rate represented by case IDs with a complementary TRB CDR3-mutant IDH1 CS to the overall survival rates represented by case IDs representing a lack of an IDH1 mutation, a KM analysis was conducted. The overall survival rates represented by case IDs with an IDH1 mutation and a complementary (i.e., greater than zero) NCPR-CS (median survival 138.93 months) were significantly higher than the overall survival rates represented by case IDs lacking an IDH1 missense mutation, regardless of whether there was a recovery of a TRB recombination read from the corresponding WXS files (median survival 26.91 months) (FIG. 24A, p-value<0.0001). Case IDs with an IDH1 mutation and a Uversky hydropathy-mutant IDH1 CS calculation in the top 50^{th} percentile (median survival 138.93 months) demonstrated significantly higher survival rates than patients without an IDH1 mutation, again, regardless of whether there was a recovery of TRB recombination reads (median survival 26.91 months) (FIG. 24B, p-value<0.0001). And, similarly consistent results were obtained for survival rates representing case IDs with a high (top 50^{th} percentile) z-score-based, combined, NCPR, Uversky hydropathy CS (median survival, undefined) (FIG. 24C, p-value<0.0001).

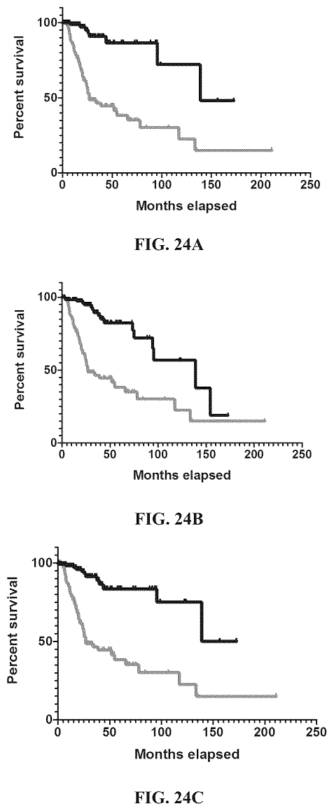

IGH, CDR3-mutant IDH1 complementarity scores and associated survival rates. A similar complementarity scoring approached was used for assessing potential IGH CDR3-mutant IDH1 relationships, using both the NCPR and Uversky hydropathy values. The IGH CDR3-mutant IDH1 NCPR-CSs were not linked to significant survival differences, when evaluating the case IDs representing complementary (greater than zero) and non-complementary NCPR-CSs (equal to or below zero). However, the Uversky hydropathy-based, CDR3-mutant IDH1 CSs representing a complementary interaction (top 50^{th} percentile) were linked to significantly better OS rates (median survival, 138.93 months), compared to the case IDs represented by CDR3-mutant IDH1 CSs with low complementarity (bottom 50^{th} percentile) (median survival, undefined) (FIG. 25A; p-value=0.0167; A univariate Cox regression p-value=0.059). Furthermore, there was no significant difference in the OS rates represented by case IDs in the bottom 50^{th} percentile of the Uversky hydropathy CS calculations and case IDs where there were no IGH recombination read recoveries from the WXS files (FIG. 25B, p-value=0.096).

Once again, given the significantly poorer prognosis of LGG cases lacking an IDH1 mutation, analysis was performed to understand the opportunities for patient distinctions by comparing the overall survival rates of case IDs representing the upper 50^{th} percentile of Uversky hydropathy, IGH CDR3-mutant IDH1 and the case IDs lacking an IDH1 mutation. Thus, the former set of case IDs represented a significantly better overall survival rate (median survival 138.93 months) than patients without an IDH1 mutation, regardless of TRB or IGH recovery status (median survival 26.91 months). (FIG. 25C, p-value<0.0046).

Gene expression results associated with complementarity scores. To identify gene expression values linked to the TRB CDR3-mutant IDH1 complementarity scores, either to explain the associated distinct survival rates or to inform other approaches to distinguishing survival rates, a method was scripted for identifying genes whose expression was associated with either a poor or good complementarity score, for both the NCPR-CSs and the Uversky hydropathy, TRB CDR3-mutant IDH1 CSs. Only four genes met the standards of RNASeq-based expression values independently representing overall survival distinctions, analyzed by Cox regression, and had a significant Pearson's correlation co-efficient, for a correlation with the CS scores (Table 11). Of these four, only one, IL17RC, had a connection to immune functions, with the IL17RC high expression levels inversely correlating with high complementarity.

TABLE 11
Four genes representing independent associations with LGG overall survival and correlating,
or inversely correlating with CDR3-mutant IDH1 complementarity scores.

| | | | | Uversky | |
|---|---|---|---|---|---|
| | | OS p-value | NCPR-CS, | hydropathy-CS, | |
| | | from cox | Pearson's | Pearson's | |
| | | regression | correlation | correlation | |
| | | analyses, for | p-values for | p-values for | |
| | | associations | associations | associations | |
| | Overall survival | indicated in | indicated in | indicated in | |
| | and | first and | first and | first and | |
| | complementarity | second | second | second | |
| Gene | association | columns | columns | columns | Comments/Function |
| SGCG | Upregulated with | 0.0346 | 0.0251 | 0.0038 | Dystrophin-interacting |
| | OS and high | | | | protein |
| | complementarity | | | | |
| PER3 | Upregulated with | 0.0157 | 0.0207 | 1.71E−05 | Related to circadian |
| | OS and high | | | | rhythm, expressed in |
| | complementarity | | | | the suprachiasmatic |
| | | | | | nucleus |
| IL17RC | Down-regulated | 0.0018 | 0.0103 | 0.0055 | Homology with the |
| | with OS and high | | | | I17RA, but expressed |
| | complementarity | | | | in nonhemopoietic |
| | | | | | tissues |
| TTC13 | Down-regulated | 0.0316 | 0.0085 | 0.04567 | Unknown function |
| | with OS and high | | | | |
| | complementarity | | | | |

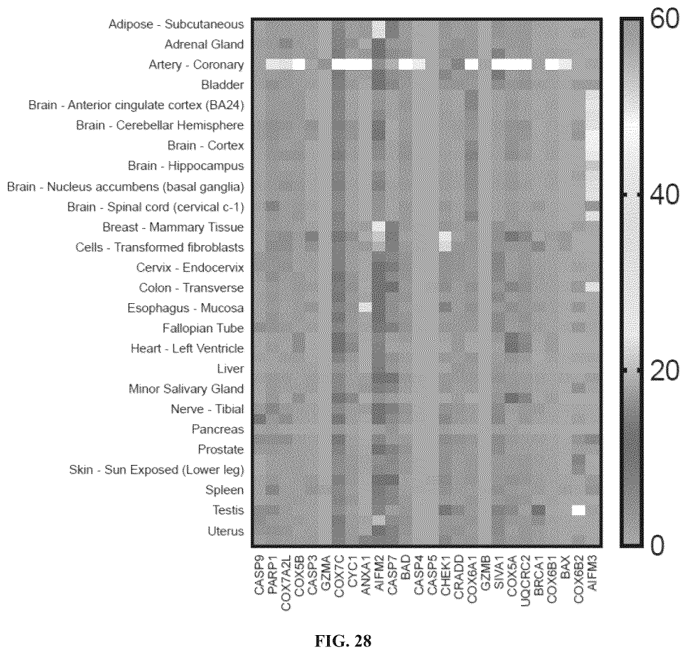

To further investigate the possibility of gene expression differences reinforcing the overall survival distinction represented by complementary and noncomplementary scores for the LGG dataset, the RNASeq values was assessed for a previously described set of apoptosis-effector genes extensively linked to cancer cell viability. However, a Mann-Whitney U test was used to establish statistically significant differences in this second approach for assessing the difference for the complementary versus noncomplementary groups represented by the NCPR-CSs alone. Results of this analysis indicated a p-value below 0.05 for only two apoptosis effector genes. One of these genes, AIFM3, was expressed at a higher level in the tumor samples represented by the complementary NCPR-CS values (an average of 322 versus an average of 267 units). In addition, AIFM3 expression showed a correlation trend with the NCPR-CS scores, with a Pearson's correlation's p-value of 0.08. Strikingly, AIFM3 has been previously shown to be expressed at a dramatically high level in brain tissue, and to be expressed at little more than background levels in all other tissues among a large panel of tested tissue (GTEx Portal; FIG. 28). In addition, all of the other apoptosis effector genes assessed above, have been shown to be expressed only at very low levels in the brain (FIG. 28).

The above analyses are consistent with the conclusion that a good binding interaction between the available TRB CDR3s, or the IGH CDR3s, and a mutant peptide representing IDH1 leads to a longer patient survival. This specificity of immune responsiveness in LGG is consistent with reports that mutant IDH1 has shown promise as a tumor vaccine and with reports that IDH1 mutant positive tumors have distinct immune responses. And thus, the results above help to understand differing reports as to the role of the immune system in LGG. With the indicated IDH1 mutant specificity, detected with two bioinformatics-based, chemical parameters, there is a functional and beneficial immune response. But with lack of such specificity, the immune response is either irrelevant or may do more harm than good (FIG. 23). Furthermore, these distinctions are independent of mutation burdens, i.e., low and high NCPR-CS, TCGA-LGG groups have equivalent average mutation burdens. And, these CS-based overall survival distinctions are specifically independent of mutations in the top three mutated genes in the TCGA-LGG dataset, namely TP53, ATRX, and CIC.

Given the reports showing the immunogenicity of mutant IDH1, the above results, particularly related to patient survival rates, indicate that a high CDR3-mutant IDH1 CS represents TRB and IGH binding to a mutant IDH1 peptide, along with an immune response against tumors displaying that peptide, in the case of TRB, on HLA molecules.

The above example shows a method for more reliable prognoses, particularly in attempting to inform the extremes of: (a) lacking an IDH1 mutation; and (b) having an IDH1 mutation with corresponding, chemically complementary immune receptor CDR3s. Also, such distinctions guide therapy, including, for example, determining and/or predicting whether a checkpoint blockade approach can be more effective in a patient with high CDR3-mutant IDH1 complementarity scores; determining and/or predicting CAR T-cells or therapeutic antibodies, bringing their own antigen-binding immune receptor to the tumor, be more effective with a low CDR3-mutant IDH1 CS; and, finally, designing therapeutic antibody or CAR T-cell receptor engineering with improved complementary IGH CDR3-mutant IDH1 CSs.

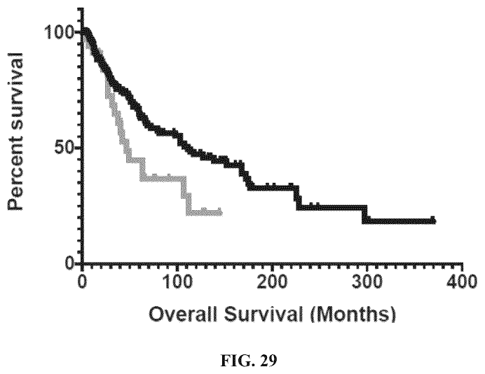

Further to all of the above complementary scoring approaches related to the T-cell receptor, made use of TRB CDR3 amino acid sequences, analogous approaches with TRA-based CDR3 amino acid sequences was also applied. TRA CDR3 is known to contact with T-cell receptor epitopes. The data disclosed herein shows that TRA CDR3-mutant BRAF NCPR-CSs, based on TRA recombination reads obtained from blood and tumor WXS files, defined distinct overall survival rates (FIG. 29).

### Example 6. Electrostatic Complementarity of B-Cell Receptor CDR3s and TP53 Mutant Amino Acids in Breast Cancer is Associated with Increased Disease-Free Survival Rates

### Results

Breast cancer has long been characterized as a B-cell related disease, with regard to the immune response. For example, data indicate that B-cell receptor (BCR) recombination read recoveries from breast cancer exome (WXS) files represent a higher disease-free survival (DFS) rate. However, antigens stimulating the B-cell response have not been well substantiated, particularly via immunogenomics analyses. Thus, the net charge per residue (NCPR) was evaluated for CDR3s from BCRs from tumor infiltrating lymphocytes (TILs) to assess electrostatic complementarity with TP53 mutant amino acids (AAs), because the CDR3 loop domain is the most important part of the BCR polypeptide for antigen binding specificity.

Two methods, termed the extended and abbreviated approaches, were used to develop complementarity scores (CSs), based on CDR3-mutant TP53 AA electrostatic complementarities. Both methods assessed complementarity using NCPR for the BCR CDR3s and a value for TP53 mutants based on the difference between the amino acid charges of the mutant and wild type TP53.

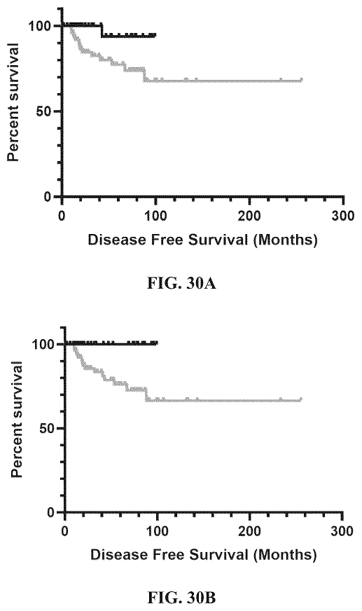

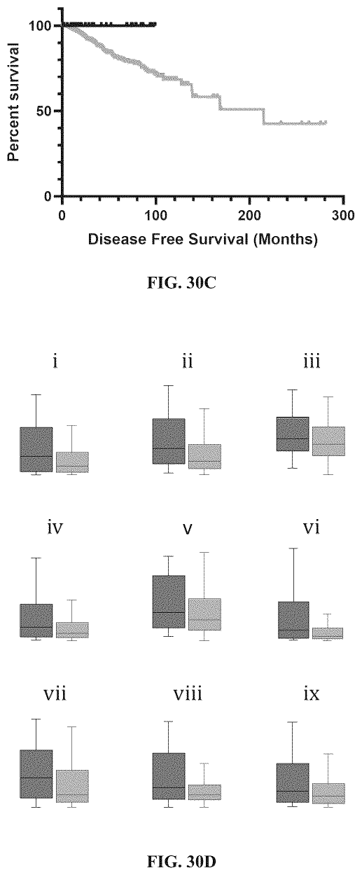

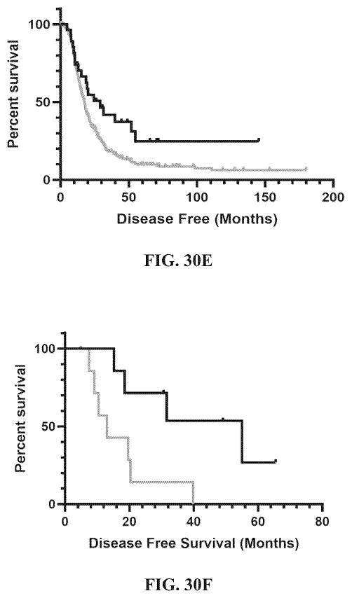

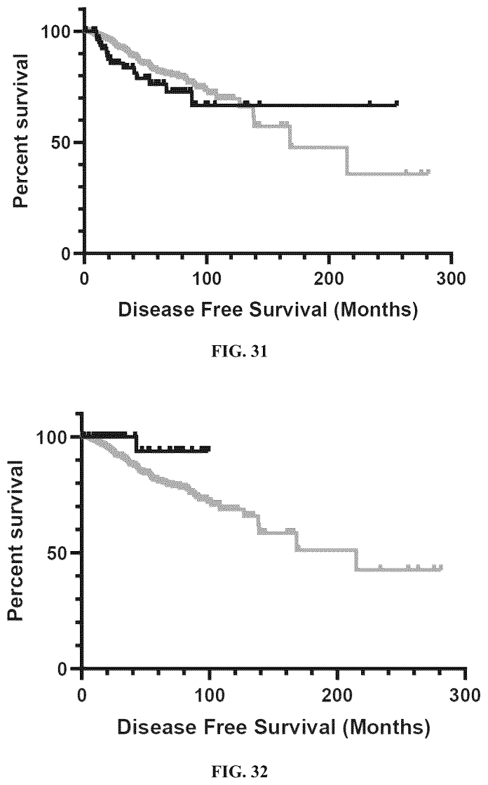

Kaplan Meier (KM) analysis of DFS for complementary and noncomplementary case IDs represented statistically significant DFS distinctions for case IDs with CSs derived from both the extended and abbreviated approaches (FIGS. 30A and 30B), where case IDs representing BCR CDR3-mutant TP53 complementarity correlated with higher DFS rates. Case IDs representing complementarity based on the abbreviated approach exhibited a statistically significant increase in the DFS rate when compared to all remaining BRCA case IDs (FIG. 30C); moreover, there was no significant distinction between noncomplementary case IDs versus all remaining case IDs (FIG. 31), again using the abbreviated approach. The complementary case IDs representing the extended approach showed a trend towards higher DFS rates in comparison to all remaining (p=0.078, FIG. 32). And, the noncomplementary case IDs for the extended approach had a DFS rate similar to all remaining case IDs (FIG. 33).

To determine whether there was a difference in gene expression between complementary and noncomplementary case ID tumors, the RNASeq values for three categories of genes were obtained using the extended approach groupings: (i) immune function genes, (ii) proliferation effector genes, and (iii) apoptosis effector genes. Analyses revealed a statistically significant difference in RNASeq values for nine genes (FIG. 30D), all of which were related to, or highly specific for B-cells. To determine whether the difference in gene expression levels can be due to increased B-cell activation versus the presence of an increased number of cells, RNASeq values were normalized to BCR recombination read recoveries, which have been shown to be proportional to lymphocyte infiltrates. The results indicated no significant difference between complementary and noncomplementary case LDs with regard to RNASeq values per BCR recombination read recovered, indicating that the increased level of B-cell specific RNASeq values were due to an increased number of B-cells present in the CDR3-mutant TP53 complementary cases.

CSs for CDR3-mutant TP53 combinations were re-calculated using the extended and abbreviated approach and using CDR3 AA sequences represented by IGH recombination reads obtained from TCGA-BRCA RNASeq files. RNASeq-based, IGH CDR3s that were identical to WXS file recovered IGH CDR3s were eliminated from further consideration and analyses to maintain the highest standard for establishing a replicative value in assessing the RNASeq-based IGH CDR3s. Next, the proportion of case IDs representing complementary RNASeq-based, CDR3-mutant TP53 CSs was determined for the set of case IDs representing complementary CSs when the scores were obtained with WXS-based CDR3s. For example, for the extended CS calculation approach, out of 42 RNASeq-based CSs that were complementary, 38 (90%) represented case IDs that also had a complementary CS as determined by WXS-based CDR3s. Overall, this approach indicated a correspondence of the RNASeq-based CDR3s and the WXS-based CDR3s for the complementarity scoring process.

An assessment of a variety of clinical parameters potentially overlapping CDR3-mutant TP53 complementarity only indicated an inverse correlation with fraction genome altered (p=0.01), indicating that the complementary CSs were not a surrogate for increased mutation burdens.

To determine whether BRCA BCR CDR3s from complementary BCR CDR3-mutant TP53 combinations can be relevant in ovarian cancer, whether the recovery of BCR recombination reads reflected increased survival rates for the TCGA-OV dataset was determined (FIG. 30E). Also, KM curves based on aromaticity of OV cancer BCR CDR3 AA sequences showed that case IDs with IGH and IGL CDR3s with higher aromaticity had better DFS (FIG. 30F), indicating a chemical, and thereby antigen-interaction relevance to the BCRs at issue in ovarian cancer. Whether the OV and BRCA datasets represented common TP53 mutants was examined next. Comparison of TP53 mutants in the OV and BRCA datasets revealed that approximately 20% of OV TP53 mutants were the same as the TP53 mutants in the BRCA BCR CDR3-mutant TP53, complementary set, using the extended approach, indicating the more abundant and more readily detectable, BRCA-related, BCR CDR3s can be of relevance in the study and eventually treatment of ovarian cancer.

Overall, multiple approaches show that tumor resident, BCR CDR3-mutant TP53 complementarity is associated with better survival. Thus, the described electrostatic complementarity results in an increased immune response that is mediated by B-cells resulting in the increase in DFS rate. That immune response is associated with an increase in number of infiltrating B-cells, which in turn indicates that the increased number of B-cells is due to stimulation of cell division mediated by BCR activation by TP53 mutants.

### Abbreviations

AA, amino acid; BCR, B-cell receptor; BRCA, breast cancer; DFS, disease free survival; FGA, Fraction genome altered; GDC, genomic data commons; IG, immunoglobulin; KM, Kaplan-Meier; NCPR, net charge per residue; OV, ovarian cancer; TCGA, the cancer genome atlas; TILS, tumor infiltrating lymphocytes; WXS, whole exome sequence.

### Methods.

Obtaining IGH, IGK, and IGL CDR3 recombination reads from the cancer genome atlas (TCGA) WXS files. Mining of tumor exome (WXS) files for immune receptor V(D)J recombination reads has been extensively described and extensively benchmarked, particularly in the case of breast cancer (BRCA). Briefly, WXS files are pre-screened for BCR recombination

reads with a series of short nucleotide matches for both V and J regions close to the potential recombination junction, i.e., short sequences near the V gene segment 3′ end and near the J gene segment 5′ end. Then, the results of the pre-screen undergo a high stringency verification of V and J gene segments within a single read, based on a database available from imgt.org.

BRCA TP53 mutants. The TCGA-BRCA Mutect somatic mutation file, as well as the above indicated BRCA WXS files, were obtained from the genomic data commons (GDC) via database of genotypes and phenotypes (dbGaP) approved project number 6300.

NCPR-based complementarity scoring of BCR CDR3-mutant TP53 combinations via the extended approach. The complementarity scoring process is built on an approach developed in reference. Five types of TP53 mutations were considered: missense, nonsense, frameshift-insertions, frame-shift deletions and in-frame deletions. Missense mutations and in-frame deletions were calculated in the same manner, by determining the difference in charges, i.e., by subtracting the wild-type AA charge from the mutant AA charge. For nonsense mutations, frameshift-insertions, and frameshift-deletions, the sum of the AA sequence charge was calculated using all of AA upstream of the mutation site. The sum of the wild-type TP53 AA sequence was then subtracted from mutant sum. In rare cases, a case ID represented more than one TP53 mutant. In those cases, the average mutant minus wild-type difference was used for subsequent complementarity score (CS) calculations. To calculate the BCR CDR3-mutant TP53 CS for each case ID, the following procedure was used. First, the maximum and minimum NCPR values were obtained for each collection of BCR CDR3s recovered for each case ID. Next, for each CDR3-mutant TP53 case ID combination, the maximum and minimum CDR3 NCPR value was multiplied by the value generated for the TP53 mutant (mutant minus wild-type). The product minimum for each BCR CDR3-TP53 mutant case ID combination was then used as a final case ID CS. Thus, a negative product was representative of a complementary CDR3-mutant TP53 interaction and a positive product was representative of noncomplementary CDR3-TP53 mutant interaction.

NCPR-based complementarity scoring of BCR CDR3-mutant TP53 combinations via the abbreviated approach. CSs for BCR CDR3-mutant TP53 combinations using the abbreviated approach were generated in a similar manner to the extended approach. The difference between the approaches was in the valuation of nonsense, frameshift-deletions, frameshift-insertions and in-frame deletions. For the abbreviated approach, these mutations were assigned a value of zero. Then, the wild-type value of the AA was subtracted from zero. The remainder of the calculation of the CS was the same as for the extended approach. In sum, there were a total of 137 case IDs with either IGH, IGK or IGL recombination reads obtainable from BRCA tumor specimen WXS files and representing TP53 mutants. The extended approach yielded 42 case IDs representing CDR3-mutant TP53 mutant complementarity scores (CSs); the abbreviated approach yielded 36 such case IDs.

Kaplan-Meier (KM) analyses and comparison of gene expression levels using RNASeq values. The KM analyses were conducted using GraphPad Prism, and the TCGA-BRCA clinical data were obtained from cbioportal.org. The RNAseq data representing BRCA tumor samples were also obtained from cbioportal.org.

N-proportion analyses. The N-proportion analyses were conducted using the webtool at www.medcalc.net/statisticaltests/comparison_of_proportions.php.

Code availability. All novel computer code used in this report has been deposited at github.com/bchobrut-USF/lgg_idh1.

### Example 7. Electrostatic Complementarity Between T-Cell Receptors and MACF1 Mutants Represents a Survival Advantage in Patients with Muscle Invasive Bladder Cancer

Mutant amino acids in tumor cells are presumed to elicit an anti-tumor immune response, primarily mediated by T-cells. Thus, the amino acid sequences for the T-cell receptors were obtained in muscle invasive bladder cancer (MIBC) patients to determine whether computational approaches can chemically link the T-cell receptors (TCR) to such mutant amino acids. This approach was applied to MIBC patients with mutations in the MACF1 gene, a microtubule-actin cross-linking factor and positive regulator of the Wnt signaling pathway, to determine if TCR-mutant amino acid chemical linkage correlates with clinical features and survival outcomes.

### Methods

The amino acid sequences of the TCR-antigen binding site from T-cells of MIBC patients were obtained from The Cancer Genome Atlas (TCGA). The electrostatic charge of these amino acids sequences, termed the complementary determining region-3 (CDR3) was assessed. The net change in electrostatic charge caused by the mutant amino acids was obtained in the tumor cells of the matching patients. To determine whether the CDR3 electrostatic charges were complementary to the corresponding amino acids charges, an original Python program was created for outputting the charge relationships and their connection to survival rates. Overall and disease free survival were analyzed using Kaplan-Meier plots and a log rank-test was applied to assess for survival differences. Variations in clinical characteristics were examined using Chi-squared analysis.

### Results

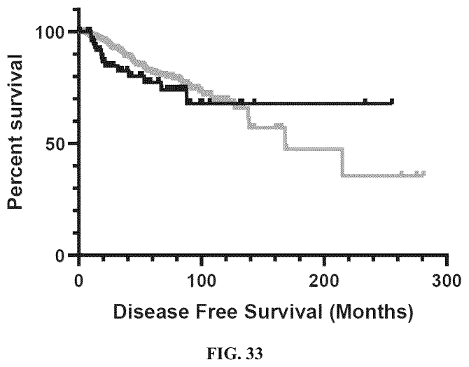

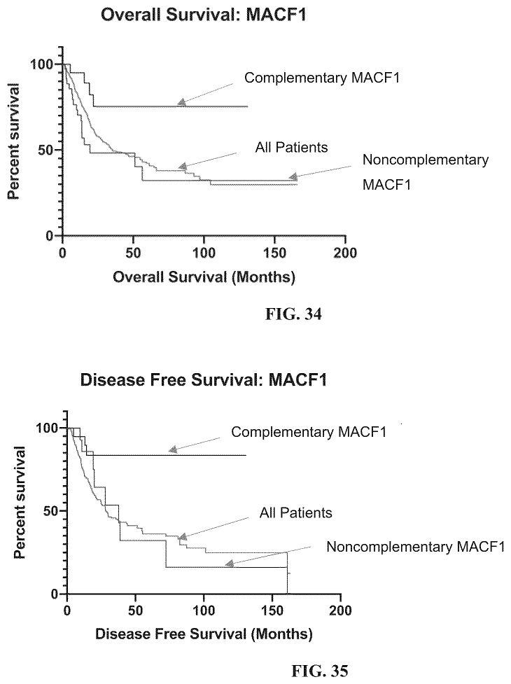

Of the 413 patients with MIBC in the TCGA, 53 had mutations in the MACF1 gene. Electrostatic complementarity for the MACF1 mutant amino acids and the corresponding TCR CDR3s (from the same patients) was found in 23 of these patients. The patients with electrostatic complementarity had prolonged overall and disease free survival when compared with patients with non-complementary TCR CDR3-MACF1 mutants (p=0.007 and 0.016, respectively; FIGS. 34 and 35). Overall and disease free survival for patients with non-complementary TCR CDR3-MACF1 mutants were similar to that of all MIBC patients in the TCGA (p=0.233 and 0.888), whereas patients with complementarity had significantly improved survivals compared to all patients (p=0.013 and 0.003). Patients with TCR-MACF1 complementarity were more likely to be male and white compared to all other patients in TCGA dataset (p=0.049 and 0.025 respectively; Table 12). Of note, those with complementarity were more likely than non-complementary TCR CDR3-MACF1 mutants to have incidental prostate cancer (p=0.001).

Conclusion: Electrostatic complementarity between TCR CDR3 and MACF1 mutants is associated with improved survival odds in patients with MIBC.

TABLE 12
Complementary All Other

| | Complementary | | All Other | | | |
|---|---|---|---|---|---|---|
| Characteristic | MACF1 N (%) | | Patients N (%) | | χ² | P-Value |
| Race | | | | | | |
| White | 23 | (100) | 303 | (81.89) | 5.023 | 0.025 |
| Non-White | 0 | (0) | 67 | (18.11) | | |
| Stage* | | | | | | |
| I | 0 | (0) | 2 | (0.52) | 1.808 | 0.613 |
| II | 5 | (21.74) | 128 | (32.99) | | |
| III | 8 | (34.78) | 133 | (34.28) | | |
| IV | 10 | (43.48) | 125 | (32.23) | | |
| Gender | | | | | | |
| Male | 21 | (91.30) | 282 | (72.68) | 3.888 | 0.049 |
| Female | 2 | (8.70) | 106 | (27.32) | | |
| Smoking | | | | | | |
| History | | | | | | |
| Yes | 16 | (80) | 271 | (71.69) | 0.652 | 0.419 |
| No | 4 | (20) | 107 | (28.31) | | |

| | | | | |
|---|---|---|---|---|
| Incidental | | | | |
| Prostate | | | | |
| Cancer | | | | |
| Yes | 42.105 | 5.714 | 3.01 | 0.0828 |
| No | 57.895 | 94.286 | | |

*Staging based on American Joint Committee on Cancer

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
SEQ ID NO: 1
CITVLSSDSGGSNYKLTF,
SEQ ID NO: 2
CVVTPGGGNNRKLIW,
SEQ ID NO: 3
CASSLYPNTVELFF,
SEQ ID NO: 4
CASSIGQWRRPQHF,

1. A method for detecting a survival rate of a subject having a cancer, comprising:
isolating a nucleic acid from a biological sample derived from the subject;
sequencing a first polynucleotide encoding a complementarity determining region (CDR) domain of an antigen-binding protein and a second polynucleotide encoding a protein associated with the cancer,
detecting and grouping together two or more common chemical features from the CDR, wherein the two or more chemical features are selected from an isoelectric point, a fraction of positive amino acid residues, or a net charge per residue (NCPR);
detecting a complementarity score of the CDR domain and the protein associated with the cancer, comprising multiplying the NCPR of the CDR domain by a value of change in charge due to an amino acid substitution in the protein associated with the cancer and further by “−1”; and
detecting the survival rate when the two or more chemical features are reduced and the complementary score is increased relative to a reference control.

2. The method of claim 1, wherein the antigen-binding protein comprises a T cell receptor (TCR) alpha chain or a TCR beta chain.

3. (canceled)

4. (canceled)

5. The method of claim 1, wherein the CDR domain is a CDR3 domain.

6. The method of claim 1, wherein the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

7. The method of claim 1, wherein the protein associated with the cancer is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

8. The method of claim 1, further comprising administering to the subject an anti-cancer agent.

9. The method of claim 8, wherein the anti-cancer agent is selected from the group consisting of cordycepin, fenretinide, imiquimod, dabrafenib, encorafenib, anthracyclines, taxanes, ixabepilone, eribulin, fulvestrant, exemestane, pertuzumab, ado-trastuzumab emtansine, lapatinib, neratinib, everolimus, olaparib, talazoparib, alpelisib, atezolizumab, albumin-bound paclitaxel, pemetrexed, bevacizumab, ramucirumab, mitomycin, durvalumab, avelumab, erdafitinib, epirubicin, temozolomide, trabectedin, and pazopanib.

10.-18. (canceled)

19. A method for treating a cancer in a subject, comprising:
isolating a nucleic acid from a biological sample derived from the subject;
sequencing a first polynucleotide encoding a complementarity determining region (CDR) domain of an antigen-binding protein and second polynucleotide encoding a protein associated with the cancer;
detecting and grouping together two or more common chemical features from the CDR, wherein the two or more chemical features are selected from an isoelectric point, a fraction of positive amino acid residues, or a net charge per residue (NCPR);
detecting a complementarity score of the CDR domain and the protein associated with the cancer, comprising multiplying the NCPR of the CDR domain by a value of change in charge due to an amino acid substitution in the protein associated with the cancer and further by “−1”; and
administering to the subject a therapeutically effective amount of an anti-cancer agent when the two or more chemical features are increased and the complementary score is decreased relative to a reference control.

20. The method of claim 20, wherein the antigen-binding protein comprises a T cell receptor (TCR) alpha chain or a TCR beta chain.

21. (canceled)

22. (canceled)

23. The method of claim 20, wherein the CDR domain is a CDR3 domain.

24. The method of claim 20, wherein the cancer is selected from the group consisting of low-grade glioma, stomach adenocarcinoma, esophageal cancer, melanoma, lung squamous cell carcinoma, lung adenocarcinoma, breast cancer, cervical squamous cell carcinoma, bladder cancer, muscle invasive bladder cancer, and soft tissue sarcoma.

25. The method of claim 20, wherein the protein associated with the cancer is selected from the group consisting of isocitrate dehydrogenase 1 (IDH1), Phosphatidylinositol-4,5-Bisphosphate 3-Kinase Catalytic Subunit Alpha (PIK3CA), B-Raf proto-oncogene (BRAF), Dynein heavy chain 9 (DNAH9), myosin heavy chain 1 (MYH1), Tenascin-R (TNR), Teneurin-1 (TNM1), Plexin-A4 (PLXNA4A), Microtubule-actin cross-linking factor 1 (MACF1), Tumor protein p53 (TP53), ATP-dependent helicase ATRX (ATRX), Neuroblastoma RAS viral oncogene homolog (NRAS), and Retinoblastoma protein (RB1).

26. (canceled)

27. The method of claim 19, wherein the anti-cancer agent is selected from the group consisting of cordycepin, fenretinide, imiquimod, dabrafenib, encorafenib, anthracyclines, taxanes, ixabepilone, eribulin, fulvestrant, exemestane, pertuzumab, ado-trastuzumab emtansine, lapatinib, neratinib, everolimus, olaparib, talazoparib, alpelisib, atezolizumab, albumin-bound paclitaxel, pemetrexed, bevacizumab, ramucirumab, mitomycin, durvalumab, avelumab, erdafitinib, epirubicin, temozolomide, trabectedin, and pazopanib.

28.-37. (canceled)
